(19) United States
(12) Patent Application Publication
(10) Pub. No.: US 2022/0152276 A1
(43) Pub. Date: May 19, 2022

(54) Improved Decellularization of Isolated Organs

(71) Applicant: Miromatrix Medical Inc., Eden Prairie, MN (US)

(72) Inventors: Anne YOUNG, Minneapolis, MN (US); Aleksandr KATANE, Chaska, MN (US); Ben STEINER, Bloomington, MN (US); Brian PERRAULT, Chaska, MN (US); Dominique Seetapun DAVIDOW, Minnetonka, MN (US); Jeffrey ROSS, Chaska, MN (US)

(21) Appl. No.: 17/440,914

(22) PCT Filed: Mar. 20, 2020
(86) PCT No.: PCT/US2020/024052

§ 371 Date: Sep. 20, 2021

Related U.S. Application Data
(60) Provisional application No. 62/854,104, filed on May 29, 2019.
(60) Provisional application No. 62/821,620, filed on Mar. 21, 2019.

(51) Int. Cl. A61L 27/36; A61L 27/38

(52) U.S. Cl. CPC A61L 27/3687; A61L 27/3834; A61L 27/3625; A61L 27/3808

(57) ABSTRACT

Disclosed herein are compositions and methods to decellularize an isolated organ or portion thereof. Also disclosed herein are compositions and methods for treatment of disease utilizing a decellularized or recellularized organ. Also disclosed herein are methods of improving decellularization and/or recellularization of an isolated organ or portion thereof.

### CROSS REFERENCE

This application claims the benefit of U.S. Provisional Application No. 62/821,620, filed Mar. 21, 2019, which is incorporated by reference herein in its entirety.

### SUMMARY

Disclosed herein are methods, which can comprise contacting an isolated organ or portion thereof with a liquid. In some embodiments, (a) a liquid can be at least partially degassed; (b) a liquid can comprise a detergent and/or a radical-generating compound; (c) an isolated organ or portion thereof can be from a mammal that was fed within about ten hours prior to removal of an isolated organ or portion thereof; or (d) any combination of (a)-(c). In some embodiments, a method can comprise at least two of: (a), (b) or (c). In some embodiments, a method can comprise (a), (b), and (c). In some embodiments, an isolated organ or portion thereof can be from a mammal. In some embodiments, a mammal can be a non-human mammal. In some embodiments, a non-human mammal can be a pig, a sheep, a goat, a cow, a dog, a cat, or a monkey. In some embodiments, a mammal can be a human mammal. In some embodiments, an isolated organ or portion thereof can be at least part of a liver, a lung, a heart, a kidney, a bladder, a pancreas, a spleen, a uterus, a portion of any of these, or any combination thereof. In some embodiments, a contacting can comprise (i) perfusing a liquid in at least a portion of an isolated organ or portion thereof, (ii) injecting a liquid in at least a portion of an isolated organ or portion thereof, (iii) spraying a liquid on at least a portion of an isolated organ or portion thereof, (iv) submerging an isolated organ or portion thereof in a liquid, or (v) any combination thereof. In some embodiments, an isolated organ or portion thereof can be cannulated prior to, during, or after a contacting. In some embodiments, a liquid can comprise an at least partially degassed decellularization media. In some embodiments, contacting with an at least partially degassed decellularized media can form an at least partially decellularized isolated organ or portion thereof. In some embodiments, an at least partially decellularized isolated organ or portion thereof can comprise an extracellular matrix. In some embodiments, an extracellular matrix can comprise a vasculature bed. In some embodiments, following a contacting, a vasculature bed can remain at least partially intact. In some embodiments, a liquid can be at least partially degassed. In some embodiments, an extracellular matrix can have an increased compressive modulus relative to an otherwise comparable extracellular matrix produced by contact with a non-degassed liquid for a comparable time period. In some embodiments, a method can comprise at least (a). In some embodiments, an isolated organ or portion thereof can be contacted by an at least partially degassed liquid for at least about two hours. In some embodiments, a dissolved gas in an at least partially degassed liquid can have a concentration of less than about 1 milligram (mg) per liter (L). In some embodiments, a dissolved gas can be selected from the group consisting of: oxygen, nitrogen, carbon monoxide, carbon dioxide, a noble gas, and any combination thereof. In some embodiments, an isolated organ or portion thereof after a contacting can comprise fewer air emboli, fewer microbubbles, less pigmentation, or any combination thereof relative to an otherwise comparable isolated organ or portion thereof produced by contacting for a comparable amount of time with an otherwise comparable liquid that has not been at least partially degassed. In some embodiments, an at least partially degassed liquid can comprise an at least partially degassed decellularization media, thereby forming an at least partially decellularized isolated organ or portion thereof. In some embodiments, an at least partially decellularized isolated organ or portion thereof after a contacting with an at least partially degassed decellularization media can contain fewer cells than an otherwise comparable isolated organ or portion thereof decellularized with an otherwise comparable non-degassed decellularization media. In some embodiments, an amount of time sufficient to produce an at least partially decellularized isolated organ or portion thereof with an at least partially degassed decellularization media can be less than an amount of time to produce a substantially decellularized isolated organ or portion thereof with a non-degassed decellularization media. In some embodiments, a method can comprise at least (b). In some embodiments, a radical generating compound can be of formula R¹—O—O—R², wherein R¹ and R² can independently be H, C₁-C₆ alkyl, substituted C₁-C₆ alkyl, aryl, substituted aryl, benzyl, substituted benzyl, C(═O)A¹, C(═O)OA², wherein A¹ and A² can independently be H, C₁-C₆ alkyl or substituted C₁-C₆ alkyl. In some embodiments, R¹ can be H and R² can be C(═O)CH₃. In some embodiments, a radical generating compound can be a peracid. In some embodiments, a peracid can comprise: peroxyacetic acid, peroxyoctanoic acid, a sulfoperoxycarboxylic acid, peroxysulfonated oleic acid, peroxyformic acid, peroxyoxalic acid, peroxypropanoic acid, peroxybutanoic acid, peroxypentanoic acid, peroxyhexanoic acid, peroxyadipic acid, peracetic acid, perlactic acid, peroxycitric, peroxybenzoic acid, or any combination thereof. In some embodiments, a peracid can comprise peracetic acid. In some embodiments, a peracid can be present in an amount that comprises at least about 10 ppm, 25 ppm, 50 ppm, 75 ppm, 90 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 900 ppm, 1000 ppm, 1200 ppm, 1400 ppm, 1500 ppm, 1700 ppm, 2000 ppm, 2200 ppm, 2500 ppm, 2750 ppm, 3000 ppm, 3200 ppm, 3500 ppm, 3750 ppm, or 4000 ppm. In some embodiments, a contacting can produce an at least partially decellularized isolated organ or portion thereof. In some embodiments, an at least partially decellularized isolated organ or portion thereof can comprise a reduced amount of cells as compared to an otherwise comparable decellularized isolated organ or portion thereof contacted with an otherwise comparable liquid that does not comprise a peracid for a comparable time period. In some embodiments, a reduced amount of cells can comprise a reduction of at least about 85%, at least about 90%, at least about 92%, at least about 95%, at least about 97%, at least about 98%, at least about 99%, or at least about 99.5% of cells from the isolated organ or portion thereof. In some embodiments, a contacting can comprise perfusing. In some embodiments, a flow rate of perfusion can be increased compared to a flow rate of perfusion of an otherwise comparable liquid that does not comprise peracid. In some embodiments, a flow rate can be increased without substantially compromising an integrity of an extracellular matrix of an isolated organ or portion thereof. In some embodiments, a method can comprise at least (c). In some embodiments, an isolated organ or portion thereof can comprise a reduced particulate level as compared to an otherwise comparable isolated organ or portion thereof produced from an animal which had not been fed within about 12 hours, about 10 hours, about 8 hours, about 6 hours, about 4 hours, about 2 hours, or about 1 hour prior to removal of the isolated organ or portion thereof from the animal. In some embodiments, a reduced particulate level can be reduced by at least about: 90%, 85%, 80%, 75%, 70%, 65%, 60%, 55%, 50%, 45%, 40%, 35%, 30%, 25%, 20%, 15%, 10%, 5%, 1% as compared to an otherwise comparable isolated organ or portion thereof produced from an animal which had not been fed within about 12 hours, about 10 hours, about 8 hours, about 6 hours, about 4 hours, about 2 hours, or about 1 hour prior to removal of the isolated organ or portion thereof from the animal. In some embodiments, a reduced particulate level can be determined by visual examination, determining an outflow, determining turbidity, microscopy analysis, or any combination thereof. In some embodiments, a method can further comprise washing an isolated organ or portion thereof with a wash media prior to a contacting. In some embodiments, a wash media can comprise a hypertonic solution. In some embodiments, a hypertonic solution can comprise saline. In some embodiments, a wash media can comprise a disinfecting solution. In some embodiments, a method can further comprise contacting an isolated organ or portion thereof with a second liquid. In some embodiments, a contacting with a second liquid can occur after a contacting with a liquid. In some embodiments, a second liquid can be an at least partially degassed liquid. In some embodiments, a second liquid can comprise a wash media. In some embodiments, an isolated organ or portion thereof can contain less than about 1% weight per weight of a detergent after contacting of an isolated organ or portion thereof with a second liquid. In some embodiments, after a contacting with a second liquid, blood can flow through a greater volume of vasculature of an isolated organ or portion thereof as compared to an otherwise comparable isolated organ or portion thereof produced using a non-degassed second liquid. In some embodiments, a method can further comprise contacting the at least partially decellularized isolated organ or portion thereof with a regeneration media. In some embodiments, a regeneration media can be an at least partially degassed cellular regeneration media. In some embodiments, a contacting with an at least partially degassed cellular regeneration media can form an at least partially recellularized organ or portion thereof. In some embodiments, an at least partially degassed cellular regeneration media can comprise a population of regenerative cells. In some embodiments, a contacting with an at least partially degassed cellular regeneration media can result in a higher degree of recellularization relative to an otherwise comparable isolated organ or portion thereof produced using a non-degassed cellular regeneration media.

Also disclosed herein in some embodiments, are at least partially decellularized isolated organs or portions thereof generated by a method as described herein

Also disclosed herein in some embodiments, are at least partially recellularized mammalian isolated organs or portions thereof generated by a method as described herein.

Also disclosed herein in some embodiments, are systems. A system described herein can comprise: (a) an at least partially decellularized isolated organ or portion generated by a method described herein, (b) a device for moving fluid, (c) a cannula, or (d) a port. In some embodiments, a system can comprise any combination of (a), (b), (c), and (d), including at least two, three, or all of (a), (b), (c), and (d). In some embodiments, a system can further comprise a degassed decellularization media. In some embodiments, a system can further comprise a degassed wash media.

Also disclosed herein in some embodiments, are kits. A kit described herein can comprise an at least partially decellularized isolated organ or portion thereof generated by a method described herein. In some embodiments, a kit can further comprise, (a) at least one cannula, (b) a liquid, (c) instructions for recellularizing an at least partially decellularized isolated organ or portion thereof, (d) instructions for introducing an at least partially recellularized isolated organ or portion thereof in a subject, or (e) any combination thereof. In some embodiments, an at least partially decellularized isolated organ or portion thereof can be in a packaging.

### INCORPORATION BY REFERENCE

All publications, patents, and patent applications mentioned in this specification are herein incorporated by reference to the same extent as if each individual publication, patent, or patent application was specifically and individually indicated to be incorporated by reference. U.S. Pat. Nos. 10,213,525 and 10,220,056 are incorporated by reference herein in their entirety.

### BRIEF DESCRIPTION OF THE DRAWINGS

The novel features of exemplary embodiments are set forth with particularity in the appended claims. A better understanding of the features and advantages will be obtained by reference to the following detailed description that sets forth illustrative embodiments, in which exemplary embodiments are utilized, and the accompanying drawings of which:

FIG. 1 shows a schematic showing an initial preparation for a decellularization of a heart. An aorta, pulmonary artery, and superior vena cava are cannulated (A, B, C, respectively), and an inferior vena cava, brachiocephalic artery, left common carotid artery, and left subclavian artery are ligated. Arrows indicate the direction of perfusion in antegrade and retrograde.

FIG. 2 shows decellularization of an adult porcine heart over an about 48 hour time period. A 6-month-old porcine heart undergoing perfusion decellularization over a period of 48 hours. At least some native structure and at least some vasculature can be preserved after decellularization. After 48 hours, a heart can be at least partially decellularized.

FIG. 3 shows decellularization of an adult porcine liver over 24 hours. A 6-month-old porcine liver undergoing perfusion decellularization over a period of 24 hours. A native structure and vasculature remain preserved after decellularization.

FIG. 4A shows a porcine liver after decellularization having a particulate percent from 0-5%. FIG. 4B shows a porcine liver after decellularization having a particulate percent of 20%. FIG. 4C shows a porcine liver after decellularization having a particulate percent from 40-50%. FIG. 4D shows a porcine liver after decellularization having a particulate percent from 50-60%. FIG. 4E shows a porcine liver after decellularization having a particulate percent of 60%. FIG. 4F shows a porcine liver after decellularization having a particulate percent of 70%. FIG. 4G shows a porcine liver after decellularization having a particulate percent of 80%. FIG. 4H shows a porcine liver after decellularization having a particulate percent from 95-100%.

FIG. 5 shows an elution of particulate into a tubing connected to a portal vein cannula of a liver with visual particulate undergoing perfusion decellularization when perfusion can be stopped for 10 minutes after about 24 hours of perfusion decellularization.

FIG. 6A shows decellularization of an adult porcine lung over 48 hours. A 6-month-old porcine lung undergoing perfusion decellularization over a period of 48 hours. A native structure and vasculature can be preserved after decellularization. FIG. 6B depicts decellularization after 48 hours, a lung can be completely decellularized.

FIG. 7A shows decellularization of an adult porcine kidney over 48 hours. A 6-month-old porcine kidney undergoing perfusion decellularization over a period of 48 hours. A native structure and vasculature can be preserved after decellularization. FIG. 7B depicts decellularization of a kidney after 48 hours, a kidney can be completely decellularized.

FIG. 8A shows an absorbance profile of a solution surrounding a liver with visible particulate (L281) after a 30 minute re-circulating water rinse. FIG. 8B shows an absorbance profile of effluent in a solution surrounding a liver with visible particulate L274 after 15 minutes of a re-circulating water wash.

FIG. 9 depicts processing of native and decellularized kidney scaffolds using DNA-binding florescence staining. Examination was conducted under immunofluorescent microscopy.

FIGS. 10A-C show an increase in the flow rate after a radical generating compound (e.g., peracetic acid) can be introduced into a perfusion decellularization process. An increase in flow rate can occur after addition of a radical generating compound during perfusion decellularization of a porcine liver at a set pressure of 12 mmHg (FIGS. 10A-C). A decellularization apparatus allows for a defined pressure to be specified and a flow rate can be adjusted in order to maintain a defined pressure.

FIG. 11 shows a plot of the normalized peak load force values for samples that can be stretched in increments of 4% strain and allowed to relax.

FIG. 12A depicts a representation of endothelial seeding used in the histological analysis.

FIG. 12B depicts representative images of the bioreactor, histology after 21 days, and an exemplary vessel surface.

FIG. 13 depicts immunofluorescent staining, which demonstrates the distribution and engraftment of native kidney cells throughout the kidney in the proper location.

FIG. 14 depicts improvements in flow rates of perfusion at constant pressure with the addition of a radical generating compound PAA during a decellularization process workflow.

### DETAILED DESCRIPTION

Overview

In some embodiments disclosed herein, are methods to substantially decellularize isolated organs, or portions thereof. In some embodiments, a decellularization method can comprise perfusing an isolated organ or portion thereof with a degassed cellular disruption media under a pressure of less than about 700 torr from about 15° C. to about 95° C. prior to perfusing, thereby substantially removing dissolved gas from a degassed cellular disruption media. In some embodiments, a degassed cellular disruption media can comprise a detergent. In some embodiments, a detergent can comprise sodium dodecyl sulfate, or polyethylene glycol, or Triton-X 100. In some embodiments, an amount of microbubbles can be reduced during a perfusion as compared to an otherwise comparable cellular disruption media with more than about 8 parts per million (ppm) of oxygen when stored at a temperature of about 25° C. In some embodiments, a degassed cellular disruption media can be connected to a vacuum pump for about 10 minutes prior to perfusing. In some embodiments, a degassed cellular disruption media can be placed at a temperature of from about 4° C. to about 95° C. prior to perfusing. In some embodiments, a dissolved gas can comprise oxygen or nitrogen. In some embodiments, an amount of time sufficient to produce an at least partially decellularized isolated organ or portion thereof with a degassed cellular disruption media can be less than an amount of time to produce an at least partially decellularized isolated organ or portion thereof with a non-degassed cellular disruption media. In some embodiments, an amount by volume of a degassed cellular disruption media that can be sufficient to produce an at least partially decellularized isolated organ or portion thereof can be less than an amount by volume of non-degassed cellular disruption media required to produce an at least partially decellularized isolated organ or portion thereof. In some embodiments, an amount by weight of degassed cellular disruption media that can be sufficient to produce an at least partially decellularized isolated organ or portion thereof can be less than an amount by weight of non-degassed cellular disruption media required to produce an at least partially decellularized isolated organ or portion thereof. In some embodiments, an at least partially decellularized isolated organ or portion thereof can comprise an extracellular matrix. In some embodiments, an extracellular matrix can comprise fibronectin, fibrillin, laminin, elastin, a collagen family protein, a glycosaminoglycan, a ground substance, a reticular fiber or thrombospondin. In some embodiments, a collagen family protein can comprise collagen I, collagen II, collagen III, or collagen IV. In some embodiments, an at least partially decellularized isolated organ or portion thereof can comprise a vasculature bed. In some embodiments, an isolated organ or portion thereof can be from a mammal. In some embodiments, a mammal can be a rodent, a pig, a rabbit, cattle, a sheep, a dog, a cat, or a human. In some embodiments, an isolated organ or portion thereof can be a heart, liver, lung, brain, pancreas, spleen, kidney, uterus, or bladder. In some embodiments, perfusing can be antegrade. In some embodiments, perfusing can be retrograde. In some embodiments, degassed cellular disruption media can be recirculated during perfusion.

Also disclosed herein is an isolated organ or portion thereof which can comprise an extracellular matrix, which can be cannulated at one or more cavities, vessels, and/or ducts. In some embodiments, a degassed cellular disruption media can be perfused to yield an at least partially decellularized cannulated isolated organ or portion. In some embodiments, an at least partially decellularized cannulated organ or portion thereof can comprise an at least partially decellularized extracellular matrix, which can comprise an exterior surface and a vascular tree. In some embodiments, a degassed cellular disruption media can comprise less than about 8 parts per million (ppm) of oxygen when stored at a temperature of about 25° C. In some embodiments, a degassed cellular disruption media can allow for increased removal of cells in a shorter time as compared to an otherwise comparable cellular disruption media with more than about 8 ppm of oxygen when stored at a temperature of about 25° C. In some embodiments, a degassed cellular disruption media can comprise a detergent. In some embodiments, a detergent can comprise sodium dodecyl sulfate, or polyethylene glycol, or Triton-X 100. In some embodiments, an amount of microbubbles can be reduced during perfusion as compared to an otherwise comparable cellular disruption media with more than about 8 parts per million (ppm) of oxygen when stored at a temperature of about 25° C. In some embodiments, a degassed cellular disruption media can be connected to a vacuum pump for about 10 minutes prior to perfusing. In some embodiments, a degassed cellular disruption media can be placed at a temperature of from about 4° C. to about 95° C. prior to perfusing. In some embodiments, a dissolved gas can comprise oxygen or nitrogen. In some embodiments, an amount of time sufficient to produce an at least partially decellularized isolated organ or portion thereof with a degassed cellular disruption media can be less than an amount of time to produce an at least partially decellularized isolated organ or portion thereof with a non-degassed cellular disruption media. In some embodiments, an amount by volume of a degassed cellular disruption media that can be sufficient to produce an at least partially decellularized isolated organ or portion thereof can be less than an amount by volume of non-degassed cellular disruption media required to produce an at least partially decellularized isolated organ or portion thereof. In some embodiments, an amount by weight of a degassed cellular disruption media that can be sufficient to produce an at least partially decellularized isolated organ or portion thereof can be less than an amount by weight of non-degassed cellular disruption media required to produce an at least partially decellularized isolated organ or portion thereof. In some embodiments, an at least partially decellularized isolated organ or portion thereof can comprise an extracellular matrix. In some embodiments, an extracellular matrix can comprise fibronectin, fibrillin, laminin, elastin, a collagen family protein, a glycosaminoglycan, a ground substance, a reticular fiber, or thrombospondin. In some embodiments, a collagen family protein can be collagen I, collagen II, collagen III, or collagen IV. In some embodiments, an at least partially decellularized isolated organ or portion thereof can comprise a vasculature bed. In some embodiments, an isolated organ or portion thereof can be from a mammal. In some embodiments, a mammal can be a rodent, a pig, a rabbit, cattle, a sheep, a dog, a cat, or a human. In some embodiments, an isolated organ or portion thereof can be a heart, liver, lung, brain, pancreas, spleen, kidney, uterus, or bladder. In some embodiments, perfusing can be antegrade. In some embodiments, perfusing can be retrograde. In some embodiments, degassed cellular disruption media can be recirculated during perfusion.

Also disclosed herein is a composition that can comprise an at least partially decellularized mammalian isolated organ or portion thereof. In some embodiments, an isolated organ or portion thereof can have an at least partially decellularized extracellular matrix having an exterior surface and a vascular tree. In some embodiments, an at least partially decellularized extracellular matrix of a mammalian isolated organ or portion thereof can comprise a cellular disruption media with less than about 8 parts per million (ppm) of oxygen when stored at a temperature of about 25° C.

Also disclosed herein is a composition that can comprise an isolated decellularized mammalian isolated organ or portion thereof that has been perfused with a cellular disruption media. In some embodiments, a cellular disruption media can comprise a dissolved oxygen concentration of less than about 8 parts per million (ppm). In some embodiments, an isolated decellularized mammalian isolated organ or portion thereof can comprise at most about 10% of native cells after perfusing with a cellular disruption media for at most 10 hours. In some embodiments, a system can include an at least partially decellularized mammalian isolated organ or portion thereof. In some embodiments, a system can include an isolated decellularized mammalian isolated organ or portion thereof. In some embodiments, a system can comprise a peristaltic pump. In some embodiments, a system can comprise a kit which can comprise an isolated decellularized mammalian isolated organ or portion thereof, at least one cannula, reagents, instructions for recellularizing an isolated decellularized mammalian isolated organ or portion thereof, and/or instructions for introducing an isolated decellularized mammalian isolated organ or portion thereof in a subject. In some embodiments, a kit can be in a packaging. In some embodiments, packaging can be sterile.

Also disclosed herein is a method of providing an at least partially decellularized isolated organ or portion thereof, wherein an at least partially decellularized isolated organ or portion thereof can comprise a vasculature bed, and wherein an at least partially decellularized isolated organ or portion thereof can be substantially free of endothelial cells. In some cases, a method can include producing an at least partially recellularized organ or portion thereof, wherein producing can comprise perfusing an at least partially decellularized isolated organ or portion thereof with a degassed cellular regeneration media; wherein a degassed cellular regeneration media can comprise less than about 8 parts per million (ppm) of oxygen when stored at a temperature of about 25° C. In some embodiments, degassed cellular regeneration media can comprise a population of cells. In some embodiments, a population of cells can comprise totipotent cells, pluripotent cells, multipotent cells, or any combination thereof. In some embodiments, a population of regenerative cells can comprise undifferentiated cells, partially differentiated cells, fully differentiated cells, or a combination thereof. In some embodiments, a population of regenerative cells can be independently from a mammal. In some embodiments, a mammal can be a rodent, a pig, a rabbit, cattle, a sheep, a dog, or a human. In some embodiments, a population of regenerative cells can be embryonic stem cells, umbilical cord blood cells, tissue-derived stem or progenitor cells, bone marrow-derived stem or progenitor cells, blood-derived stem or progenitor cells, mesenchymal stem cells (MSC), skeletal muscle-derived cells, multipotent adult progenitor cells (MAPC), cardiac stem cells (CSC), multipotent adult cardiac-derived stem cells, cardiac fibroblasts, cardiac microvasculature endothelial cells, aortic endothelial cells, bone marrow mononuclear cells (BM-MNC), endothelial progenitor cells (EPC), or any combination thereof. In some embodiments, a population of regenerative cells can comprise at least about 1,000 regenerative cells. In some embodiments, an amount of degassed cellular regeneration media in perfusing can comprise at least about 1,000 regenerative cells per mg of substantially decellularized isolated organ or portion thereof. In some embodiments, a degassed cellular generation media can comprise a growth factor. In some embodiments, a growth factor can be VEGF, DKK-1, FGF, BMP-1, BMP-4, SDF-1, IGF, HGF, or any combination thereof. In some embodiments, a degassed cellular generation media can comprise an immune modulating agent. In some embodiments, an immune modulating agent can be a cytokine, a glucocorticoid, an IL2R antagonist, a leukotriene antagonist, or any combination thereof. In some embodiments, an at least partially decellularized isolated organ or portion thereof can further comprise an extracellular matrix. In some embodiments, an extracellular matrix can comprise fibronectin, fibrillin, laminin, elastin, a collagen family protein, a glycosaminoglycan, a ground substance, a reticular fiber or thrombospondin. In some embodiments, a collagen family protein can be collagen I, collagen II, collagen III, or collagen IV. In some embodiments, an at least partially decellularized isolated organ can be from a mammal. In some embodiments, a mammal can be a rodent, a pig, a rabbit, cattle, a sheep, a dog, a cat, or a human. In some embodiments, an at least partially recellularized organ or portion thereof can be a heart, liver, lung, brain, pancreas, spleen, kidney, uterus, or bladder. In some embodiments, perfusing can be antegrade. In some embodiments, perfusing can be retrograde.

Also disclosed herein is an at least partially recellularized mammalian isolated organ or portion thereof which can comprise a cellular regeneration media with less than about 8 parts per million (ppm) of oxygen when stored at a temperature of about 25° C.

Also disclosed herein in some embodiments, are compositions and methods of isolated whole organ decellularization as well as decellularization of isolated whole organ portions. Also disclosed herein in some embodiments, are improvements of decellularization utilizing methods and compositions. In some embodiments, compositions and methods of improved decellularization disclosed herein can result in higher efficiency decellularization and improved recellularization of compositions described herein.

### Definitions

Unless defined otherwise, all technical and scientific terms used herein have the same meaning as is commonly understood by one of ordinary skill in the art.

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof as used herein mean “comprising”.

The term “about” and its grammatical equivalents in relation to a reference numerical value and its grammatical equivalents as used herein can include a range of values plus or minus 10% from that value. For example, the amount “about 10” includes amounts from 9 to 11. The term “about” in relation to a reference numerical value can also include a range of values plus or minus: 10%, 9%, 8%, 7%, 6%, 5%, 4%, 3%, 2%, or 1% from that value.

The term “substantially” as used herein can refer to a value approaching 100% of a given value. In some embodiments, the term can refer to an amount that can be at least about 90%, 91%, 92%, 93%, 94%, 95%, 96%, 97%, 98%, 99%, 99.9%, or 99.99% of a total amount. In some embodiments, the term can refer to an amount that can be about 100% of a total amount.

The term “decellularized” or “decellularization” as used herein can refer to a biostructure (e.g., an isolated organ or portion thereof, or tissue), from which the cellular and tissue content has been removed leaving behind an intact acellular infra-structure. Organs such as a kidney can be composed of various specialized tissues. Specialized tissue structures of an organ, or parenchyma, can provide specific function associated with the organ. Supporting fibrous network of an isolated organ can be a stroma. Most organs can have a stromal framework composed of unspecialized connecting tissue which supports the specialized tissue. The process of decellularization can at least partially remove the cellular portion of the tissue, leaving behind a complex three-dimensional network of extracellular matrix (ECM). An ECM infrastructure can primarily be composed of collagen but can include cytokines, proteoglycans, laminin, fibrillin and other proteins secreted by cells. An at least partially decellularized structure can provide a biocompatible substrate onto which different cell populations can be infused or used to be implanted as acellular medical devices such as but not limited to, wound care matrix, fistula matrix, void filler, dermal fillers, soft tissue reinforcement, or other substrates that enable cellular infiltration and remodeling following implantation or application. Decellularized biostructures can be rigid, or semi-rigid, having an ability to alter their shapes. Examples of decellularized isolated organs can include, but are not limited to solid organs such as, a heart, kidney, liver, lung, pancreas, brain, bone, spleen, and bladder, uterus, ureter, and urethra.

The term “effective amount” or “therapeutically effective amount” can refer to a quantity of a composition, for example a composition comprising cells such as regenerative cells, that can be sufficient to result in a desired activity upon introduction into an isolated organ or portion thereof disclosed herein.

The term “function” and its grammatical equivalents as used herein can refer to a capability of operating, having, or serving an intended purpose. Functional can comprise any percent from baseline to 100% of an intended purpose. For example, functional can comprise or comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional can mean over or over about 100% of normal function, for example, 125%, 150%, 175%, 200%, 250%, 300%, 400%, 500%, 600%, 700% or up to about 1000% of an intended purpose.

The term “recipient” and their grammatical equivalents as used herein can refer to a subject. A subject can be a human or non-human animal. A recipient can also be in need thereof, such as needing treatment for a disease such as cancer. In some embodiments, a recipient can be in need thereof of a preventative therapy. A recipient may not be in need thereof in other cases.

The term “subject” and its grammatical equivalents as used herein can refer to a human or a non-human. A subject can be a mammal. A subject can be a human mammal of a male or female biological gender. A subject can be of any age. A subject can be an embryo. A subject can be a newborn or up to about 100 years of age. A subject can be in need thereof. A subject can have a disease such as cancer. A subject can be premenopausal, menopausal, or have induced menopause.

The terms “treatment” or “treating” and their grammatical equivalents can refer to the medical management of a subject with an intent to cure, ameliorate, or stabilize a disease, condition, or disorder. Treatment can include active treatment, that is, treatment directed specifically toward the improvement of a disease, condition, or disorder. Treatment can include causal treatment, that is, treatment directed toward removal of the cause of the associated disease, condition, or disorder. In addition, this treatment can include palliative treatment, that is, treatment designed for the relief of symptoms rather than the curing of the disease, condition, or disorder. Treatment can include preventative treatment, that is, treatment directed to minimizing or partially or completely inhibiting the development of a disease, condition, or disorder. Treatment can include supportive treatment, that is, treatment employed to supplement another specific therapy directed toward the improvement of the disease, condition, or disorder. In some embodiments, a condition can be pathological. In some embodiments, a treatment may not completely cure, ameliorate, or stabilize a disease, condition, or disorder.

The terms “prevent” or “preventing”, and their grammatical equivalents can refer to prophylactic medical management of a subject. Such prophylactic medical management can include proactive management prior to an incidence of a disease, condition, or disorder, to prevent an onset of the disease, condition, or disorder.

In some embodiments disclosed herein, are compositions and methods of whole organ decellularization as well as decellularization of whole organ portions. Also disclosed herein in some embodiments, are improvements of decellularization utilizing methods and compositions. Also disclosed herein in some embodiments, are compositions and methods of improved decellularization which can result in higher efficiency decellularization and improved recellularization of compositions described herein.

Suitable Organs and Portions Thereof

Disclosed herein in some embodiments, are compositions that can comprise isolated organs or portions thereof. Also disclosed herein in some embodiments, are modified isolated organs or portions thereof. In some embodiments, an isolated organ or portion thereof can be part of an isolated organ system of a body. In some embodiments, isolated organ systems can include without limitation an integumentary, muscular, skeletal, nervous, circulatory, lymphatic, respiratory, endocrine, urinary/excretory, reproductive and digestive systems. In some embodiments, an isolated organ or portion thereof can be from a musculoskeletal system. In some embodiments, isolated organs or portions thereof from a musculoskeletal system can comprise skeleton, joints, ligaments, tendons, muscle. In some embodiments, an isolated organ or portion thereof can be from a digestive system. In some embodiments, a digestive system can comprise a tongue, tooth, salivary gland (parotid, submandibular, sublingual), pharynx, esophagus, stomach, small intestine (duodenum, jejunum, and ileum), large intestine, liver, gall bladder, or pancreas. In some embodiments, an isolated organ or portion thereof can comprise a respiratory system. In some embodiments, a respiratory system can comprise a lung, diaphragm, bronchi, trachea, larynx, pharynx, or nasal cavity. In some embodiments, an isolated organ or portion thereof can comprise a urinary system. In some embodiments, a urinary system can include a kidney, ureter, bladder, or urethra. In some embodiments, an isolated organ or portion thereof can include a reproductive system. In some embodiments, a reproductive system can comprise an ovary, fallopian tube, uterus, vagina, vulva, clitoris, placenta, testes, epididymis, vas deferens, seminal vesicle, prostate, bulbourethral gland, penis, or scrotum. In some embodiments, an isolated organ or portion thereof can include an endocrine gland. In some embodiments, an endocrine gland can comprise a pituitary gland, pineal gland, thyroid gland, parathyroid gland, adrenal gland, or pancreas. In some embodiments, an isolated organ or portion thereof can comprise a circulatory system. In some embodiments, a circulatory system can comprise a cardiovascular system or a lymphatic system. In some embodiments, a cardiovascular system can comprise a heart, artery, vein, or capillary. In some embodiments, a lymphatic system can comprise a lymphatic vessel, a lymph node, bone marrow, thymus, or spleen. In some embodiments, an isolated organ or portion thereof can be from a nervous system. In some embodiments, a nervous system can comprise a central nervous system, peripheral nervous system, or sensory organ. In some embodiments, a central nervous system can comprise a brain, cerebrum, diencephalon, midbrain, pons, medulla oblongata, cerebellum, spinal cord, ventricular system (choroid plexus). In some embodiments, a peripheral nervous system can comprise nerves such as cranial nerves, spinal nerves, ganglia, enteric nervous system. In some embodiments, a sensory organ can comprise an eye (cornea, iris, ciliary body, lens, or retina), ear (earlobe, eardrum, middle ear, cochlea, semicircular canals, and vestibule of the ear), olfactory epithelium, or tongue. In some embodiments, an isolated organ or portion thereof can comprise an integumentary system. In some embodiments, an integumentary system can comprise mammary glands, skin, or subcutaneous tissue. In some embodiments, an isolated organ can be a solid organ. In some embodiments, a solid organ can have a firm tissue consistency and can neither be hollow, such as organs of the gastrointestinal tract, nor liquid, such as blood. In some embodiments, a solid organ can include a heart, kidney, liver, lung, and pancreas. In some embodiments, solid organs can have three main components, an extracellular matrix (ECM), cells embedded therein, and a vasculature bed. In some embodiments, decellularization of a solid organ as described herein can remove most or all cellular components while substantially preserving an extracellular matrix (ECM) and a vasculature bed. In some embodiments, decellularization can comprise decellularizing from about 50%, 60%, 70%, 80%, 85%, 90%, 95%, 96%, 97%, 98%, 99%, or up to about 100% of an isolated organ. In some embodiments, decellularization can comprise decellularizing from about 50%-60%, 50-70%, 60-80%, 60-85%, 70-90%, 70-95%, 75-96%, 75-97%, 80-98%, 80-99%, or up to about 75-100% of an isolated organ.

In some embodiments, an isolated organ or portion thereof can be human. In some embodiments, an isolated organ or portion thereof can be non-human. A method described herein can include providing a perfusion decellularized ECM of a non-human mammalian organ. In some instances, a non-human mammalian organ can be from a large animal. A perfusion decellularized ECM can comprise an intact exterior surface. A perfusion decellularized ECM can comprise a decellularized ECM vascular tree that can include an intact vasculature bed. In some cases, a perfusion decellularized ECM can retain at least a portion of, or a majority of, a fluid introduced to a decellularized ECM vasculature tree.

In some embodiments, an isolated organ or portion thereof can be allogenic, autologous, or xenogeneic. In some embodiments, an isolated organ or portion thereof can be from an adult or pediatric source. In some embodiments, an isolated organ or portion thereof can comprise synthetic components. In some embodiments, non-human isolated organ sources can be from non-human mammals. In some embodiments, non-human isolated organs can be from a non-human primate. In some embodiments, a non-human primate can be a baboon or chimpanzee. In some embodiments, a non-human isolated organ or portion thereof can be from a rodent, pig, rabbit, cattle, sheep, dog, cat, cow, or monkey. In some embodiments, a gnotobiotic mammal can be utilized to source an isolated organ or portion thereof. In some embodiments, a gnotobiotic mammal can be absent a microbial flora. In some embodiments, a gnotobiotic mammal can be surgically delivered under aseptic conditions. In some embodiments, a gnotobiotic mammal can be raised in an isolation type barrier. In some embodiments, a gnotobiotic mammal can be free of microbial flora from about 5%, 10%, 20%, 30%, 40%, 50%, 60%, 70%, 80%, 90%, 95%, 98%, 99%, 100% as compared to an otherwise comparable mammal that is not gnotobiotic. Exemplary zoonotic agents that a gnotobiotic mammal can be substantially devoid of can be rabies, monkey pox virus, Brucella suis, mycobacterium spp, Trypanosoma cruzi, ascaris spp, larvae, Toxoplasma gondii, EMCV, measles, rubella, hepatocystiis kochi, influenza, African swine fever, swine vesicular disease virus, to name a few.

In some embodiments, an isolated organ or portion thereof can be extracted from a non-human animal. In some embodiments, a non-human animal can be a non-human animal that has been fasted. In some embodiments a non-human animal can be a non-human animal that has not been fasted. In some embodiments an animal that has not been fasted can be a non-human animal that has been fed within about 12 hours, about 10 hours, about 8 hours, about 6 hours, about 4 hours, about 2 hours, or about 1 hour prior to removal of the isolated organ or portion thereof from the animal.

An isolated organ or portion thereof can comprise a reduced particulate level prior to, during, or after decellularization. For instance, a method of decellularization as described herein can proceed with a lower occurrence of particulates. Accordingly, a decellularization process can proceed e.g. faster, more efficiently, to a greater extent, etc., relative to an otherwise comparable decellularization method with a higher occurrence of particulates.

In some embodiments, an isolated organ or portion thereof can comprise a reduced particulate level as compared to an otherwise comparable isolated organ or portion thereof produced from an animal which had not been fed within about 12 hours, about 10 hours, about 8 hours, about 6 hours, about 4 hours, about 2 hours, or about 1 hour prior to removal of an isolated organ or portion thereof from the animal. In some embodiments, a reduced particulate level can be reduced by at least about: 90%, 85%, 80%, 75%, 70%, 65%, 60%, 55%, 50%, 45%, 40%, 35%, 30%, 25%, 20%, 15%, 10%, 5%, 1% as compared to an otherwise comparable isolated organ or portion thereof produced from an animal which had not been fed within about 12 hours, about 10 hours, about 8 hours, about 6 hours, about 4 hours, about 2 hours, or about 1 hour prior to removal of the isolated organ or portion thereof from the animal.

Organ Decellularization

Also disclosed herein in some embodiments, are compositions that can comprise decellularized isolated organs and methods of decellularization. In some embodiments, an isolated organ can undergo decellularization. In some embodiments, decellularization can refer to a process comprising isolating an extracellular matrix (ECM) of a tissue from its native cells. An at least partially decellularized isolated organ or tissue can comprise an extracellular matrix (ECM) component of all or most regions of an isolated organ or portion thereof, or tissue, including ECM components of a vascular tree. In some embodiments, an ECM component can include: fibronectin, fibrillin, laminin, elastin, members of the collagen family (e.g., collagen I, III, and IV), glycosaminoglycans, proteoglycans, cytokines, heparin sulfate, ground substance, reticular fibers or thrombospondin, or any combination thereof, which can remain organized as defined structures such as a basal lamina. In some embodiments, decellularization can refer to a reduction or absence of detectable myofilaments, endothelial cells, smooth muscle cells, and nuclei in histologic sections using standard histological staining procedures. Residual cell debris can also be removed from a decellularized isolated organ or tissue. Organ decellularization can be performed using a variety of techniques such as perfusion decellularization, immersion decellularization, physical treatments, chemical treatments, enzymatic treatments, and combinations thereof.

Perfusion Decellularization

Also disclosed herein in some embodiments, are methods that can comprise perfusion-based decellularization of a solid organ or portion thereof. In some embodiments, perfusion decellularization can allow for decellularization through a native vasculature, ducts, cavities, and combinations thereof which can result in a rapid decellularization of large organs or portions thereof while maintaining vascular conduits critical to re-engineering of tissue intact. In some embodiments, perfusion decellularization can provide for rapid access to a whole organ through a native vasculature by cannulating a vasculature and perfusing a mild detergent solution through a native blood vessel. Because organs can be dense with vascular capillaries, most cells can be located within 50-100 micrometers (μm) of a capillary, resulting in an exponential increase in effective surface area of a detergent and decreased time to dissolve cellular material during expulsion through a venous system, as opposed to through an isolated organ wall or capsule as can be done in immersion-based decellularization. Perfusion decellularization of an ECM from an isolated organ or tissue can retain a native microstructure, such as an intact vascular and/or microvascular system, as compared to other decellularization techniques such as immersion based decellularization. For example, perfusion decellularized ECM from organs or tissues can preserve collagen content and other binding and signaling factors and vasculature structure, thus providing for a niche environment with native cues for functional differentiation or maintenance of cellular function of introduced cells. In some embodiments, perfusion decellularized ECM from organs or tissues can be perfused with cells and/or media using a vasculature of a perfusion decellularized ECM under appropriate conditions, including appropriate pressure and flow to mimic conditions normally found in an organism. A normal pressure of human sized organs can be between about 5 mm Hg to about 200 mm Hg with a resulting flow rate dependent upon an incoming perfusion vessel diameter. In some embodiments, a normal pressure of human sized organs can be from 5 mm Hg, 10 mm Hg, 15 mm Hg, 20 mmHg, 25 mmHg, 30 mmHg, 40 mmHg, 50 mmHg, 60 mmHg, 80 mmHg, 100 mmHg, 120 mmHg, 140 mmHg, 160 mmHg, 180 mmHg, or up to about 200 mmHg. For a normal human heart, a resulting perfusion flow can be about 20 mL/min/100 g to about 200 mL/min/100 g. Using such a system for seeded cells can achieve a greater seeding concentration of, for example, about 5× up to about 1000× greater than achieved under 2D cell culture conditions and, unlike a 2D culture system, an ECM environment allows for a further functional differentiation of cells, e.g., differentiation of progenitor cells into cells that demonstrate organ- or tissue-specific phenotypes having sustained function. Perfusion decellularization can comprise cannulating an isolated organ or portion thereof. In some embodiments, at least one cannulation can be introduced to an isolated organ or portion thereof. In some cases, at least two cannulations can be introduced to an isolated organ or portion thereof. In some embodiments, from about 1, 2, 3, 4, 5, 6, 7, 8, 9, or up to 10 cannulations can be introduced to an isolated organ or portion thereof. In some embodiments, a cannula can be a part of a cannulation system. In some embodiments, a cannulation system can comprise a size-appropriate hollow tubing for introducing into a vessel, duct, cavity, or any combination thereof of an isolated tissue, organ or portion thereof. In some embodiments, a cannula can be of an animal-appropriate diameter; for example, a cannula can be of a certain gauge. In some embodiments, gauges can range from 1 to 75. In some embodiments, a gauge can be of gauge 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75. In some embodiments, a cannula can have a gauge of about 16. In some embodiments, a cannula can have a gauge of about 23. In some embodiments, a cannula can have a gauge of about 27. In some embodiments, a cannula can have a gauge of about 30. In some embodiments, a cannula can have a gauge of about 34. In some embodiments, a cannula can have a gauge between 16 gauge and 34 gauge. In some embodiments, a cannula can have a gauge between 30 gauge and 50 gauge. In some embodiments, a cannula can have a gauge between 30 gauge and 50 gauge. In some embodiments, a type of “cannula” can vary. In some embodiments, a typical cannula used for blood draw or IV can be ˜14-26 gauge. In some embodiments, a cannula can comprise a barbed connector with a luer fitting or a two-way barbed tube to tube connector. In some embodiments, a barbed end can vary from about 1/32″ to about ⅝″ and greater; for example, a cannula size for a 6 month porcine liver can be ⅝″ diameter. In some embodiments, a pig can use a cannula from 1/16″ to about ¼″. In some embodiments, a cannula can have a gauge from about 1/16″ to about 1″. In some embodiments, an isolated organ or portion thereof can comprise a cannula of a gauge of at least 34. In some embodiments, disclosed herein can be an isolated organ or portion thereof comprising a cannula of a gauge of at least 50. In some embodiments, disclosed herein can be an isolated organ or portion thereof comprising a cannula of a gauge of at least 16. In some embodiments, disclosed herein can be an isolated organ or portion thereof comprising a cannula of a gauge of at least 20. In some embodiments, at least one vessel, duct, and/or cavity can be cannulated in an isolated organ. In some embodiments, a perfusion apparatus or cannulation system can include a holding container for solutions (e.g., a cellular disruption medium) and a mechanism for moving a liquid through an isolated organ (e.g., a pump, air pressure, gravity) via one or more cannulae. In some embodiments, an isolated organ or tissue during decellularization and/or recellularization can be maintained in a sterile state using a variety of techniques such as: controlling and filtering air flow and/or perfusing with, for example, antibiotics, anti-fungals or other anti-microbials to prevent growth of unwanted microorganisms. In some embodiments, a system as described herein can possess an ability to monitor certain perfusion characteristics (e.g., pressure, volume, flow pattern, temperature, gases, pH), mechanical forces (e.g., ventricular wall motion and stress), and electrical stimulation (e.g., pacing). In some embodiments, a vascular bed can change during decellularization and recellularization (e.g., vascular resistance, volume). In some embodiments, a pressure-regulated perfusion apparatus or cannulation system can be advantageous to avoid or reduce fluctuations. In some embodiments, an effectiveness of perfusion can be evaluated in an effluent and in tissue sections. In some embodiments, perfusion volume, flow pattern, temperature, partial O₂ and CO₂ pressures and pH can be monitored using standard methods. In some embodiments, sensors can be used to monitor a system (e.g., a bioreactor) and/or an isolated tissue, organ or portion thereof. In some embodiments, sonomicrometry, micromanometry, and/or conductance measurements can be used to acquire pressure-volume or preload recruitable stroke work information relative to myocardial wall motion and performance. In some embodiments, for example, sensors can be used to monitor a pressure of a liquid moving through a cannulated organ or tissue; an ambient temperature in a system and/or a temperature of an isolated organ or tissue; a pH and/or a rate of flow of a liquid moving through a cannulated organ or tissue; and/or a biological activity of a recellularizing organ or tissue. In some embodiments, in addition to having sensors for monitoring such features, a system for decellularizing and/or recellularizing an isolated organ or tissue also can include means for maintaining or adjusting such features. In some embodiments, means for maintaining or adjusting such features can include components such as a thermometer, a thermostat, electrodes, pressure sensors, overflow valves, valves for changing a rate of flow of a liquid, valves for opening and closing fluid connections to solutions used for changing a pH of a solution, a balloon, an external pacemaker, and/or a compliance chamber. In some embodiments, to help ensure stable conditions (e.g., temperature), a chamber, reservoir, or tubing can be water-jacketed. In some embodiments, a method of decellularization can comprise providing an isolated organ or portion thereof, cannulating an isolated organ or portion thereof, and perfusing a cannulated isolated organ or portion thereof with a solution or medium via a cannulation. In some embodiments, a cannulation occurs at a cavity, vessel, duct, or combination thereof. In some embodiments, from about 1 to 3, from about 1 to 5, from about 2 to 3, from about 2 to 5, from about 1 to 8 solutions can be utilized for organ perfusion. In some embodiments, a solution can be perfused at least two times. In some embodiments, a solution can be perfused at least 3, 4, 5, 6, 7, 8, 9, or up to 10 times through an isolated organ or portion thereof. In some embodiments, various solutions and mediums can be employed during recellularization. In some embodiments, a solution can be selected from a group comprising: cellular disruption media, washing solutions, disinfecting solutions, or combinations thereof. In some embodiments, a cellular disruption media can be a solution that can comprise at least one detergent, Table 1. In some embodiments, at least one of: Sodium dodecyl sulfate (SDS), Polyethylene glycol p-(1,1,3,3-tetramethylbutyl)-phenyl ether (Triton X), NP-40, Brij, Polyoxyethylene sorbitan monolaurate (Tween), Octyl glucoside, octyl thioglucoside, 3-[(3-cholamidopropyl)dimethylammonio]-1-propanesulfonate (CHAPS), 3-[(3-Cholamidopropyl)dimethylammonio]-2-hydroxy-1-propanesulfonate (CHAPSO), salts thereof, or modified versions thereof can be utilized during decellularization. In some embodiments, a detergent can be an amphipathic molecule that can contain both a nonpolar “tail” having aliphatic or aromatic character and a polar “head”. In some embodiments, an ionic character of a polar head group can form a basis for broad classification of detergents; they can be ionic (charged, either anionic or cationic), nonionic (uncharged), or zwitterionic (having both positively and negatively charged groups but with a net charge of zero). In some embodiments, detergents can be denaturing or non-denaturing with respect to protein structure. In some embodiments, denaturing detergents can be anionic such as sodium dodecyl sulfate (SDS) or cationic such as ethyl trimethyl ammonium bromide (ETMAB). In some embodiments, these detergents totally disrupt membranes and denature proteins by breaking protein-protein interactions. In some embodiments, non-denaturing detergents can be divided into nonionic detergents such as Triton X-100, NP40, Tween, bile salts such as cholate, and zwitterionic detergents such as CHAPS. In some embodiments, an at least partially degassed liquid solution can be utilized during decellularization. In some embodiments, a washing solution can be utilized to remove residual solutions such as cellular disruption media from an isolated organ or portion thereof as well as residual cellular components, enzymes, or combinations thereof. In some embodiments, suitable washing solutions can comprise water, filtered water, normal saline, phosphate buffered saline (PBS), or any combination thereof. In some embodiments, PBS can maintain a constant pH and constant osmolarity of cells (a pH of most biological materials falls from about 7 to about 7.6). In some embodiments, any concentration of PBS can be utilized as a washing solution. In some embodiments, a concentration of PBS can be about 0.5%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 35%, 40%, 45%, 50%, 55%, 60%, 65%, 70%, 75%, 80%, 85%, 90%, 95%, or up to about 100%. In some embodiments, a washing solution can be supplemented with agents. In some embodiments, an agent can be an antibiotic, DNase I, or a disinfectant. In some embodiments, a washing solution can comprise saline. In some embodiments, a washing solution can be 0.9% NaCl, 0.55% PBS, and 100% PBS, saline, and any combination thereof. In some embodiments, a cellular disruption media can be a degassed cellular disruption media. In some embodiments, a “degassed” cellular disruption media can refer to a disruption media as described herein, where a dissolved gas has been substantially removed. In some embodiments, a dissolved gas can include oxygen, nitrogen, air, carbon dioxide, carbon monoxide, argon, hydrogen sulfide, methane, ethylene, ethane, chlorine, hydrogen, helium, ammonia, sulfur dioxide, or any combination thereof. In some embodiments, a degassed media can contain less dissolved gases than a non-degassed media. In some embodiments, a degassed media can contain about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, 49.5%, 50%, 50.5%, 51%, 51.5%, 52%, 52.5%, 53%, 53.5%, 54%, 54.5%, 55%, 55.5%, 56%, 56.5%, 57%, 57.5%, 58%, 58.5%, 59%, 59.5%, 60%, 60.5%, 61%, 61.5%, 62%, 62.5%, 63%, 63.5%, 64%, 64.5%, 65%, 65.5%, 66%, 66.5%, 67%, 67.5%, 68%, 68.5%, 69%, 69.5%, 70%, 70.5%, 71%, 71.5%, 72%, 72.5%, 73%, 73.5%, 74%, 74.5%, 75%, 75.5%, 76%, 76.5%, 77%, 77.5%, 78%, 78.5%, 79%, 79.5%, 80%, 80.5%, 81%, 81.5%, 82%, 82.5%, 83%, 83.5%, 84%, 84.5%, 85%, 85.5%, 86%, 86.5%, 87%, 87.5%, 88%, 88.5%, 89%, 89.5%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 99.9% of a dissolved gas content of a non-degassed media. In some embodiments, a degassed media can contain an amount of a gas at a given temperature; for example, a degassed media can contain less than about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 20.1, 20.2, 20.3, 20.4, 20.5, 20.6, 20.7, 20.8, 20.9, 21, 21.1, 21.2, 21.3, 21.4, 21.5, 21.6, 21.7, 21.8, 21.9, 22, 22.1, 22.2, 22.3, 22.4, 22.5, 22.6, 22.7, 22.8, 22.9, 23, 23.1, 23.2, 23.3, 23.4, 23.5, 23.6, 23.7, 23.8, 23.9, 24, 24.1, 24.2, 24.3, 24.4, 24.5, 24.6, 24.7, 24.8, 24.9, 25, 25.1, 25.2, 25.3, 25.4, 25.5, 25.6, 25.7, 25.8, 25.9, 26, 26.1, 26.2, 26.3, 26.4, 26.5, 26.6, 26.7, 26.8, 26.9, 27, 27.1, 27.2, 27.3, 27.4, 27.5, 27.6, 27.7, 27.8, 27.9, 28, 28.1, 28.2, 28.3, 28.4, 28.5, 28.6, 28.7, 28.8, 28.9, 29, 29.1, 29.2, 29.3, 29.4, 29.5, 29.6, 29.7, 29.8, 29.9, 30, 30.1, 30.2, 30.3, 30.4, 30.5, 30.6, 30.7, 30.8, 30.9, 31, 31.1, 31.2, 31.3, 31.4, 31.5, 31.6, 31.7, 31.8, 31.9, 32, 32.1, 32.2, 32.3, 32.4, 32.5, 32.6, 32.7, 32.8, 32.9, 33, 33.1, 33.2, 33.3, 33.4, 33.5, 33.6, 33.7, 33.8, 33.9, 34, 34.1, 34.2, 34.3, 34.4, 34.5, 34.6, 34.7, 34.8, 34.9, 35, 35.1, 35.2, 35.3, 35.4, 35.5, 35.6, 35.7, 35.8, 35.9, 36, 36.1, 36.2, 36.3, 36.4, 36.5, 36.6, 36.7, 36.8, 36.9, 37, 37.1, 37.2, 37.3, 37.4, 37.5, 37.6, 37.7, 37.8, 37.9, 38, 38.1, 38.2, 38.3, 38.4, 38.5, 38.6, 38.7, 38.8, 38.9, 39, 39.1, 39.2, 39.3, 39.4, 39.5, 39.6, 39.7, 39.8, 39.9, 40, 40.1, 40.2, 40.3, 40.4, 40.5, 40.6, 40.7, 40.8, 40.9, 41, 41.1, 41.2, 41.3, 41.4, 41.5, 41.6, 41.7, 41.8, 41.9, 42, 42.1, 42.2, 42.3, 42.4, 42.5, 42.6, 42.7, 42.8, 42.9, 43, 43.1, 43.2, 43.3, 43.4, 43.5, 43.6, 43.7, 43.8, 43.9, 44, 44.1, 44.2, 44.3, 44.4, 44.5, 44.6, 44.7, 44.8, 44.9, 45, 45.1, 45.2, 45.3, 45.4, 45.5, 45.6, 45.7, 45.8, 45.9, 46, 46.1, 46.2, 46.3, 46.4, 46.5, 46.6, 46.7, 46.8, 46.9, 47, 47.1, 47.2, 47.3, 47.4, 47.5, 47.6, 47.7, 47.8, 47.9, 48, 48.1, 48.2, 48.3, 48.4, 48.5, 48.6, 48.7, 48.8, 48.9, 49, 49.1, 49.2, 49.3, 49.4, 49.5, 49.6, 49.7, 49.8, 49.9, 50, 50.1, 50.2, 50.3, 50.4, 50.5, 50.6, 50.7, 50.8, 50.9, 51, 51.1, 51.2, 51.3, 51.4, 51.5, 51.6, 51.7, 51.8, 51.9, 52, 52.1, 52.2, 52.3, 52.4, 52.5, 52.6, 52.7, 52.8, 52.9, 53, 53.1, 53.2, 53.3, 53.4, 53.5, 53.6, 53.7, 53.8, 53.9, 54, 54.1, 54.2, 54.3, 54.4, 54.5, 54.6, 54.7, 54.8, 54.9, 55, 55.1, 55.2, 55.3, 55.4, 55.5, 55.6, 55.7, 55.8, 55.9, 56, 56.1, 56.2, 56.3, 56.4, 56.5, 56.6, 56.7, 56.8, 56.9, 57, 57.1, 57.2, 57.3, 57.4, 57.5, 57.6, 57.7, 57.8, 57.9, 58, 58.1, 58.2, 58.3, 58.4, 58.5, 58.6, 58.7, 58.8, 58.9, 59, 59.1, 59.2, 59.3, 59.4, 59.5, 59.6, 59.7, 59.8, 59.9, 60, 60.1, 60.2, 60.3, 60.4, 60.5, 60.6, 60.7, 60.8, 60.9, 61, 61.1, 61.2, 61.3, 61.4, 61.5, 61.6, 61.7, 61.8, 61.9, 62, 62.1, 62.2, 62.3, 62.4, 62.5, 62.6, 62.7, 62.8, 62.9, 63, 63.1, 63.2, 63.3, 63.4, 63.5, 63.6, 63.7, 63.8, 63.9, 64, 64.1, 64.2, 64.3, 64.4, 64.5, 64.6, 64.7, 64.8, 64.9, 65, 65.1, 65.2, 65.3, 65.4, 65.5, 65.6, 65.7, 65.8, 65.9, 66, 66.1, 66.2, 66.3, 66.4, 66.5, 66.6, 66.7, 66.8, 66.9, 67, 67.1, 67.2, 67.3, 67.4, 67.5, 67.6, 67.7, 67.8, 67.9, 68, 68.1, 68.2, 68.3, 68.4, 68.5, 68.6, 68.7, 68.8, 68.9, 69, 69.1, 69.2, 69.3, 69.4, 69.5, 69.6, 69.7, 69.8, 69.9, 70, 70.1, 70.2, 70.3, 70.4, 70.5, 70.6, 70.7, 70.8, 70.9, 71, 71.1, 71.2, 71.3, 71.4, 71.5, 71.6, 71.7, 71.8, 71.9, 72, 72.1, 72.2, 72.3, 72.4, 72.5, 72.6, 72.7, 72.8, 72.9, 73, 73.1, 73.2, 73.3, 73.4, 73.5, 73.6, 73.7, 73.8, 73.9, 74, 74.1, 74.2, 74.3, 74.4, 74.5, 74.6, 74.7, 74.8, 74.9, 75, 75.1, 75.2, 75.3, 75.4, 75.5, 75.6, 75.7, 75.8, 75.9, 76, 76.1, 76.2, 76.3, 76.4, 76.5, 76.6, 76.7, 76.8, 76.9, 77, 77.1, 77.2, 77.3, 77.4, 77.5, 77.6, 77.7, 77.8, 77.9, 78, 78.1, 78.2, 78.3, 78.4, 78.5, 78.6, 78.7, 78.8, 78.9, 79, 79.1, 79.2, 79.3, 79.4, 79.5, 79.6, 79.7, 79.8, 79.9, 80, 80.1, 80.2, 80.3, 80.4, 80.5, 80.6, 80.7, 80.8, 80.9, 81, 81.1, 81.2, 81.3, 81.4, 81.5, 81.6, 81.7, 81.8, 81.9, 82, 82.1, 82.2, 82.3, 82.4, 82.5, 82.6, 82.7, 82.8, 82.9, 83, 83.1, 83.2, 83.3, 83.4, 83.5, 83.6, 83.7, 83.8, 83.9, 84, 84.1, 84.2, 84.3, 84.4, 84.5, 84.6, 84.7, 84.8, 84.9, 85, 85.1, 85.2, 85.3, 85.4, 85.5, 85.6, 85.7, 85.8, 85.9, 86, 86.1, 86.2, 86.3, 86.4, 86.5, 86.6, 86.7, 86.8, 86.9, 87, 87.1, 87.2, 87.3, 87.4, 87.5, 87.6, 87.7, 87.8, 87.9, 88, 88.1, 88.2, 88.3, 88.4, 88.5, 88.6, 88.7, 88.8, 88.9, 89, 89.1, 89.2, 89.3, 89.4, 89.5, 89.6, 89.7, 89.8, 89.9, 90, 90.1, 90.2, 90.3, 90.4, 90.5, 90.6, 90.7, 90.8, 90.9, 91, 91.1, 91.2, 91.3, 91.4, 91.5, 91.6, 91.7, 91.8, 91.9, 92, 92.1, 92.2, 92.3, 92.4, 92.5, 92.6, 92.7, 92.8, 92.9, 93, 93.1, 93.2, 93.3, 93.4, 93.5, 93.6, 93.7, 93.8, 93.9, 94, 94.1, 94.2, 94.3, 94.4, 94.5, 94.6, 94.7, 94.8, 94.9, 95, 95.1, 95.2, 95.3, 95.4, 95.5, 95.6, 95.7, 95.8, 95.9, 96, 96.1, 96.2, 96.3, 96.4, 96.5, 96.6, 96.7, 96.8, 96.9, 97, 97.1, 97.2, 97.3, 97.4, 97.5, 97.6, 97.7, 97.8, 97.9, 98, 98.1, 98.2, 98.3, 98.4, 98.5, 98.6, 98.7, 98.8, 98.9, 99, 99.1, 99.2, 99.3, 99.4, 99.5, 99.6, 99.7, 99.8, 99.9, or 100 ppb of a gas at 25° C. In some embodiments, a degassed media can contain less than about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10 ppm of a gas at 25° C. In some embodiments, a degassed media can be prepared by placing a media under reduced pressure (e.g. under a vacuum or a vacuum pump). In some embodiments, a “reduced pressure” can be a pressure less than an atmospheric pressure; for instance, a “reduced pressure” can be a pressure less than 760 torr. In some embodiments, a reduced pressure can be a vacuum, a medium vacuum, a high vacuum, or an ultra-high vacuum. In some embodiments, a reduced pressure can be a pressure less than about 760, 759, 758, 757, 756, 755, 754, 753, 752, 751, 750, 749, 748, 747, 746, 745, 744, 743, 742, 741, 740, 739, 738, 737, 736, 735, 734, 733, 732, 731, 730, 729, 728, 727, 726, 725, 724, 723, 722, 721, 720, 719, 718, 717, 716, 715, 714, 713, 712, 711, 710, 709, 708, 707, 706, 705, 704, 703, 702, 701, 700, 699, 698, 697, 696, 695, 694, 693, 692, 691, 690, 689, 688, 687, 686, 685, 684, 683, 682, 681, 680, 679, 678, 677, 676, 675, 674, 673, 672, 671, 670, 669, 668, 667, 666, 665, 664, 663, 662, 661, 660, 659, 658, 657, 656, 655, 654, 653, 652, 651, 650, 649, 648, 647, 646, 645, 644, 643, 642, 641, 640, 639, 638, 637, 636, 635, 634, 633, 632, 631, 630, 629, 628, 627, 626, 625, 624, 623, 622, 621, 620, 619, 618, 617, 616, 615, 614, 613, 612, 611, 610, 609, 608, 607, 606, 605, 604, 603, 602, 601, 600, 599, 598, 597, 596, 595, 594, 593, 592, 591, 590, 589, 588, 587, 586, 585, 584, 583, 582, 581, 580, 579, 578, 577, 576, 575, 574, 573, 572, 571, 570, 569, 568, 567, 566, 565, 564, 563, 562, 561, 560, 559, 558, 557, 556, 555, 554, 553, 552, 551, 550, 549, 548, 547, 546, 545, 544, 543, 542, 541, 540, 539, 538, 537, 536, 535, 534, 533, 532, 531, 530, 529, 528, 527, 526, 525, 524, 523, 522, 521, 520, 519, 518, 517, 516, 515, 514, 513, 512, 511, 510, 509, 508, 507, 506, 505, 504, 503, 502, 501, 500, 499, 498, 497, 496, 495, 494, 493, 492, 491, 490, 489, 488, 487, 486, 485, 484, 483, 482, 481, 480, 479, 478, 477, 476, 475, 474, 473, 472, 471, 470, 469, 468, 467, 466, 465, 464, 463, 462, 461, 460, 459, 458, 457, 456, 455, 454, 453, 452, 451, 450, 449, 448, 447, 446, 445, 444, 443, 442, 441, 440, 439, 438, 437, 436, 435, 434, 433, 432, 431, 430, 429, 428, 427, 426, 425, 424, 423, 422, 421, 420, 419, 418, 417, 416, 415, 414, 413, 412, 411, 410, 409, 408, 407, 406, 405, 404, 403, 402, 401, 400, 399, 398, 397, 396, 395, 394, 393, 392, 391, 390, 389, 388, 387, 386, 385, 384, 383, 382, 381, 380, 379, 378, 377, 376, 375, 374, 373, 372, 371, 370, 369, 368, 367, 366, 365, 364, 363, 362, 361, 360, 359, 358, 357, 356, 355, 354, 353, 352, 351, 350, 349, 348, 347, 346, 345, 344, 343, 342, 341, 340, 339, 338, 337, 336, 335, 334, 333, 332, 331, 330, 329, 328, 327, 326, 325, 324, 323, 322, 321, 320, 319, 318, 317, 316, 315, 314, 313, 312, 311, 310, 309, 308, 307, 306, 305, 304, 303, 302, 301, 300, 299, 298, 297, 296, 295, 294, 293, 292, 291, 290, 289, 288, 287, 286, 285, 284, 283, 282, 281, 280, 279, 278, 277, 276, 275, 274, 273, 272, 271, 270, 269, 268, 267, 266, 265, 264, 263, 262, 261, 260, 259, 258, 257, 256, 255, 254, 253, 252, 251, 250, 249, 248, 247, 246, 245, 244, 243, 242, 241, 240, 239, 238, 237, 236, 235, 234, 233, 232, 231, 230, 229, 228, 227, 226, 225, 224, 223, 222, 221, 220, 219, 218, 217, 216, 215, 214, 213, 212, 211, 210, 209, 208, 207, 206, 205, 204, 203, 202, 201, 200, 199, 198, 197, 196, 195, 194, 193, 192, 191, 190, 189, 188, 187, 186, 185, 184, 183, 182, 181, 180, 179, 178, 177, 176, 175, 174, 173, 172, 171, 170, 169, 168, 167, 166, 165, 164, 163, 162, 161, 160, 159, 158, 157, 156, 155, 154, 153, 152, 151, 150, 149, 148, 147, 146, 145, 144, 143, 142, 141, 140, 139, 138, 137, 136, 135, 134, 133, 132, 131, 130, 129, 128, 127, 126, 125, 124, 123, 122, 121, 120, 119, 118, 117, 116, 115, 114, 113, 112, 111, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 tor-. In some embodiments, a reduced pressure can be a pressure less than about 1×10⁰, 1×10⁻¹, 1×10⁻², 1×10⁻³, 1×10⁻⁴, 1×10⁻⁵, 1×10⁻⁶, 1×10⁻⁷, 1×10⁻⁸, 1×10⁻⁹ 1×10⁻¹⁰, 1×10⁻¹¹, or 1×10⁻¹² torr. In some embodiments, a degassed media can be prepared by heating a degassed media. In some embodiments, a degassed media can be placed at a temperature of about 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C. In some embodiments, a degassed media can be placed at a temperature of from about 25° C. to about 27° C., from about 25° C. to about 28° C., from about 25° C. to about 29° C., from about 25° C. to about 30° C., from about 25° C. to about 31° C., from about 25° C. to about 32° C., from about 25° C. to about 33° C., from about 25° C. to about 34° C., from about 25° C. to about 35° C., from about 25° C. to about 36° C., from about 25° C. to about 37° C., from about 25° C. to about 38° C., from about 25° C. to about 39° C., from about 25° C. to about 40° C., from about 25° C. to about 41° C., from about 25° C. to about 42° C., from about 25° C. to about 43° C., from about 25° C. to about 44° C., from about 25° C. to about 45° C., from about 25° C. to about 46° C., from about 25° C. to about 47° C., from about 25° C. to about 48° C., from about 25° C. to about 49° C., from about 25° C. to about 50° C., from about 25° C. to about 51° C., from about 25° C. to about 52° C., from about 25° C. to about 53° C., from about 25° C. to about 54° C., from about 25° C. to about 55° C., from about 25° C. to about 56° C., from about 25° C. to about 57° C., from about 25° C. to about 58° C., from about 25° C. to about 59° C., from about 25° C. to about 60° C., from about 25° C. to about 61° C., from about 25° C. to about 62° C., from about 25° C. to about 63° C., from about 25° C. to about 64° C., from about 25° C. to about 65° C., from about 25° C. to about 66° C., from about 25° C. to about 67° C., from about 25° C. to about 68° C., from about 25° C. to about 69° C., from about 25° C. to about 70° C., from about 25° C. to about 71° C., from about 25° C. to about 72° C., from about 25° C. to about 73° C., from about 25° C. to about 74° C., from about 25° C. to about 75° C., from about 25° C. to about 76° C., from about 25° C. to about 77° C., from about 25° C. to about 78° C., from about 25° C. to about 79° C., from about 25° C. to about 80° C., from about 25° C. to about 81° C., from about 25° C. to about 82° C., from about 25° C. to about 83° C., from about 25° C. to about 84° C., from about 25° C. to about 85° C., from about 25° C. to about 86° C., from about 25° C. to about 87° C., from about 25° C. to about 88° C., from about 25° C. to about 89° C., from about 25° C. to about 90° C., from about 25° C. to about 91° C., from about 25° C. to about 92° C., from about 25° C. to about 93° C., from about 25° C. to about 94° C., from about 25° C. to about 95° C., from about 25° C. to about 96° C., from about 25° C. to about 97° C., from about 25° C. to about 98° C., from about 25° C. to about 99° C., from about 25° C. to about 100° C. In some embodiments, a degassed media can be placed at a temperature of from about 50° C. to about 52° C., from about 50° C. to about 53° C., from about 50° C. to about 54° C., from about 50° C. to about 55° C., from about 50° C. to about 56° C., from about 50° C. to about 57° C., from about 50° C. to about 58° C., from about 50° C. to about 59° C., from about 50° C. to about 60° C., from about 50° C. to about 61° C., from about 50° C. to about 62° C., from about 50° C. to about 63° C., from about 50° C. to about 64° C., from about 50° C. to about 65° C., from about 50° C. to about 66° C., from about 50° C. to about 67° C., from about 50° C. to about 68° C., from about 50° C. to about 69° C., from about 50° C. to about 70° C., from about 50° C. to about 71° C., from about 50° C. to about 72° C., from about 50° C. to about 73° C., from about 50° C. to about 74° C., from about 50° C. to about 75° C., from about 50° C. to about 76° C., from about 50° C. to about 77° C., from about 50° C. to about 78° C., from about 50° C. to about 79° C., from about 50° C. to about 80° C., from about 50° C. to about 81° C., from about 50° C. to about 82° C., from about 50° C. to about 83° C., from about 50° C. to about 84° C., from about 50° C. to about 85° C., from about 50° C. to about 86° C., from about 50° C. to about 87° C., from about 50° C. to about 88° C., from about 50° C. to about 89° C., from about 50° C. to about 90° C., from about 50° C. to about 91° C., from about 50° C. to about 92° C., from about 50° C. to about 93° C., from about 50° C. to about 94° C., from about 50° C. to about 95° C., from about 50° C. to about 96° C., from about 50° C. to about 97° C., from about 50° C. to about 98° C., from about 50° C. to about 99° C., or from about 50° C. to about 100° C. In some embodiments, a media can be placed under reduced pressure, under elevated pressure, or both, for a given amount of time to reduce a concentration of dissolved gas. In some embodiments, a media can be placed under reduced pressure, under elevated pressure, or both for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes. In some embodiments, a media can be placed under reduced pressure, under elevated pressure, or both for at least 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, or 24 hours. In some embodiments, a degassed media (e.g. a degassed decellularization or recellularization media) can perform more efficiently than a non-degassed media when perfused into an isolated organ; for example, decellularization using a degassed media can require less volume of a degassed media relative to a non-degassed media to achieve a comparable amount of decellularization. In some embodiments, decellularization using a degassed media can require less time to achieve a given amount of decellularization relative to an amount of time required to achieve a comparable amount of decellularization using a non-degassed media. In some embodiments, perfusion with a degassed media produces fewer microbubbles than perfusion with a non-degassed media. In some embodiments, a disinfecting solution can be utilized during decellularization. In some embodiments, a disinfecting solution can comprise any number of agents such as antibiotics, disinfectants, or combinations thereof. In some embodiments, an antibiotic that can be used in a decellularization solution can be selected from a group comprising: actinomycin, ampicillin, carbenicillin, cefotaxime, fosmidomycin, gentamicin, kanamycin, neomycin, amphotericin, penicillin, polymyxin, streptomycin, broad selection antibiotic, and combinations thereof. In some embodiments, any concentration of antibiotic can be introduced in a disinfecting solution. In some embodiments, suitable concentrations of antibiotics can be: 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 35%, 40%, 45%, 50%, or up to about 60%. In some embodiments, suitable concentrations of antibiotics can be: 0.5 U/ml, 1 U/ml, 5 U/ml, 10 U/ml, 20 U/ml, 30 U/ml, 40 U/ml, 50 U/ml, 60 U/ml, 70 U/ml, 80 U/ml, 90 U/ml, 100 U/ml, 110 U/ml, 120 U/ml, 130 U/ml, 140 U/ml, 150 U/ml, 160 U/ml, 170 U/ml, 180 U/ml, 190 U/ml, 200 U/ml, 300 U/ml, 400 U/ml, 500 U/ml, 600 U/ml, 700 U/ml, 800 U/ml, 900 U/ml, 1000 U/ml, and up to about 1500 U/ml. In some embodiments, suitable concentrations of antibiotics can be: 0.5 μg/ml, 1 μg/ml, 1.5 μg/ml, 2 μg/ml, 2.5 μg/ml, 3 μg/ml, 3.5 μg/ml, 4 μg/ml, 4.5 μg/ml, 5 μg/ml, 5.5 μg/ml, 6 μg/ml, 6.5 μg/ml, 7 μg/ml, 7.5 μg/ml, 8 μg/ml, 8.5 μg/ml, 9 μg/ml, 9.5 μg/ml, 10 μg/ml, 15 μg/ml, 20 μg/ml, 25 μg/ml, 30 μg/ml, 35 μg/ml, 40 μg/ml, 45 μg/ml, 50 μg/ml, or up to about 60 μg/ml. In some embodiments, an antibiotic can be 1% benzalkonium chloride, 100 U/ml penicillin-G, 100 U/ml streptomycin, 50 μg/ml Gentamicin and 0.25 μg/ml Amphotericin B. In some embodiments, generally, moderate concentrations of mild (i.e., nonionic) detergents can compromise cell membrane integrity, thereby facilitating lysis of cells and extraction of soluble protein, often in native form. In some embodiments, using certain buffer conditions, various detergents effectively penetrate between a membrane bilayer at a concentration sufficient to form mixed micelles with isolated phospholipids and membrane proteins. In some embodiments, denaturing detergents such as SDS can bind to both membrane (hydrophobic) and non-membrane (water-soluble, hydrophilic) proteins at concentrations below a CMC (i.e., as monomers). In some embodiments, a reaction can be equilibrium driven until saturated, therefore, a free concentration of monomers can determine a detergent concentration. In some embodiments, SDS binding can be cooperative (i.e., a binding of one molecule of SDS increases a probability that another molecule of SDS will bind to that protein) and alters most proteins into rigid rods whose length can be proportional to molecular weight. In some embodiments, non-denaturing detergents such as Triton X-100 have rigid and bulky nonpolar heads that do not penetrate into water-soluble proteins; consequently, they generally do not disrupt native interactions and structures of water-soluble proteins and do not have cooperative binding properties. In some embodiments, a main effect of non-denaturing detergents can be to associate with hydrophobic parts of membrane proteins, thereby conferring miscibility to them. In some embodiments, a system for generating an isolated organ or portion thereof or tissue can be controlled by a computer-readable storage medium in combination with a programmable processor (e.g., a computer-readable storage medium as used herein has instructions stored thereon for causing a programmable processor to perform particular steps). In some embodiments, for example, such a storage medium, in combination with a programmable processor, can receive and process information from one or more sensors. In some embodiments, such a storage medium in conjunction with a programmable processor also can transmit information and instructions back to a bioreactor and/or an isolated organ or tissue. In some embodiments, an isolated organ or tissue undergoing recellularization can be monitored for biological activity. In some embodiments, biological activity can be that of an isolated organ or portion thereof or tissue itself such as for cardiac tissue, electrical activity, mechanical activity, mechanical pressure, contractility, and/or wall stress of an isolated organ or tissue. In some embodiments, a biological activity of cells attached or engrafted on to an isolated organ or portion thereof or tissue can be monitored, for example, for ion transport/exchange activity, cell division, and/or cell viability. In some embodiments, it can be useful to simulate an active load on an isolated organ or portion thereof during recellularization. In some embodiments, a computer-readable storage medium, in combination with a programmable processor, can be used to coordinate components necessary to monitor and maintain an active load on an isolated organ or tissue. In some embodiments, a weight of an isolated organ or portion thereof or tissue can be entered into a computer-readable storage medium as described herein, which, in combination with a programmable processor, can calculate exposure times and perfusion pressures for that particular organ or tissue. In some embodiments, such a storage medium can record preload and afterload (the pressure before and after perfusion, respectively) and a rate of flow. In some embodiments, for example, a computer-readable storage medium in combination with a programmable processor can adjust a perfusion pressure, a direction of perfusion, and/or a type of perfusion solution via one or more pumps and/or valve controls. In some embodiments, perfusion decellularization of an isolated organ or portion thereof can be from about 5%, 10%, 20%, 30%, 40%, 50%, 60%, 70%, 80%, 90%, 95%, or up to about 100% more effective as compared to a non-perfusion based decellularization system. In some embodiments, decellularization of an isolated organ or portion thereof can be determined using various means. In some embodiments, decellularization can be determined by histological examination. In some embodiments, histological examination can demonstrate a lack or reduction of cellular material, nuclei, and combinations thereof within an at least partially decellularized isolated organ or portion thereof with preservation of an overall structure such as lobules and central veins. In some embodiments, decellularization can be determined by immunohistochemical staining. In some embodiments, immunohistochemical staining can demonstrate paucity of cellular factors such as galactosyl-alpha (1,3) galactose (alpha-Gal) following perfusion decellularization. In some embodiments, decellularization can be determined using DNA quantification. In some embodiments, DNA quantification can comprise assays such as PicoGreen, qPCR, and Quant-IT. In some embodiments, DNA quantification assays can determine an amount of a reduction of DNA in an isolated organ or portion thereof. In some embodiments, decellularization can be determined by any of the methods of: histology with H&E staining, immunofluorescence for cellular structures, Immunohistochemical staining for cell membrane associated proteins, nuclear staining such as Dapi, or any combination thereof. In some embodiments, a perfusion-based decellularized isolated organ or portion thereof preserves a native scaffold containing an appropriate microenvironment required for an introduction of organ-specific cells, along with an intact vascular network to reconnect to a subject's blood supply and an outer capsule capable of maintaining physiologic pressures. In some embodiments, these components can be important for a later use of perfusion recellularization, which also uses perfusion to repopulate vascular and organ-specific regenerative cells onto an at least partially decellularized isolated organ or portion thereof, where they can migrate to an appropriate microenvironment (via a relevant signaling protein markers that remain within a perfusion decellularized scaffold) as an at least partially decellularized isolated organ or portion thereof grows and matures in a bioreactor under normal physiologic conditions. In some embodiments, a resulting at least partially recellularized isolated organ or portion thereof can then be transplanted utilizing a technique comparable to current organ transplantation. In some embodiments, scaffolds created by perfusion decellularization can be capable of receiving and incorporating a variety of cells. In some embodiments, a decellularization process can generate particulate. In some embodiments, particulate can refer to residual comments from a decellularization. In some embodiments, particulate can be formed by an insoluble interaction between native proteins and detergents. In some embodiments, particulate can be immiscible in aqueous solution. In some embodiments, a presentation of a non-soluble white particulate can be seen during decellularization of whole organs with SDS or other detergents. In some embodiments, particulate forms and then becomes trapped in an organ during decellularization, which can negatively affect a use of a decellularized matrix for acellular products or affect use of a decellularized isolated organ portion thereof or matrix thereof for recellularization. In some embodiments, a method can reduce particulate formation by a use of saline solutions, non-fasting mammals, and a combination thereof. In some embodiments, reducing particulate formation can decrease cytotoxicity of a decellularized matrix to introduced cells, for instance during recellularization. In some embodiments, reducing particulate can be performed by using solutions, controlling a mammal's eating habits, or their combination.

TABLE 1
Detergents that can be utilized in cellular disruption media

| | | | Agg.# | | | | | | |
|---|---|---|---|---|---|---|---|---|---|
| | | | (No. of | | | | | | |
| | | | molecules | MW | | CMC | | Cloud | |
| | | | per | mono | | mM | | point | |
| Detergent | Chemical name | Type | micelle) | (micelle) | | (% w/v) | | ° C. | Dialyzable |
| Triton X-100 | Polyethylene glycol p- | Nonionic | 140 | 647 | (90K) | 0.24 | (0.0155) | 64 | No |
| | (1,1,3,3-tetramethylbutyl)- | | | | | | | | |
| | phenyl ether | | | | | | | | |
| Triton X-114 | 1,1,3,3-Tetramethylbutyl)phenyl- | Nonionic | — | 537 | (—) | 0.21 | (0.0113) | 23 | No |
| | polyethylene glycol | | | | | | | | |
| NP-40 | 4-Nonylphenol, branched, | Nonionic | 149 | 617 | (90K) | 0.29 | (0.0179) | 80 | No |
| | ethoxylated | | | | | | | | |
| Brij-35 | Polyoxyethylene lauryl ether | Nonionic | 40 | 1225 | (49K) | 0.09 | (0.0110) | >100 | No |
| Brij-58 | Polyethylene glycol hexadecyl | Nonionic | 70 | 1120 | (82K) | 0.08 | (0.0086) | >100 | No |
| | ether | | | | | | | | |
| Tween 20 | Polyethylene glycol sorbitan | Nonionic | — | 1228 | (—) | 0.06 | (0.0074) | 95 | No |
| | monolaurate | | | | | | | | |
| Tween 80 | Polyethylene glycol sorbitan | Nonionic | 60 | 1310 | (76K) | 0.01 | (0.0016) | | No |
| | monooleate | | | | | | | | |
| Octyl glucoside | n-octyl-β-D-glucoside | Nonionic | 27 | 292 | (8K) | 23-24 | (~0.70) | >100 | Yes |
| Octyl | Octyl | Nonionic | — | 308 | (—) | 9 | (0.2772) | >100 | Yes |
| thioglucoside | β-D-1-thioglucopyranoside | | | | | | | | |
| Sodium dodecyl | Dodecyl sodium sulfate | Anionic | 62 | 288 | (18K) | 6-8 | (0.17-0.23) | >100 | No |
| sulfate | | | | | | | | | |
| CHAPS | 3-[(3- | Zwitterionic | 10 | 615 | (6K) | 8-10 | (0.5-0.6) | >100 | Yes |
| | Cholamidopropyl)dimethyl- | | | | | | | | |
| | ammonio]-1-propanesulfonate | | | | | | | | |
| | hydrate | | | | | | | | |
| CHAPSO | 3-([3- | Zwitterionic | 11 | 631 | (7K) | 8-10 | (~0.505) | 90 | Yes |
| | Cholamidopropyl]dimethyl- | | | | | | | | |
| | ammonio)-2-hydroxy-1- | | | | | | | | |
| | propanesulfonate | | | | | | | | |

### Immersion Decellularization

Also disclosed herein in some embodiments, are methods of immersion-based decellularization of an isolated organ or portion thereof. In some embodiments, whole organs or portions thereof can be decellularized by removing an entire cellular and tissue content from an organ. In some embodiments, decellularization can comprise a series of sequential extractions. In some embodiments, a first step can involve removal of cellular debris and solubilization of a cell membrane. In some embodiments, this can be followed by solubilization of a nuclear cytoplasmic component and a nuclear component. In some embodiments, an isolated organ can be decellularized by removing a cell membrane and cellular debris surrounding an isolated organ using gentle mechanical disruption methods. In some embodiments, a gentle mechanical disruption method can disrupt a cellular membrane. In some embodiments, a process of decellularization can avoid damage or disturbance of a biostructure's complex infra-structure. In some embodiments, gentle mechanical disruption methods can include scraping a surface of an isolated organ or portion thereof, agitating an isolated organ or portion thereof, or stirring an isolated organ or portion thereof in a suitable volume of fluid, e.g., distilled water. In some embodiments, a gentle mechanical disruption method can include magnetically stirring (e.g., using a magnetic stir bar and a magnetic plate) an isolated organ or portion thereof in a suitable volume of distilled water until a cell membrane can be disrupted and a cellular debris has been removed from an isolated organ or portion thereof. In some embodiments, after a cell membrane has been removed, a nuclear or cytoplasmic biostructure component can be removed. In some embodiments, this can be performed by solubilizing cellular or nuclear components without disrupting an infra-structure. In some embodiments, to solubilize a nuclear component, non-ionic detergents or surfactants can be used. In some embodiments, examples of nonionic detergents or surfactants include, but are not limited to, a Triton series surfactant, available from Rohm and Haas of Philadelphia, Pa., which includes Triton X-100, Triton N-101, Triton X-114, Triton X-405, Triton X-705, and Triton DF-16, available commercially from many vendors; a Tween series surfactant, such as monolaurate (Tween 20), monopalmitate (Tween 40), monooleate (Tween 80), and polyoxethylene-23-lauryl ether (Brij. 35), polyoxyethylene ether W-1 (Polyox), sodium cholate, deoxycholates, CHAPS, saponin, n-Decyl β-D-glucopuranoside, n-heptyl β-D glucopyranoside, n-Octylα-D-glucopyranoside, Nonidet β-40, or any combination thereof.

### Physical Treatments

Also disclosed herein in some embodiments, are methods of at least partial decellularization by means of physical treatment of an isolated organ or portion thereof. In some embodiments, physical treatment can be used to lyse, kill, and remove cells from an ECM or portion thereof. In some embodiments, physical treatment can utilize temperature, force, pressure, and electrical disruption. In some embodiments, temperature methods can be used in a rapid freeze-thaw mechanism. In some embodiments, for example, by freezing a tissue, microscopic ice crystals can form around a plasma membrane and a cell can be lysed. In some embodiments, after lysing one or more cells, a tissue can be further exposed to liquidized chemicals that can degrade and wash out any residual or undesirable components. In some embodiments, temperature methods can conserve a physical structure of an ECM scaffold. In some embodiments, an isolated organ or portion thereof, and a tissue can be decellularized at a suitable temperature. In some embodiments, a suitable temperature can be from about 4° C., 8° C., 10° C., 12° C., 14° C., 16° C., 18° C., 20° C., 22° C., 24° C., 26° C., 28° C., 30° C., 32° C., 34° C., 36° C., 38° C., 40° C., 45° C., 50° C., 55° C., 60° C., or up to about 70° C. In some embodiments, a physical treatment can also include a use of pressure. In some embodiments, pressure decellularization can involve a controlled use of hydrostatic pressure applied to a tissue, isolated organ, or portion thereof. In some embodiments, pressure decellularization can be performed at high temperatures to avoid unmonitored ice crystal formation. In some embodiments, electrical disruption of an isolated organ or portion thereof can be performed. In some embodiments, electrical disruption can be done to lyse cells housed in a tissue or isolated organ or portion thereof. In some embodiments, by exposing a tissue, isolated organ, or portion thereof to electrical pulses, micropores can be formed at a plasma membrane. In some embodiments, one or more cells can die after their homeostatic electrical balance can be ruined through such applied stimulus. In some embodiments, this electrical process can be documented as Non-thermal irreversible electroporation (NTIRE). In some embodiments, sonication can also be used for decellularization or to enhance perfusion decellularization.

### Chemical and Enzymatic Treatments

Also disclosed herein in some embodiments, are methods of chemical treatment of an isolated organ or portion thereof to achieve at least partial decellularization. In some embodiments, chemicals and/or salts thereof for use in a chemical treatment can be selected for decellularization depending on: thickness, extracellular matrix composition, intended use of a tissue or isolated organ, or any combination thereof. In some embodiments, for example, enzymes may not be used on a collagenous tissue because they can disrupt connective tissue fibers. In some embodiments, when collagen may not be present in a high concentration or needed in a tissue, enzymes can be a viable option for decellularization. In some embodiments, a chemical or salt thereof which can be used to kill and remove cells can be but are not limited to acids, alkaline treatments, ionic detergents, non-ionic detergents, zwitterionic detergents, or any combination thereof. In some embodiments, one or more chemicals can comprise a cellular disruption media. In some embodiments, a cellular disruption media can comprise at least one detergent such as Sodium dodecyl sulfate (SDS), polyethyleneglycol (PEG), or Triton X. In some embodiments, detergents can act effectively to lyse a cell membrane and expose content to further degradation. In some embodiments, for example, after SDS lyses a cellular membrane, endonucleases and/or exonucleases can degrade a genetic content, while other components of a cell can be solubilized and washed out of a matrix. In some embodiments, a detergent can be mixed with an alkaline and/or acid treatment due to their ability to degrade nucleic acids and solubilize cytoplasmic inclusions. In some embodiments, one or more cellular disruption media can be used to decellularize an isolated organ or tissue. In some embodiments, a cellular disruption medium can comprise at least one detergent such as SDS, PEG, or Triton X. In some embodiments, a cellular disruption medium can comprise water such that a media can be osmotically incompatible with a cell. In some embodiments, alternatively, a cellular disruption medium can comprise a buffer (e.g., PBS) for osmotic compatibility with a cell. In some embodiments, cellular disruption media also can include enzymes such as, without limitation, one or more collagenases, one or more dispases, one or more DNases, one or more proteases, and any combination thereof. In some embodiments, cellular disruption media also or alternatively can include inhibitors of one or more enzymes (e.g., protease inhibitors, nuclease inhibitors, and/or collagenase inhibitors). In some embodiments, a cellular disruption media can include water such that a media can be osmotically incompatible with a cell. In some embodiments, alternatively, a cellular disruption media can include a buffer (e.g., PBS) for osmotic compatibility with a cell. In some embodiments, cellular disruption media also can include enzymes such as, without limitation, one or more collagenases, one or more dispases, one or more DNases, or a protease such as trypsin. In some embodiments, cellular disruption media also or alternatively can include inhibitors of one or more enzymes (e.g., protease inhibitors, nuclease inhibitors, and/or collagenase inhibitors). In some embodiments, a non-ionic detergent such as Triton X-100 can be utilized. In some embodiments, Triton X-100 can disrupt an interaction between lipids, or between lipids and proteins. In some embodiments, Triton X-100 may not disrupt protein-protein interactions, which can be beneficial to keeping an ECM intact. In some embodiments, EDTA can be utilized. In some embodiments, EDTA can be a chelating agent that binds calcium, which can be a component for proteins to interact with one another. In some embodiments, by making calcium unavailable, EDTA can prevent integral proteins between cells from binding to one another. In some embodiments, EDTA can be used with trypsin, an enzyme that acts as a protease to cleave an already existing bond between integral proteins of neighboring cells within a tissue. In some embodiments, a detergent can be administered from about 10 min, 30 min, 60 min, 1 hr., 2 hrs., 3 hrs., 4 hrs., 5 hrs., 6 hrs., 7 hrs., 8 hrs., 9 hrs., 10 hrs., 11 hrs., 12 hrs., 13 hrs., 14 hrs., 15 hrs., 16 hrs., 17 hrs., 18 hrs., 19 hrs., 20 hrs., 21 hrs., 22 hrs., 23 hrs., 24 hrs., 25 hrs., 26 hrs., 27 hrs., 28 hrs., 29 hrs., 30 hrs., 31 hrs., 32 hrs., 33 hrs., 34 hrs., 35 hrs., 36 hrs., 37 hrs., 38 hrs., 39 hrs., 40 hrs., 41 hrs., 42 hrs., 43 hrs., 44 hrs., 45 hrs., 46 hrs., 47 hrs., 48 hrs., 49 hrs., 50 hrs., 51 hrs., 52 hrs., 53 hrs., 54 hrs., 55 hrs., 56 hrs., 57 hrs., 58 hrs., 59 hrs., 60 hrs., 70 hrs., 80 hrs., 90 hrs., or up to about 100 hrs. In some embodiments, depending upon a size and/or weight of an isolated organ or portion thereof a chemical treatment such as a detergent can be contacted with an isolated organ or portion thereof from about 2 hours, 3 hours, 4 hours, 5 hours, 6 hours, 7 hours, 8 hours, 9 hours, 10 hours, 11 hours, 12 hours, 13 hours, 14 hours, 15 hours, to about 20 hours per gram of solid isolated organ or tissue with cellular disruption medium. In some embodiments, including washes, an isolated organ can be perfused for up to about 12 hrs., 13 hrs., 14 hrs., 15 hrs., 16 hrs., 17 hrs., 18 hrs., 19 hrs., 20 hrs., 21 hrs., 22 hrs., 23 hrs., 24 hrs., 25 hrs., 26 hrs., 27 hrs., 28 hrs., 29 hrs., 30 hrs., 31 hrs., 32 hrs., 33 hrs., 34 hrs., 35 hrs., 36 hrs., 37 hrs., 38 hrs., 39 hrs., 40 hrs., 41 hrs., 42 hrs., 43 hrs., 44 hrs., 45 hrs., 46 hrs., 47 hrs., 48 hrs., 49 hrs., 50 hrs., 51 hrs., 52 hrs., 53 hrs., 54 hrs., 55 hrs., 56 hrs., 57 hrs., 58 hrs., 59 hrs., 60 hrs., 70 hrs., 80 hrs., 90 hrs., or up to about 100 hrs. In some embodiments, an isolated organ or portion thereof can be perfused from about 12 hours to about 72 hours per gram of tissue. In some embodiments, perfusion can be adjusted to physiologic conditions including pulsatile flow, rate, pressure, and any combination thereof. In some embodiments, an isolated organ, portion thereof, or tissue can be contacted sequentially with at least two different cellular disruption media. In some embodiments, for example, a first cellular disruption medium can include an anionic detergent such as SDS and a second cellular disruption medium can include an ionic detergent such as Triton X. In some embodiments, following contacting, such as perfusion, with at least one cellular disruption medium, a cannulated isolated organ or tissue can be perfused, for example, with wash solutions or solutions containing one or more enzymes. In some embodiments, alternating a direction of perfusion (e.g., antegrade and retrograde) can help to effectively decellularize an isolated organ, portion thereof, or tissue. In some embodiments, decellularization can decellularize an isolated organ or portion thereof from an inner portion toward an outer portion, resulting in very little damage to an ECM. In some embodiments, a sequential method of decellularization can comprise contacting an isolated organ or portion thereof with a cellular disruption media, such as an SDS detergent, followed by a washing step, followed by an addition of one or more chemicals, followed by contacting with a detergent, and ending with at least one wash step. In some embodiments, a sequential method of decellularization can comprise at least 1, 2, 3, 4, 5, 6, 7, 8, 9, 10, 11, 12, 13, 14, or up to 15 contacting steps with any media or solution disclosed herein. In some embodiments, a buffer disclosed herein can be at a concentration from about 0.1%, 0.5%, 1%, 1.5%, 2%, 2.5%, 3%, 3.5%, 4%, 4.5%, 5%, 10%, 20%, 30%, 40%, 50%, 60%, 70%, 80%, 90%, 95%, or up to about 100%. In some embodiments, an isolated organ, a tissue, or a portion thereof (e.g., liver, lung, kidney, heart, bladder, pancreas, spleen, uterus, or a portion thereof) can be pretreated or flushed with a flushing solution prior to decellularization. In some embodiments, pretreating or flushing an isolated organ, tissue, or a portion thereof with a solution can be used to remove blood and/or reduce blood clot formation. In some embodiments, an isolated organ, a tissue, or a portion thereof can be flushed once, twice, three times, four times, five times, or longer. In some embodiments, flushing occurs over a period of time. In some embodiments, flushing an isolated organ, tissue, or portion thereof can range from a few minutes to days, a few minutes to a few hours, or for a few hours (e.g., for about 5 minutes, 10 minutes, 15 minutes, 20 minutes, 30 minutes, 45 minutes, 1 hour, 2 hours, 3 hours, 6 hours, 10 hours, 24 hours, 48 hours, or longer). In some embodiments, a flushing solution can be a hypertonic solution. In some embodiments, a hypertonic solution can refer to a solution that has a greater concentration of solutes than a concentration of solutes inside of a cell. In some embodiments, a hypertonic solution includes sodium chloride and a second sodium source, such as sodium acetate. In some embodiments, a hypertonic solution includes a source of sodium and a source of chloride. In some embodiments, a ratio of sodium to chloride can be between about 1:0.7 and 1:1. In some embodiments, hypertonic solutions can comprise: NaCl, KCl, NaHCO₃, Na₂SO₄, Na₂HPO₄, MgCl₂, Na acetate, Na lactate, saline, 4× saline, 5× saline, or any combination thereof. In some embodiments, a hypertonic solution can be saline. In some embodiments, a hypertonic solution can be 5× saline. In some embodiments, a hypertonic solution can be about, not less than about, or at most about 0.10% NaCl, 0.2% NaCl, 0.5% NaCl, 0.7% NaCl, 0.8% NaCl, 0.9% NaCl, 10% NaCl, 1.5% NaCl, 2% NaCl 2.5% NaCl, 3% NaCl, 3.5% NaCl, 4% NaCl, 4.5% NaCl, 5% NaCl, 5.5% NaCl, 6% NaCl, 6.5% NaCl, 7% NaCl, 8% NaCl, 9% NaCl, 10% NaCl, 12% NaCl, 15% NaCl, 20% NaCl, 23% NaCl, or 25% NaCl. In some embodiments, a hypertonic solution can include other ingredients, such as KHCO₃, K acetate, K lactate, MgSO₄, or K₂HPO₄. In some embodiments, a saline solution can have a formulation of 10× which can be diluted with water to attain various hypertonic solutions. In some embodiments, a hypertonic solution can range from about 1.1× to about 10× buffered solution. In some embodiments, a buffered solution can be a saline solution. In some embodiments, an isolated organ, a tissue, or any portion thereof can be washed with a hypertonic solution, a hypotonic solution, or both. In some embodiments, an isolated organ, a tissue, or a portion thereof can be disinfected before and/or after decellularization. In some embodiments, disinfection can be performed before decellularization. In some embodiments, disinfection can be performed after decellularization. In some embodiments, disinfection can be performed after an isolated organ, tissue, or portion thereof can be flushed with a flushing solution. In some embodiments, disinfection can be performed without flushing an isolated organ, tissue, or portion thereof with a flushing solution. In some embodiments, disinfection can be performed before an isolated organ, tissue, or portion thereof can be washed with a washing solution. In some embodiments, disinfecting an isolated organ, tissue, or portion thereof can be performed, for example, by placing an isolated organ, tissue, or portion thereof in a disinfection bath with a disinfecting solution. In some embodiments, disinfection can also occur by perfusing or immersing an isolated organ, tissue, or portion thereof with a disinfection solution. In some embodiments, disinfection occurs by submersing an isolated tissue, organ, or portion thereof in a disinfection solution. In some embodiments, a disinfection solution can include an acid, peracid, hydrogen peroxide, peroxide, acetic acid, peracetic acid, and peroxyacetic acid. In some embodiments, a disinfecting solution can comprise one or more of a peracid, hydrogen peroxide, acetic acid, peracetic acid (PAA), saline, SDS, or sodium hydroxide (NaOH). In some embodiments, a solution used for disinfection can comprise an acid (e.g., a peracid), hydrogen peroxide, a chemical compound comprising hydrogen peroxide, a chemical compound comprising a peracid, hydrogen peroxide covalently linked to an organic moiety, saline, and/or a sodium containing solution. In some embodiments, a disinfection solution can comprise saline. In some embodiments, a disinfection solution can comprise sodium. In some embodiments, a disinfection solution can comprise NaCl (e.g., 0.1% NaCl, 0.2% NaCl, 0.5% NaCl, 0.7% NaCl, 0.8% NaCl, 0.9% NaCl, 1% NaCl, 1.5% NaCl, 2% NaCl 2.5% NaCl, 3% NaCl, 3.5% NaCl, 4% NaCl, 4.5% NaCl, 5% NaCl, 5.5% NaCl, 6% NaCl, 6.5% NaCl, 7% NaCl, 8% NaCl, 9% NaCl, 10% NaCl, 12% NaCl, 15% NaCl, 20% NaCl, 23% NaCl, or 25% NaCl). In some embodiments, a disinfection solution can comprise saline and an acid. In some embodiments, a disinfection solution can comprise saline and peracid (e.g., peracetic acid). In some embodiments, a disinfection solution can comprise 0.9% NaCl and 600 ppm peracetic acid. In some embodiments, saline can be 1× saline, 2× saline, 5× saline, 7× saline, 10× saline, 12× saline, 15× saline. In some embodiments, a disinfection solution can comprise saline and a peracid (e.g., peracetic acid). In some embodiments, saline can be 1× saline. In some embodiments, an acid or peracid in a disinfection solution can range from about 25 parts per million (ppm) to about 4000 ppm. In some embodiments, an acid or peracid in a disinfection solution can range from about 500 ppm to about 700 ppm (500 ppm-700 ppm), 600 ppm-650 ppm, 250 ppm-700 ppm, 250 ppm-800 ppm, 550 ppm-1000 ppm, 600 ppm-700 ppm, 550 ppm-2000 ppm, 550 ppm-3000 ppm, 550 ppm-4000 ppm, 1000 ppm-2000 ppm, 2000 ppm-3000 ppm, or 3000 ppm-4000 ppm. In some embodiments, an acid or peracid can be about, at least about, or at most about 10 ppm, 25 ppm, 50 ppm, 75 ppm, 90 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 900 ppm, 1000 ppm, 1200 ppm, 1400 ppm, 1500 ppm, 1700 ppm, 2000 ppm, 2200 ppm, 2500 ppm, 2750 ppm, 3000 ppm, 3200 ppm, 3500 ppm, 3750 ppm, or 4000 ppm. In some embodiments, an acid or peracid (e.g., peracetic acid) can be about 600 ppm. In some embodiments, an acid or peracid (e.g., peracetic acid) can be about 50 ppm. In some embodiments, a disinfection solution can have a pH of about 4 to about 10 (e.g., pH of 6-7, 5-8, 5-10, 6-8, and 5.5-9). In some embodiments, a disinfecting solution has a pH of between about 5.00 to about 7.50, between about 6.00 to about 8.00, between about 6.10 to about 7.00, between about 4.50 to about 9.00, or between about 6.00 to about 6.50. In some embodiments, a disinfecting solution has a pH of about, at least about, or at most about 4.00, 4.50, 5.00, 5.50, 5.80, 5.90, 6.00, 6.05, 6.10, 6.11, 6.12, 6.13, 6.14, 6.15, 6.16, 6.17, 6.18, 6.19, 6.20, 6.30, 6.40, 6.41, 6.42, 6.43, 6.44, 6.45, 6.50, 6.60, 6.90, 7.00, 7.50, 8.00, 8.50, 9.0, 9.5, 10, 10.5, 11, 11.5, 12, 12.5, 13, 13.5, or 14. In some embodiments, disinfection of an isolated organ, a tissue, or a portion thereof can range from a few minutes, to days, to weeks, to months, or longer. In some embodiments, disinfection can be performed for about, at least about, or at most about 5 minutes, 10 min., 20 min., 30 min., 45 min., 1 hr., 1.5 hrs., 2 hrs., 2.5 hrs., 3 hrs., 3.5 hrs., 4 hrs., 4.5 hrs., 5 hrs., 5.5 hrs., 6 hrs., 6.5 hrs., 7 hrs., 7.5 hrs., 8 hrs., 8.5 hrs., 9 hrs., 9.5 hrs., 10 hrs., 12 hrs., 15 hrs., 18 hrs., 20 hrs., 22 hrs., 24 hrs., 27 hrs., 30 hrs., 34 hrs., 40 hrs., 44 hrs., 48 hrs., or longer. In some embodiments, disinfection can be performed for about, at least about, or at most about a day, two days, three days, four days, five days, six days, seven days, fourteen days, thirty days, or longer. In some embodiments, disinfection can be performed for about, at least about, or at most about a week, two weeks, three weeks, four weeks, five weeks, seven weeks, eight weeks, a month, two months, three months, four months, five months, six months, a year, or longer.

In some embodiments, an isolated organ, tissue, or portion thereof can be decellularized using a decellularization solution comprising an acid (e.g. acetic acid) or a radical generating compound. A radical generating compound can be a compound that can be capable of decomposing into a radical under mild conditions. A radical generating compound can include a compound of formula R¹—O—O—R², wherein R¹ and R² can independently be H, C₁-C₆ alkyl, substituted C₁-C₆ alkyl, aryl, substituted aryl, benzyl, substituted benzyl, C(═O)A¹, C(═O)OA², wherein A₁ and A₂ can independently be H, C₁-C₆ alkyl or substituted C₁-C₆ alkyl. In some embodiments, a radical generating compound can be a compound of formula R¹—O—O—R², wherein R¹ can be H and R² can be C(═O)CH₃. In some cases, a radical generating compound can be a peracid as described herein.

In some embodiments, decellularization of an isolated organ, tissue, or portion thereof can be performed after disinfection. In some embodiments, decellularization of an isolated organ, tissue, or portion thereof can be performed before disinfection. In some embodiments, decellularization of an isolated organ, tissue, or portion thereof can be performed after flushing. In some embodiments, decellularization of an isolated organ, tissue, or portion thereof can be performed before flushing. In some embodiments, decellularization of an isolated organ, tissue, or portion thereof can be performed after flushing and disinfection. In some embodiments, decellularization of an isolated organ, tissue, or portion thereof can be performed before flushing and disinfection. In some embodiments, a solution used for decellularization can comprise a detergent (e.g., sodium dodecyl sulfate (SDS)). In some embodiments, a solution used for decellularization can comprise a detergent and an acid such as a peracid. In some embodiments, a solution used for decellularization can comprise an acid, a peracid, hydrogen peroxide, a chemical compound comprising hydrogen peroxide, a chemical compound comprising a peracid, hydrogen peroxide covalently linked to an organic moiety, saline, and/or a sodium containing solution. In some embodiments, a detergent (e.g., SDS) can be present at an amount of about 0.1%, 0.2%, 0.3%, 0.5%, 0.6%, 0.8%, 0.9%, 1%, 5%, 10%, 15%, 17%, 20%, 25%, 30%, 35%, 40%, 50%, 60%, 65%, 70%, 80%, 90%, or greater. In some embodiments, a detergent can be present at 0.6%. In some embodiments, a detergent can be present at 0.9%. In some embodiments, a detergent can be commercially available. In some embodiments, a solution used for decellularization can comprise a detergent and a peracid (e.g., SDS and peracetic acid (PAA)). In some embodiments, a decellularization solution can have a pH of about 4 to about 10 (e.g., pH of 6-7, 5-8, 5-10, 6-8, and 5.5-9). In some embodiments, a decellularization solution has a pH of between about 5.00 to about 7.50, between about 6.00 to about 8.00, between about 6.10 to about 7.00, between about 4.50 to about 9.00, or between about 6.00 to about 6.50. In some embodiments, a decellularization solution has a pH of about, at least about, or at most about 4.00, 4.50, 5.00, 5.50, 5.80, 5.90, 6.00, 6.05, 6.10, 6.11, 6.12, 6.13, 6.14, 6.15, 6.16, 6.17, 6.18, 6.19, 6.20, 6.30, 6.40, 6.41, 6.42, 6.43, 6.44, 6.45, 6.50, 6.60, 6.90, 7.00, 7.50, 8.00, 8.50, 9.0, 9.5, 10, 10.5, 11, 11.5, 12, 12.5, 13, 13.5, or 14. In some embodiments, decellularization of an isolated organ, a tissue, or a portion thereof can range from a few minutes, to days, to weeks, to months, or longer. In some embodiments, decellularization can be performed for about, at least about, or at most about 5 minutes, 10 min., 20 min., 30 min., 45 min., 1 hr., 1.5 hrs., 2 hrs., 2.5 hrs., 3 hrs., 3.5 hrs., 4 hrs., 4.5 hrs., 5 hrs., 5.5 hrs., 6 hrs., 6.5 hrs., 7 hrs., 7.5 hrs., 8 hrs., 8.5 hrs., 9 hrs., 9.5 hrs., 10 hrs., 12 hrs., 15 hrs., 18 hrs., 20 hrs., 22 hrs., 24 hrs., 27 hrs., 30 hrs., 34 hrs., 40 hrs., 44 hrs., 48 hrs., or longer. In some embodiments, decellularization can be performed for about, at least about, or at most about a day, two days, three days, four days, five days, six days, seven days, fourteen days, thirty days, or longer. In some embodiments, disinfection can be performed for about, at least about, or at most about a week, two weeks, three weeks, four weeks, five weeks, seven weeks, eight weeks, a month, two months, three months, four months, five months, six months, a year, or longer. In some embodiments, a decellularization can comprise: at least one wash step, at least one flushing step, at least one disinfection step, at least one decellularization step. In some embodiments, a peracid solution can be a peroxide solution. In some embodiments, examples of peracid include, but are not limited to, peroxyacetic acid, peroxyoctanoic acid, a sulfoperoxycarboxylic acid, peroxysulfonated oleic acid, peroxyformic acid, peroxyoxalic acid, peroxypropanoic acid, peroxybutanoic acid, peroxypentanoic acid, peroxyhexanoic acid, peroxyadipic acid, perlactic acid, peroxycitric, peroxybenzoic acid, and any combination thereof. In some embodiments, a peracid can be a mixture of hydrogen peroxide and acetic acid. In some embodiments, a peracid can be peracetic acid (PAA). In some embodiments, a peracid (e.g., peracetic acid) can be present at a range of about 0.1% to about 1.0%, of about 0.1% to about 15%, of about 0.1% to about 5.0%, of about 0.1% to about 10%, or of about 0.1% to about 0.5%. In some embodiments, a solution containing a peracid (e.g., peracetic acid) can comprise about 0.1% peracid. In some embodiments, a peracid concentration in a solution (e.g., in a decellularization solution or disinfection solution or another solution used during a decellularization process) can be about, at least about, or at most about 0.1%, 0.3%, 0.5%, 0.8%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 18%, 20%, 23%, 25%, or more. In some embodiments, an acid or peracid in a solution (e.g., in a decellularization solution or disinfection solution or another solution used during a decellularization process) can range from about 25 parts per million (ppm) to about 4000 ppm. In some embodiments, an acid or peracid in a solution (e.g., in a decellularization solution or disinfection solution or another solution used during a decellularization process) can range from about 500 ppm to about 700 ppm (500 ppm-700 ppm), 600 ppm-650 ppm, 250 ppm-700 ppm, 250 ppm-800 ppm, 550 ppm-1000 ppm, 600 ppm-700 ppm, 550 ppm-2000 ppm, 550 ppm-3000 ppm, 550 ppm-4000 ppm, 1000 ppm-2000 ppm, 2000 ppm-3000 ppm, or 3000 ppm-4000 ppm. In some embodiments, an acid or peracid in a solution (e.g., in a decellularization solution or disinfection solution or another solution used during a decellularization process) can be about, at least about, or at most about 10 ppm, 25 ppm, 50 ppm, 75 ppm, 90 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 900 ppm, 1000 ppm, 1200 ppm, 1400 ppm, 1500 ppm, 1700 ppm, 2000 ppm, 2200 ppm, 2500 ppm, 2750 ppm, 3000 ppm, 3200 ppm, 3500 ppm, 3750 ppm, or 4000 ppm. In some embodiments, an acid or peracid (e.g., peracetic acid) can be about 600 ppm. In some embodiments, an acid or peracid (e.g., peracetic acid) can be about 50 ppm. In some embodiments, an addition of an acid or peracid to a solution can increase a flow rate. In some embodiments, an increase in flow rate can be retained even in subsequent steps without an acid or peracid. In some embodiments, an addition of a peracid to a solution during perfusion decellularization can increase a flow rate and a flow rate can be retained (or does not substantially decrease) during subsequent perfusions without a peracid. In some embodiments, a flow rate can be adjusted at a predetermined pressure. In some embodiments, a predetermined pressure can be about, at least about, or at most about 1 mmHg, 2 mmHg, 3 mmHg, 4 mmHg, 5 mmHg, 6 mmHg, 7 mmHg, 8 mmHg, 9 mmHg 10 mmHg, 11 mmHg, 12 mmHg, 13 mmHg, 14 mmHg, 15 mmHg, 16 mmHg, 17 mmHg, 18 mmHg, 19 mmHg, 20 mmHg, 21 mmHg, 22 mmHg, 23 mmHg, 24 mmHg, 25 mmHg, 26 mmHg, 27 mmHg, 28 mmHg, 29 mmHg 30 mmHg, 35 mmHg, 40 mmHg, 45 mmHg, 50 mmHg, 55 mmHg, 60 mmHg, 65 mmHg, 70 mmHg, 75 mmHg, 80 mmHg, 90 mmHg, 100 mmHg, 150 mmHg, 175 mmHg, 200 mmHg, 250 mmHg, 300 mmHg, 350 mmHg, 400 mmHg, 450 mmHg, 500 mmHg, 550 mmHg, 600 mmHg, 700 mmHg, 800 mmHg, 900 mmHg, 1000 mmHg, or more. In some embodiments, a predetermined pressure can be between about 5 mmHg to about 20 mmHg, between about 10 mmHg to about 30 mmHg, between about 5 mmHg to about 50 mmHg, or between about 8 mmHg to about 100 mmHg. In some embodiments, a flow rate can be increased by about, at least about, or at most about 50 ml/min, 100 ml/min, 150 ml/min, 200 ml/min, 250 ml/min, 300 ml/min, 350 ml/min, 400 ml/min, 450 ml/min, 500 ml/min, 550 ml/min, 600 ml/min, 650 ml/min, 700 ml/min, 750 ml/min, 800 ml/min, 900 ml/min, 1000 ml/min, 1500 ml/min, 2000 ml/min, or higher. In some embodiments, a flow rate can be increased above a physiological rate. In some embodiments, a flow rate can be increased above a physiological rate by about 5%, 10%, 15%, 20%, 25%, 30%, 35%, 40%, 45%, 50%, 55%, 60%, 70%, 80%, 90%, 95% or higher. In some embodiments, a flow rate can be increased to about, at least about, or at most about 500 mL/min, 1000 mL/min, 1500 mL/min, 2000 mL/min, 2500 m/min, or higher. In some embodiments, a flow rate can be measured by chromatography such as by liquid chromatography (LC), gas chromatography (GC), or supercritical fluid chromatography (SFC). In some embodiments, a decellularization process can last about, can be at least about, or at most about 10 days, 8 days, 7 days, 5 days, 4 days, 3 days, 2 days, 1 day or less than 1 day. In some embodiments, a process can last about, be at least about, or at most about 1 hr., 2 hrs., 3 hrs., 4 hrs., 5 hrs., 6 hrs., 7 hrs., 10 hrs., 15 hrs., 18 hrs., 20 hrs., 24 hrs., 30 hrs., 35 hrs., 40 hrs., 45 hrs., 50 hrs., 60 hrs., 70 hrs., 80 hrs., 90 hrs., 100 hrs., or more than about 100 hrs. In some embodiments, a decellularization process includes for example, perfusing or immersing or submerging an isolated organ, tissue, or portion thereof with a detergent solution for about 1 to about 10, about 1 to about 20, about 1 to about 7, about 1 to about 15, about 1 to about 2, about 1 to about 5, or about 1 to about 6 phases or cycles. In some embodiments, a decellularization process includes for example, perfusing or immersing or submerging an isolated organ, tissue, or portion thereof with a detergent solution for a total of about, at least about, or at most about 1 hr., 2 hrs., 3 hrs., 4 hrs., 5 hrs., 6 hrs., 7 hrs., 8 hrs., 9 hrs., 10 hrs., 12 hrs., 15 hrs., 20 hrs., 25 hrs., 30 hrs., 35 hrs., 40 hrs., 45 hrs., 50 hrs., 60 hrs., 70 hrs., 80 hrs., 90 hrs., 100 hrs., or longer. In some embodiments, a decellularization process includes for example, perfusing or immersing or submerging an isolated organ, tissue, or portion thereof with a detergent solution for a total of about 20 hrs. to about 50 hrs., of about 10 hrs. to about 80 hrs., or of about 30 hrs. to about 50 hrs. In some embodiments, a decellularization process includes for example, perfusing or immersing or submerging an isolated organ, tissue, or portion thereof with a peracid (e.g., PAA) solution for about 1 to about 10, about 1 to about 20, about 1 to about 7, about 1 to about 15, about 1 to about 2, about 1 to about 5, about 1 to about 3, about 1 to about 2, or about 1 to about 6 phases or cycles. In some embodiments, a decellularization process includes for example, perfusing or immersing or submerging an isolated organ, tissue, or portion thereof with a peracid solution for a total of about, at least about, or at most about 1 hr., 2 hrs., 3 hrs., 4 hrs., 5 hrs., 6 hrs., 7 hrs., 8 hrs., 9 hrs., 10 hrs., 12 hrs., 15 hrs., 20 hrs., 25 hrs., 30 hrs., 35 hrs., 40 hrs., 45 hrs., 50 hrs., 60 hrs., 70 hrs., 80 hrs., 90 hrs., 100 hrs., or longer. In some embodiments, a decellularization process includes for example, perfusing or immersing or submerging an isolated organ, tissue, or portion thereof with a peracid solution for a total of about 2 hrs. to about 10 hrs., of about 30 hrs. to about 7 hrs., or of about 1 hr. to about 15 hrs. In some embodiments, a detergent solution and a peracid solution overlap. In some embodiments, a detergent solution can comprise a peracid solution. In some embodiments, a decellularization process includes washes with water. In some embodiments, a decellularization process can include: perfusing, immersing or submerging an isolated organ, tissue, or portion thereof with water for a total of about, at least about, or at most about 1 hr., 2 hrs., 3 hrs., 4 hrs., 5 hrs., 6 hrs., 7 hrs., 8 hrs., 9 hrs., 10 hrs., 12 hrs., 15 hrs., 20 hrs., 25 hrs., 30 hrs., 35 hrs., 40 hrs., 45 hrs., 50 hrs., 60 hrs., 70 hrs., 80 hrs., 90 hrs., 100 hrs., or longer. In some embodiments, a decellularization process can include: perfusing or immersing or submerging an isolated organ, tissue, or portion thereof with water for about 1 to about 10, about 1 to about 20, about 1 to about 7, about 1 to about 15, about 1 to about 2, about 1 to about 5, about 1 to about 3, about 1 to about 2, or about 1 to about 6 phases or cycles. In some embodiments, a wash with water can contain PBS (e.g., between about 5% PBS up to about 60% PBS). In some embodiments, water can be specific for an isolated organ, tissue, or portion thereof (e.g., hepatic water for liver decellularization). In some embodiments, a decellularized isolated organ, tissue, or portion thereof can comprise at most about 50% of native cells, 40% of native cells, 35% of native cells, 30% of native cells, 20% of native cells, 10% of native cells, 5% of native cells, 3% of native cells, 2% of native cells, 1% of native cells, or about 0% of native cells.

Also disclosed herein in some embodiments, are methods of decellularization of an isolated organ, a tissue, or a portion thereof which may not compromise matrix integrity. In some embodiments, an addition of an acid or peracid to a decellularization process can result in an increase in a flow rate and a decrease in an amount of time required for decellularization. In some embodiments, an addition of an acid or peracid to a decellularization process can result in an increase in a flow rate that can be maintained following the removal of the peracid from the solution or use of a non peracid solution. In some embodiments, a decellularization process can result in a purer decellularization (e.g., an increase in cell removal in less time). In some embodiments, a decellularization process can result in a purer decellularization (e.g., reduced residual DNA), as described herein. In some embodiments, an addition of a peracid to a solution or an increase in a flow rate during decellularization does not affect cellular adhesion properties. In some embodiments, an addition of a peracid (e.g., PAA) during decellularization does not affect cell adhesion during recellularization of a decellularized isolated organ, tissue, or portion thereof. In some embodiments, a decellularization using a peracid can result in complete removal of cells. In some embodiments, a decellularization using a peracid may not affect a stiffness or flexibility of an extracellular matrix. In some embodiments, a decellularization can be by perfusion. In some embodiments, a decellularization does not comprise agitation. In some embodiments, a decellularization using a peracid may not compromise integrity of a matrix (e.g., integrity of an ECM). In some embodiments, a decellularization using a peracid may not compromise flexibility, elasticity, or stiffness of a matrix (e.g., ECM). In some embodiments, a decellularization using a peracid may not result in a purer decellularization. In some embodiments, a decellularization using a peracid may not affect a surface property of an isolated organ, tissue, or portion thereof. In some embodiments, a decellularization using a peracid does not affect metabolic activity of an isolated organ, tissue, or portion thereof. In some embodiments, a decellularized isolated organ, tissue, or portion thereof can be introduced into a subject (e.g., a human). In some embodiments, a decellularized isolated organ, tissue, or portion thereof can be introduced into a subject after recellularization. In some embodiments, a decellularized isolated organ, tissue, or portion thereof can be introduced into a subject prior to recellularization. In some embodiments, human cells (e.g., stem cells, progenitor cells, regenerative cells, or isolated organ specific cells) can be introduced into an at least partially decellularized non-human mammal. Also disclosed herein in some embodiments, are methods or compositions (e.g., an at least partially decellularized or recellularized isolated organ, tissue, or portion thereof) which can provide for an improved rate of rejection as compared to another method of decellularization or a composition obtained from another method. In some embodiments, a rejection rate can be less than about or at most about 50%, 45%, 40%, 35%, 30%, 25%, 20%, 10%, 9%, 8%, 7%, 6%, 5%, 4%, 3%, 2%, 1%, or 0.5%. In some embodiments, a rejection rate can be decreased if an isolated organ, tissue, or portion thereof can be flushed or pretreated prior to decellularization. In some embodiments, a rejection rate can be decreased if an isolated organ, tissue, or portion thereof can be disinfected prior to decellularization. In some embodiments, a rejection rate can be decreased if an isolated organ, tissue, or portion thereof can be decellularized with a solution comprising a peracid, or an acid, or a peroxide.

Organ Recellularization

Also disclosed herein in some embodiments, are methods of at least partially recellularizing an at least partially decellularized isolated organs and portions thereof. In some embodiments, an isolated organ or tissue can be generated by contacting an at least partially decellularized isolated organ or tissue as described herein with a population of cells. In some embodiments, a population of cells can comprise a regenerative cell. In some embodiments, regenerative cells as used herein can be any cells used to recellularize an at least partially decellularized tissue, isolated organ or portion thereof. In some embodiments, regenerative cells can be totipotent cells, pluripotent cells, or multipotent cells, and can be uncommitted or committed. In some embodiments, regenerative cells also can be single-lineage cells. In some embodiments, regenerative cells can be undifferentiated cells, partially differentiated cells, or fully differentiated cells. In some embodiments, regenerative cells can comprise embryonic stem cells, progenitor cells, precursor cells, “adult”-derived stem cells including umbilical cord cells and fetal stem cells, or any combination thereof. In some embodiments, regenerative cells that can be used to recellularize an isolated organ or portion thereof disclosed herein can be, without limitation, embryonic stem cells, umbilical cord blood cells, tissue-derived stem or progenitor cells, bone marrow-derived stem or progenitor cells, blood-derived stem or progenitor cells, induced pluripotent stem cells (iPSCs), adipose tissue-derived stem or progenitor cells, mesenchymal stem cells (MSC), skeletal muscle-derived cells, or multipotent adult progenitor cells (MAPC). In some embodiments, additional regenerative cells that can be used include tissue-specific stem cells including cardiac stem cells (CSC), multipotent adult cardiac-derived stem cells, cardiac fibroblasts, cardiac microvasculature endothelial cells, or aortic endothelial cells. In some embodiments, bone marrow-derived stem cells such as bone marrow mononuclear cells (BM-MNC), endothelial or vascular stem or progenitor cells, and peripheral blood-derived stem cells such as endothelial progenitor cells (EPC) also can be used as regenerative cells. In some embodiments, a number of regenerative cells that can be introduced into an at least partially decellularized isolated organ or portion thereof in order to generate an isolated organ or tissue can be dependent on a size, weight, or type of an isolated tissue, organ or portion thereof, or a type or developmental stage of a regenerative cell that can be used. In some embodiments, different types of cells can have different tendencies as to a population density such cells will reach. In some embodiments, different isolated tissues, organs, or portions thereof, can be recellularized at different densities. In some embodiments, an at least partially decellularized isolated organ or tissue can be “seeded” with at least about 1,000 (e.g., at least 10,000, 100,000, 1,000,000, 10,000,000, or 100,000,000) regenerative cells; or can have from at or about 1,000 cells/mg tissue (wet weight, i.e., prior to decellularization) to at or about 10,000,000 cells/mg tissue (wet weight) attached thereto. In some embodiments, regenerative cells can be introduced (“seeded”) into an at least partially decellularized isolated organ or tissue by injection into one or more locations. In some embodiments, at least one type of cell (i.e., a cocktail of cells) can be introduced into an at least partially decellularized isolated organ or portion thereof. In some embodiments, a cocktail of cells or a population of cells can be injected at multiple positions in an at least partially decellularized isolated organ or tissue or different cell types can be injected into different portions of an at least partially decellularized isolated organ or portion thereof. In some embodiments, in addition to injection, regenerative cells, a population of cells, or a cocktail of cells can be introduced by perfusion into a cannulated decellularized isolated organ or portion thereof. In some embodiments, regenerative cells can be perfused into an at least partially decellularized isolated organ using a perfusion medium, which can then be changed to an expansion and/or differentiation medium to induce growth and/or differentiation of a regenerative cell. In some embodiments, during recellularization, an isolated organ or tissue can be maintained under conditions in which one or more regenerative cells can proliferate, multiply, differentiate, and any combination thereof, in an at least partially decellularized isolated organ or portion thereof. In some embodiments, those conditions can include, without limitation, an appropriate temperature, pressure, electrical activity, mechanical activity, force, an appropriate amounts of 02 and/or CO₂, an appropriate amount of humidity, sterile or near-sterile conditions, and any combination thereof. In some embodiments, during recellularization, an at least partially decellularized isolated organ or tissue and a regenerative cell attached thereto can be maintained in a suitable environment. In some embodiments, For example, a regenerative cell can require a nutritional supplement (e.g., nutrients and/or a carbon source such as glucose), exogenous hormones or growth factors, and/or a particular pH. In some embodiments, a growth factor can be at least one of: VEGF, DKK-1, FGF, BMP-1, BMP-4, SDF-1, IGF, HGF, an immune modulating agent such as a cytokine, a glucocorticoid, an IL2R antagonist, a leukotriene antagonist, or any combination thereof. In some embodiments, removing or reducing particulate in an isolated organ or portion thereof can improve recellularization. In some embodiments, engraftment, seeding, proliferation, survival, differentiation, and any combination thereof of cells introduced into an at least partially decellularized isolated organ or portion thereof can be improved by reducing a percent particulate of an isolated organ or portion thereof. In some embodiments, an amount or percent of engraftment, seeding, proliferation, survival, differentiation, and any combination thereof of cells introduced into an at least partially decellularized isolated organ or portion thereof can be improved by about 10%, 20%, 30%, 40%, 50%, 60%, 70%, 80%, 90%, 95%, or up to about 100%. In some embodiments, introducing a population of cells to an at least partially decellularized isolated organ or portion thereof disclosed herein can improve a percent viability of that population of cells as compared to a comparable population of cells that can be introduced to an otherwise comparable solid isolated organ or portion thereof that can be absent a reduced level of particulate. In some embodiments, particulate can increase a percent cytotoxicity thereby reducing a percent viability of cells. In some embodiments, viability can be from about 30%, 40%, 50%, 60%, 70%, 80%, 90%, 95%, or up to about 100% in a population of cells that has been introduced to an isolated organ or portion thereof that contains a reduced level of particulate. In some embodiments, regenerative cells as disclosed herein can be allogeneic to an at least partially decellularized isolated organ or portion thereof (e.g., a human decellularized isolated organ or tissue seeded with human regenerative cells), or regenerative cells can be xenogeneic to an at least partially decellularized isolated organ or portion thereof (e.g., a pig decellularized isolated organ or tissue seeded with human regenerative cells). “Allogeneic” as used herein refers to cells obtained from the same species as that from which an isolated organ or tissue originated (e.g., self (i.e., autologous) or related or unrelated individuals), while “xenogeneic” as used herein refers to cells obtained from a species different than that from which an isolated organ or tissue originated.

Uses of Isolated Organs and Portions Thereof

Also disclosed herein in some embodiments, are methods and uses of decellularized and recellularized isolated organs or portions thereof in a variety of applications. In some embodiments, isolated organs or portions thereof can be implanted into a subject. In some embodiments, a composition as described herein, such as an isolated organ or portion thereof, can be transplanted into a subject that has a disease. In some embodiments, relevant diseases that can require isolated organ transplantation include but are not limited to: isolated organ failure, cardiomyopathy, cirrhosis, chronic obstructive pulmonary disease, pulmonary edema, biliary atresia, emphysema and pulmonary hypertension, coronary heart disease, valvular heart disease, congenital heart disease, coronary artery disease, pancreatitis, cystic fibrosis, diabetes, hepatitis, hypertension, idiopathic pulmonary fibrosis, polycystic kidneys, short gut syndrome, injury, birth defects, genetic diseases, autoimmune disease, and any combination thereof. In some embodiments, implants can be used to replace or augment existing tissue, for example, to treat a subject with a kidney disorder by replacing a dysfunctional kidney of a subject with an exogenous or engineered kidney. In some embodiments, a subject can be monitored after implantation of an exogenous kidney, for amelioration of a kidney disorder. In some embodiments, a decellularized isolated organ or portion thereof disclosed herein can be utilized for implantation into a subject. In some embodiments, a composition disclosed herein, such as a solid isolated organ or portion thereof can have from about 1% to about 100% of its native function after decellularization. In some embodiments, a composition disclosed herein, such as a solid isolated organ or portion thereof can have from about 5%, 10%, 20%, 30%, 40%, 50%, 60%, 70%, 80%, 90%, 95%, or up to about 100% of its native function after decellularization. In some embodiments, particular isolated organs or portions thereof can be suitable for transplantation when they function below that of their native counterpart. In some embodiments, for example, a liver and a kidney can need approximately from about 20% of a total organ function to save a person from liver failure or remove them from dialysis. In some embodiments, a liver and kidney can need approximately from about 20-30%, 30-40%, 20-50%, 20-60%, 40-60% of a total organ function to be suitable for transplantation. In some embodiments, an isolated organ can function equal to a native counterpart. In some embodiments, for example, a heart can be more complicated, in that, it can need from about 80%, 85%, 90%, 95%, 96%, 97%, 98%, 99%, or up to about 100% function at a time of transplantation. In some embodiments, an isolated organ such as a pancreas or lung can function and be transplantable from 15%, 20%, 30%, 40%, or 50% of native function. In some embodiments, a pancreas can function and be transplantable from 10% or more. In some embodiments, an isolated organ disclosed herein can be used as an accessory organ to help with native function. In some embodiments, a lifespan of a subject can be extended after transplantation of a composition, such as an isolated organ or portion thereof disclosed herein. In some embodiments, for example, a lifespan of a subject can be extended from about 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, 15 years, 20 years, 30 years, 40 years, 50 years, 60 years, 70 years, 80 years, 90 years, or up to about 100 years after transplantation. In some embodiments, transplantation of a composition, such as an isolated organ or portion thereof disclosed herein, can reduce a need for a secondary treatment in a subject. In some embodiments, secondary treatments can refer to dialysis, pacemakers, respirators, and combinations thereof. In some embodiments, at least partially decellularized isolated organs or portions thereof or at least partially recellularized isolated organs or portions thereof can be used in vitro to screen a wide variety of compounds, for effectiveness and cytotoxicity of pharmaceutical agents, chemical agents, growth factors, or regulatory factors. In some embodiments, a culture can be maintained in vitro and exposed to a compound to be tested. In some embodiments, an activity of a cytotoxic compound can be measured by its ability to damage or kill cells in culture. In some embodiments, this can readily be assessed by vital staining techniques. In some embodiments, an effect of growth or regulatory factors can be assessed by analyzing a cellular content of a matrix, e.g., by total cell counts, and differential cell counts. In some embodiments, this can be accomplished using standard cytological and/or histological techniques including a use of immunocytochemical techniques employing antibodies that define type-specific cellular antigens. In some embodiments, an effect of various drugs on normal cells cultured in a reconstructed artificial isolated organ can be assessed. In some embodiments, decellularized and recellularized isolated organs or portions thereof which can be used in vitro to filter aqueous solutions, for example, a reconstructed artificial kidney can be used to filter blood. In some embodiments, using a reconstructed kidney provides a system with morphological features that resemble an in vivo kidney product, which in some embodiments, can be suitable for hemodialysis, or for hemofiltration to remove water and low molecular weight solutes from blood. In some embodiments, an artificial kidney can be maintained in vitro and exposed to blood which can be infused into a luminal side of an artificial kidney. In some embodiments, a processed aqueous solution can be collected from an abluminal side of an engineered kidney. In some embodiments, an efficiency of filtration can be assessed by measuring an ion, or metabolic waste content of a filtered and unfiltered blood. In some embodiments, decellularized and recellularized isolated organs or portions thereof can be used as a vehicle for introducing genes or gene products in vivo to assist or improve a result of a transplantation, or for use in gene therapies. In some embodiments, cultured cells, such as endothelial cells, can be engineered to express gene products. In some embodiments, cells can be engineered to express gene products transiently or under inducible control, or as a chimeric fusion protein anchored to an endothelial cell, for example, a chimeric molecule composed of an intracellular and/or transmembrane domain of a receptor or receptor-like molecule, fused to a gene product as an extracellular domain. In some embodiments, one or more endothelial cells can be genetically engineered to express a gene for which a patient can be deficient, or which would exert a therapeutic effect. In some embodiments, a gene of interest engineered into an endothelial cell or parenchyma cell can be related to a disease being treated. In some embodiments, for example for a kidney disorder, an endothelial or cultured kidney cell can be engineered to express gene products that can ameliorate a kidney disorder. In some embodiments, at least two populations of cells can be introduced into an at least partially decellularized isolated organ or portion thereof. In some embodiments, isolated organs that can be engineered include, but are not limited to, heart, kidney, liver, pancreas, spleen, bladder, uterus, ureter, urethra, skeletal muscle, small and large bowel, esophagus, stomach, brain, spinal cord and bone. Other embodiments and uses will be apparent to those skilled in the art from consideration of the specification and practice of the disclosed herein.

In some embodiments, decellularized isolated organs or portions thereof can be utilized as acellular compositions. Disclosed herein can be an acellular or substantially acellular isolated organ or portion thereof.

In some embodiments, a method of an isolated organ transplantation can comprise compressing an isolated organ or portion thereof. The ability to compress an isolated organ or portion thereof can be influenced by the particulate percentage. For an isolated organ or portion thereof containing over 35% particulate can be more difficult to compress as compared to an isolated organ or portion thereof containing 35% particulate or less than 35% particulate. For an isolated organ or portion thereof containing over 50% particulate can be more difficult to compress as compared to an isolated organ or portion thereof containing 50% particulate or less than 50% particulate.

### Kits

Disclosed herein can be kits comprising at least partially decellularized mammalian isolated organs or portions thereof. In some embodiments, a kit can include an at least partially decellularized isolated organ or portion thereof and instructions for use thereof. In some embodiments, a kit can comprise a sterile container which can contain the at least partially decellularized isolated organ or portion thereof; such containers can be boxes, ampules, bottles, vials, tubes, bags, pouches, blister-packs, or other suitable container forms known in the art. Such containers can be made of plastic, glass, laminated paper, metal foil, or other materials suitable for holding isolated organs or portions thereof. In some embodiments, a delivery vehicle composition can be dehydrated, stored and then reconstituted such that a substantial portion of an internal content can be retained. In some embodiments, the kit can comprise a compressed packaging containing an at least partially decellularized isolated organ or portion thereof. For example, a sterile pouch containing an isolated organ or portion thereof can be degassed or otherwise compressed into a sheet.

In some embodiments, disclosed herein can be a system comprising any of the compositions disclosed herein. In some embodiments, a system can comprise at least one of a pump, housing, tubing, incubator, motor, computer, storage medium, or any combination thereof.

### SPECIFIC EMBODIMENTS

A number of methods and systems are disclosed herein. Specific exemplary embodiments of these methods and systems are disclosed below.

### Embodiment 1

A method comprising contacting an isolated organ or portion thereof with a liquid, wherein: (a) the liquid can be at least partially degassed; (b) the liquid can comprise a peracid;
(c) the isolated organ or portion thereof can be from a mammal that was fed within about ten hours prior to removal of the isolated organ or portion thereof; or (d) any combination of (a)-(c).

### Embodiment 2

The method of embodiment 1, wherein the method can comprise at least two of: (a), (b) or (c).

### Embodiment 3

The method of embodiment 1 or embodiment 2, wherein the method can comprise (a), (b), and (c).

### Embodiment 4

The method of any one of embodiments 1-3, wherein the isolated organ or portion thereof can be from a mammal.

### Embodiment 5

The method of embodiment 4, wherein the mammal can be a non-human mammal.

### Embodiment 6

The method of embodiment 5, wherein the non-human mammal can be a pig, a sheep, a goat, a cow, a dog, a cat, or a monkey.

### Embodiment 7

The method of embodiment 4, wherein the mammal can be a human mammal.

### Embodiment 8

The method of any one of embodiments 1-7, wherein the isolated organ or portion thereof can be at least part of a liver, a lung, a heart, a kidney a bladder, a pancreas, a spleen, a uterus, a portion of any of these, or any combination thereof.

### Embodiment 9

The method of any one of embodiments 1-8, wherein the contacting can comprise perfusing the liquid in at least a portion of the isolated organ or portion thereof, injecting the liquid in at least a portion of the isolated organ or portion thereof, spraying the liquid on at least a portion of the isolated organ or portion thereof, submerging the isolated organ or portion thereof in the liquid, or any combination thereof.

### Embodiment 10

The method of embodiment 9, wherein the contacting can comprise the perfusing the liquid in at least the portion of the isolated organ or portion thereof.

### Embodiment 11

The method of embodiment 10, wherein the isolated organ or portion thereof can be perfused with at least about 10 liters of the liquid.

### Embodiment 12

The method of embodiment 10, wherein the liquid can be continuously perfused through the isolated organ or portion thereof.

### Embodiment 13

The method of embodiment 10, wherein perfusion can be retrograde.

### Embodiment 14

The method of embodiment 10, wherein perfusion anterograde.

### Embodiment 15

The method of embodiment 10, wherein the liquid can be recirculated during perfusion.

### Embodiment 16

The method of any one of embodiments 1-15, wherein prior to, during, or after the contacting, the isolated organ or portion thereof can be cannulated.

### Embodiment 17

The method of any one of embodiments 1-16, wherein the liquid can comprise an at least partially degassed decellularization media, thereby forming an at least partially decellularized isolated organ or portion thereof.

### Embodiment 18

The method of embodiment 17, wherein the at least partially decellularized isolated organ or portion thereof can comprise an extracellular matrix.

### Embodiment 19

The method of embodiment 18, wherein the extracellular matrix can comprise: fibronectin, fibrillin, laminin, elastin, a collagen family protein, a glycosaminoglycan, a ground substance, a reticular fiber, thrombospondin, or any combination thereof.

### Embodiment 20

The method of embodiment 18, wherein the extracellular matrix can comprise a collagen family protein, wherein the collagen family protein can be collagen I, collagen II, collagen III, or collagen IV.

### Embodiment 21

The method of any one of embodiments 18-20, wherein the extracellular matrix can comprise a vasculature bed.

### Embodiment 22

The method of embodiment 21, wherein the vasculature bed can be intact.

### Embodiment 23

The method of any one of embodiments 18-22, wherein the liquid can be at least partially degassed and wherein the extracellular matrix has an increased compressive modulus relative to an otherwise comparable extracellular matrix produced using a non-degassed liquid.

### Embodiment 24

The method of any one of embodiments 1-23, wherein the method can comprise at least (a).

### Embodiment 25

The method of embodiment 24, wherein the isolated organ or portion thereof can be contacted by the at least partially degassed liquid for at least about two hours.

### Embodiment 26

The method of embodiment 24, wherein a dissolved gas in the at least partially degassed liquid has a concentration of less than about 1 milligram (mg) per liter (L).

### Embodiment 27

The method of embodiment 26, wherein the concentration of the dissolved gas can be measured using a static headspace gas chromatography or a dynamic luminescence quenching.

### Embodiment 28

The method of embodiment 26, wherein the dissolved gas can be selected from the group consisting of: oxygen, nitrogen, carbon monoxide, carbon dioxide, a noble gas, and any combination thereof.

### Embodiment 29

The method of embodiment 24, wherein prior to the contacting, a liquid can be exposed to an air pressure of less than about 700 torr, thereby forming the at least partially degassed liquid.

### Embodiment 30

The method of embodiment 24, wherein prior to the contacting, a liquid can be exposed to an air pressure of less than about 1 torr, thereby forming the at least partially degassed liquid.

### Embodiment 31

The method of embodiment 29, wherein the liquid can be exposed to the air pressure of less than about 700 torr for at least about ten minutes prior to contacting, thereby forming the at least partially degassed liquid.

### Embodiment 32

The method of embodiment 24, wherein prior to the contacting, a liquid can be exposed to a temperature of from about 10° C. to about 100° C. for at least two hours prior to contacting, thereby forming the at least partially degassed liquid.

### Embodiment 33

The method of embodiment 24, wherein prior to the contacting, a liquid can be exposed to sonication for at least about one second, thereby forming the at least partially degassed liquid.

### Embodiment 34

The method of embodiment 24, wherein prior to the contacting, a liquid can be sparged for at least about one second to form the at least partially degassed liquid.

### Embodiment 35

The method of embodiment 34, wherein the liquid can be sparged with a chemically inert gas.

### Embodiment 36

The method of embodiment 35, wherein the chemically inert gas can comprise: nitrogen, argon, helium, or any combination thereof.

### Embodiment 37

The method of embodiment 24, wherein prior to contacting, a liquid can be contacted with a gas-liquid separation membrane, thereby forming the at least partially degassed liquid.

### Embodiment 38

The method of embodiment 24, wherein prior to the contacting, a liquid can be contacted with a reductant, thereby forming the at least partially degassed liquid.

### Embodiment 39

The method of embodiment 38, wherein the reductant can be ammonium sulfite.

### Embodiment 40

The method of embodiment 24, wherein prior to the contacting, a liquid can be degassed through freeze-pump-thawing, thereby forming the at least partially degassed liquid.

### Embodiment 41

The method of embodiment 24, wherein the isolated organ or portion thereof after the contacting can comprise (a) fewer air emboli, (b) fewer microbubbles, (c) less pigmentation, or (d) any combination thereof relative to an otherwise comparable isolated organ or portion thereof produced by contacting for a comparable amount of time with an otherwise comparable liquid that has not been at least partially degassed.

### Embodiment 42

The method of embodiment 24, wherein the at least partially degassed liquid can comprise an at least partially degassed decellularization media, thereby forming an at least partially decellularized isolated organ or portion thereof.

### Embodiment 43

The method of embodiment 42, wherein the at least partially decellularized isolated organ or portion thereof after the contacting with the at least partially degassed decellularization media contains fewer cells than an otherwise comparable isolated organ or portion thereof decellularized with an otherwise comparable non-degassed decellularization media for a comparable period of time.

### Embodiment 44

The method of embodiment 42, wherein an amount of time sufficient to produce the at least partially decellularized isolated organ or portion thereof with the at least partially degassed decellularization media can be less than an amount of time to produce a substantially decellularized isolated organ or portion thereof with a non-degassed decellularization media.

### Embodiment 45

The method of embodiment 42, wherein the at least partially degassed decellularization media can comprise an acid, a base, a hypertonic solution, a hypotonic solution, a detergent, a surfactant, an alcohol, a biological reagent, a salt, or any combination thereof.

### Embodiment 46

The method of embodiment 45, wherein the at least partially degassed decellularization media can comprise the surfactant, and wherein the surfactant can comprise an ionic surfactant.

### Embodiment 47

The method of embodiment 46, wherein the ionic surfactant can comprise an alkyl sulfate.

### Embodiment 48

The method of embodiment 47, wherein the alkyl sulfate can comprise sodium dodecyl sulfate.

### Embodiment 49

The method of embodiment 47, wherein the alkyl sulfate can comprise: sodium dodecyl sulfate, ammonium dodecyl sulfate, sodium lauryl sulfate, sodium octyl sulfate, sodium 2-ethylhexyl sulfate, lithium dodecyl sulfate, potassium lauryl sulfate, or any combination thereof.

### Embodiment 50

The method of any one of embodiments 1-49, further comprising cannulating the isolated organ or portion thereof, thereby generating a cannulated isolated organ or portion thereof.

### Embodiment 51

The method of embodiment 50, wherein the liquid can be perfused into the cannulated isolated organ or portion thereof.

### Embodiment 52

The method of embodiment 51, wherein liquid perfusing through the isolated organ or portion thereof can be at a pressure from about 10 MPa to about 1000 MPa.

### Embodiment 53

The method of embodiment 51 or 52, wherein liquid perfusing through the isolated organ or portion thereof can be at a flow rate from about 100 milliliter (mL) per minute to about 100 liters (L) per minute.

### Embodiment 54

The method of any one of embodiments 1-23, wherein the method can comprise at least (b).

### Embodiment 55

The method of embodiment 54, wherein the peracid can comprise: peroxyacetic acid, peroxyoctanoic acid, a sulfoperoxycarboxylic acid, peroxysulfonated oleic acid, peroxyformic acid, peroxyoxalic acid, peroxypropanoic acid, peroxybutanoic acid, peroxypentanoic acid, peroxyhexanoic acid, peroxyadipic acid, peracetic acid, perlactic acid, peroxycitric, peroxybenzoic acid, or any combination thereof.

### Embodiment 56

The method of embodiment 55, wherein the peracid can comprise the peracetic acid.

### Embodiment 57

The method of embodiment 54, wherein the peracid can comprise a peroxy acid.

### Embodiment 58

The method of embodiment 57, wherein peroxy acid can be present in the liquid at a concentration of from about 0.1% to about 1.0% by weight.

### Embodiment 59

The method of embodiment 57, wherein peroxy acid can be present in the liquid at a concentration of from about 0.1% to about 1.0% by volume.

### Embodiment 60

The method of embodiment 57, wherein the peroxy acid can be present in the liquid at a concentration of from about 40 parts per million (ppm) to about 700 ppm.

### Embodiment 61

The method of any one of embodiments 54-50, wherein the contacting produces an at least partially decellularized isolated organ or portion thereof.

### Embodiment 62

The method of embodiment 61, wherein the at least partially decellularized isolated organ or portion thereof can comprise an extracellular matrix comprising an exterior surface and a vascular tree.

### Embodiment 63

The method of any one of embodiments 54-62, further comprising cannulating the isolated organ or portion thereof, thereby generating a cannulated isolated organ or portion thereof.

### Embodiment 64

The method of embodiment 63, wherein the liquid can be perfused into the cannulated isolated organ or portion thereof.

### Embodiment 65

The method of any one of embodiments 61-63, wherein the at least partially decellularized isolated organ or portion thereof can comprise a reduced amount of cells as compared to an otherwise comparable decellularized isolated organ or portion thereof contacted with an otherwise comparable liquid that does not comprise a peracid for a comparable period of time.

### Embodiment 66

The method of embodiment 65, wherein the reduced amount of cells can comprise a reduction of at least about 85%, at least about 90%, at least about 92%, at least about 95%, at least about 97%, at least about 98%, at least about 99%, or at least about 99.5% of cells from the isolated organ or portion thereof.

### Embodiment 67

The method of embodiment 64, wherein a flow rate of the perfusion can be increased compared to a flow rate of a perfusion of an otherwise comparable liquid that does not comprise peracid.

### Embodiment 68

The method of embodiment 67, wherein the flow rate can be increased without substantially compromising an integrity of extracellular matrix of the isolated organ or portion thereof.

### Embodiment 69

The method of embodiment 67, wherein the flow rate can be increased above a physiological rate.

### Embodiment 70

The method of embodiment 69, wherein the flow rate can be increased above the physiological rate by about 5%, 10%, 15%, 20%, 25%, 30%, 35%, 40%, 45%, 50%, 55%, 60%, 70%, 80%, 90%, 95% or 99%.

### Embodiment 71

The method of any one of embodiments 1-23, wherein the method can comprise at least (c).

### Embodiment 72

The method of embodiment 71, wherein the isolated organ or portion thereof can comprise an extracellular matrix comprising an exterior surface and a vascular tree.

### Embodiment 73

The method of embodiment 72, further comprising cannulating the isolated organ or portion thereof, thereby generating a cannulated isolated organ or portion thereof.

### Embodiment 74

The method of embodiment 73, wherein the liquid can be perfused into the cannulated isolated organ or portion thereof.

### Embodiment 75

The method of any of embodiments 71-74, wherein the isolated organ or portion thereof can be subsequently contacted with a wash media.

### Embodiment 76

The method of embodiment 75, wherein contacting with the wash media can be by perfusion, injection, submersion, or any combination thereof.

### Embodiment 77

The method of embodiment 76, wherein the contacting with the wash media can be by perfusion.

### Embodiment 78

The method of embodiment 76, wherein the contacting with the wash media can be by submersion.

### Embodiment 79

The method of any one of embodiments 75-78, wherein the wash media can comprise a pH buffer.

### Embodiment 80

The method of embodiment 79, wherein the pH buffer can comprise a phosphate.

### Embodiment 81

The method of any one of embodiments 75-80, wherein the wash media can comprise saline.

### Embodiment 82

The method of embodiment 81, wherein the saline can be at a concentration of from about 0.1% to about 10%.

### Embodiment 83

The method of any one of embodiments 71-82, wherein the isolated organ or portion thereof can comprise a reduced particulate level as compared to an otherwise comparable isolated organ or portion thereof produced from an animal which had not been fed within about 10 hours prior to removal of the isolated organ or portion thereof from the animal.

### Embodiment 84

The method of embodiment 83, wherein the reduced particulate level can be reduced by at least about: 90%, 85%, 80%, 75%, 70%, 65%, 60%, 55%, 50%, 45%, 40%, 35%, 30%, 25%, 20%, 15%, 10%, 5%, 1% as compared to an otherwise comparable isolated organ or portion thereof produced from an animal which had not been fed within about 10 hours prior to removal of the isolated organ or portion thereof from the animal.

### Embodiment 85

The method of embodiment 83 or 84, wherein the particulate level can be determined by visual examination, determining an outflow, determining turbidity, microscopy analysis, or any combination thereof.

### Embodiment 86

The method of any one of embodiments 1-85, further comprising washing the isolated organ or portion thereof with a wash media prior to the contacting.

### Embodiment 87

The method of embodiment 86, wherein the wash media can be a hypertonic solution.

### Embodiment 88

The method of embodiment 87, wherein the hypertonic solution can comprise saline.

### Embodiment 89

The method of embodiment 86, wherein the wash media can comprise a disinfecting solution.

### Embodiment 90

The method of embodiment 8940, wherein washing with the disinfection solution can comprise placing the isolated organ or portion thereof in a disinfection bath comprising the disinfecting solution.

### Embodiment 91

The method of embodiment 89 or 90, wherein the disinfecting solution can comprise: a peracid, hydrogen peroxide, acetic acid, peracetic acid (PAA), saline, sodium dodecyl sulfate, sodium hydroxide (NaOH), or any combination thereof.

### Embodiment 92

The method of embodiment 89 or 90, wherein the disinfecting solution can comprise a peroxy acid.

### Embodiment 93

The method of embodiment 92, wherein the peroxy acid can comprise: peroxyacetic acid, peroxyoctanoic acid, a sulfoperoxycarboxylic acid, peroxysulfonated oleic acid, peroxyformic acid, peroxyoxalic acid, peroxypropanoic acid, peroxybutanoic acid, peroxypentanoic acid, peroxyhexanoic acid, peroxyadipic acid, peracetic acid, perlactic acid, peroxycitric, peroxybenzoic acid, or any combination thereof.

### Embodiment 94

The method of embodiment 92, wherein the peroxy acid can be peracetic acid.

### Embodiment 95

The method of any one of embodiments 92-94, wherein peroxy acid can be present at a concentration of from about 0.1% to about 1.0% by weight.

### Embodiment 96

The method of any one of embodiments 92-94, wherein peroxy acid can be present at a concentration of from about 0.1% to about 1.0% by volume.

### Embodiment 97

The method of any one of embodiments 92-94, wherein the peroxy acid can be present at a concentration of from about 40 ppm to about 700 ppm.

### Embodiment 98

The method of any one of embodiments 1-97, after the contacting with the liquid, further contacting the isolated organ or portion thereof with a second liquid.

### Embodiment 99

The method of embodiment 98, wherein the second liquid can be an at least partially degassed liquid.

### Embodiment 100

The method of embodiment 99, wherein the second liquid can comprise a wash media.

### Embodiment 101

The method of embodiment 100, wherein the wash media can comprise distilled water.

### Embodiment 102

The method of embodiment 100, wherein the wash media can comprise: distilled water, reverse osmosis water, filtered water, HEPC-treated water, purified water, deionized water, saline, or any combination thereof.

### Embodiment 103

The method of embodiment 99, wherein the at least partially decellularized isolated organ or portion thereof contains less than about 1% weight per weight of detergent after contacting of the at least partially decellularized isolated organ or portion thereof with the second liquid.

### Embodiment 104

The method of embodiment 99, wherein the at least partially decellularized isolated organ or portion thereof contains less than about 0.10% weight per weight of detergent after contacting of the at least partially decellularized isolated organ or portion thereof with the second liquid.

### Embodiment 105

The method of embodiment 99, wherein after the contacting with the second liquid, blood flows through a greater volume of vasculature of the at least partially decellularized isolated organ or portion thereof as compared to an otherwise comparable at least partially decellularized isolated organ or portion thereof produced using a non-degassed second liquid.

### Embodiment 106

The method of any one of embodiments 1-106, further comprising contacting the at least partially decellularized isolated organ or portion thereof with a regeneration media.

### Embodiment 107

The method of embodiment 106, wherein the regeneration media can be an at least partially degassed cellular regeneration media.

### Embodiment 108

The method of embodiment 107, wherein the contacting with the at least partially degassed cellular regeneration media forms an at least partially recellularized organ or portion thereof.

### Embodiment 109

The method of embodiment 107, wherein the at least partially degassed cellular regeneration media can comprise a population of regenerative cells.

### Embodiment 110

The method of embodiment 109, wherein the population of regenerative cells can comprise totipotent cells, pluripotent cells, multipotent cells, or any combination thereof.

### Embodiment 111

The method of embodiment 109, wherein the population of regenerative cells can comprise undifferentiated cells, partially differentiated cells, fully differentiated cells, or any combination thereof.

### Embodiment 112

The method of embodiment 109, wherein the population of regenerative cells comprise embryonic stem cells, umbilical cord blood cells, induced pluripotent stem cells (iPSCs), tissue-derived stem or progenitor cells, bone marrow-derived stem or progenitor cells, blood-derived stem or progenitor cells, mesenchymal stem cells (MSC), skeletal muscle-derived cells, multipotent adult progenitor cells (MAPC), cardiac stem cells (CSC), multipotent adult cardiac-derived stem cells, cardiac fibroblasts, cardiac microvasculature endothelial cells, aortic endothelial cells, bone marrow mononuclear cells (BM-MNC), endothelial progenitor cells (EPC), or any combination thereof.

### Embodiment 113

The method of any one of embodiments 109-112, wherein the population of regenerative cells can be independently from a mammal.

### Embodiment 114

The method of embodiment 113, wherein the mammal can be a rodent, a pig, a rabbit, cattle, a sheep, a dog, or a human.

### Embodiment 115

The method of any one of embodiments 109-114, wherein the population of regenerative cells can comprise at least about 1,000 cells.

### Embodiment 116

The method of any one of embodiment 109-114, wherein an amount of the population of regenerative cells can be at least about 1,000 regenerative cells per milligram (mg) of the at least partially decellularized isolated organ or portion thereof.

### Embodiment 117

The method of embodiment 107, wherein the at least partially degassed cellular regeneration media can comprise a growth factor.

### Embodiment 118

The method of embodiment 117, wherein the growth factor can comprise: VEGF, DKK-1, FGF, BMP-1, BMP-4, SDF-1, EGF, IGF, HGF, or any combination thereof.

### Embodiment 119

The method of embodiment 107, wherein the at least partially degassed cellular regeneration media can comprise an immune modulating agent.

### Embodiment 120

The method of embodiment 119, wherein the immune modulating agent can comprise: a cytokine, a glucocorticoid, an IL-2R antagonist, a leukotriene antagonist, or any combination thereof.

### Embodiment 121

The method of embodiment 107, wherein the contacting with the at least partially degassed cellular regeneration media results in a higher degree of recellularization relative to an otherwise comparable isolated organ or portion thereof produced using a non-degassed cellular regeneration media.

### Embodiment 122

The method of embodiment 108, wherein the at least partially recellularized organ or portion thereof can comprise at least one human cell.

### Embodiment 123

An at least partially decellularized isolated organ or portion thereof generated by the method of any one of embodiments 1-105.

### Embodiment 124

The at least partially decellularized isolated organ or portion thereof of embodiment 123, wherein the isolated organ or portion thereof contains an at least partially degassed solution.

### Embodiment 125

A method of reducing cytotoxicity of a cell during recellularization of an at least partially decellularized isolated organ or portion thereof comprising introducing a population of cells to the at least partially decellularized isolated organ or portion thereof of embodiment 123.

### Embodiment 126

An at least partially recellularized mammalian isolated organ or portion thereof generated by the method of any one of embodiments 106-122.

### Embodiment 127

The at least partially recellularized mammalian isolated organ or portion thereof of embodiment 126 comprising a cellular regeneration media with less than about 8 parts per million (ppm) of oxygen when stored at a temperature of about 25° C.

### Embodiment 128

A system comprising (a) the at least partially decellularized isolated organ or portion generated by the method of any one of embodiments 1-122, (b) a device for moving fluid, (c) a cannula, and (d) a port.

### Embodiment 129

The system of embodiment 128, wherein the system further can comprise degassed decellularization media.

### Embodiment 130

The system of embodiment 128, wherein the system further can comprise degassed wash media.

### Embodiment 131

The system of embodiment 128, wherein the system further can comprise an at least partially enclosed container.

### Embodiment 132

The system of embodiment 128, wherein a flow of at least partially degassed solution at a flow rate at or about 100 ml per minute to at or about 100 liters per minute can be passing through the isolated organ or portion thereof.

### Embodiment 133

The system of embodiment 128, wherein the at least partially degassed solution can be passing through the isolated organ or portion thereof at a flow rate at or about 100 ml per minute to at or about 100 liters per minute.

### Embodiment 134

The system of embodiment 128, further comprising a peristaltic pump.

### Embodiment 135

A kit comprising: (a) the at least partially decellularized isolated organ or portion thereof generated by the method of any one of embodiments 1-122, (b) at least one cannula, (c) reagents, (d) instructions for recellularizing the at least partially decellularized isolated organ or portion thereof, (e) instructions for introducing the at least partially recellularized isolated organ or portion thereof in a subject, or (f) any combination thereof.

### Embodiment 133

The kit of embodiment 135, wherein the at least partially decellularized isolated organ or portion thereof can be in a packaging.

### Embodiment 134

The kit of embodiment 136, wherein the packaging can be sterile.

### Embodiment 135

A kit comprising the at least partially decellularized isolated organ or portion thereof of embodiment 135.

### EXAMPLES

### Example 1: Perfusion Decellularization of Isolated Solid Organs Using a Degassed Media

### Part 1: Cannulation

Hearts are systemically heparinized with 400 U of heparin/kg of donor for example pig, canine, human, primate, and sheep. Following heparinization, the heart and the adjacent large vessels are carefully removed. The heart can be placed in a physiologic saline solution (0.9%) that can contain heparin (2000 U/ml) and held at 5° C. until further processing. Under sterile conditions, the connective tissue can be removed from the heart and the large vessels. The inferior venae cava and the left and right pulmonary veins can be ligated distal from the right and left atrium using monofil, non-resorbable ligatures.

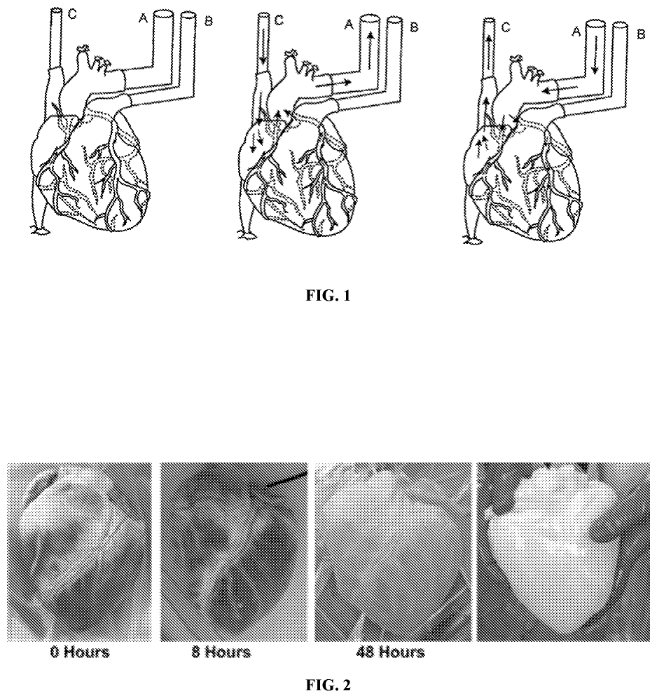

The heart can be mounted on a decellularization apparatus for perfusion FIG. 1. The descending thoracic artery can be cannulated to allow retrograde coronary perfusion (FIG. 1, Cannula A). The branches of the thoracic artery (e.g., brachiocephalic trunc, left common carotid artery, left subclavian artery) are ligated. The pulmonary artery can be cannulated before its division into the left and right pulmonary artery (FIG. 1, Cannula B). The superior vena cava can be cannulated (FIG. 1, Cannula C). This configuration allows for both retrograde and antegrade coronary perfusion. When positive pressure can be applied to the aortic cannula (A), a perfusion occurs from the coronary arteries through the capillary bed to the coronary venous system to the right atrium and the superior caval vein (C). Perfusion can occur from the right atrium, the coronary sinus, and the coronary veins through the capillary bed to the coronary arteries and the aortic cannula (A) during application of positive pressure to the superior vena cava cannula (C).

### Part II: Decellularization

### Basic Protocol

After mounting of a heart on a decellularization apparatus, antegrade perfusion can be started with cold, heparinized, calcium-free phosphate buffered solution containing 1-5 mmol adenosine per L perfusate to reestablish constant coronary flow. Prior to perfusion, a buffered solution can be placed under high vacuum for 30 minutes with vigorous stirring using a stir bar for 30 minutes at 25° C.

Coronary flow can be assessed by measuring the coronary perfusion pressure and the flow, and calculating coronary resistance. After 15 minutes of stable coronary flow, the detergent-based decellularization process can be initiated utilizing degassed decellularization solution. The heart can be perfused antegrade with a degassed detergent solution. After perfusion, a heart can be flushed with a degassed buffer (e.g., PBS) retrograde. The heart can then be perfused with a degassed PBS buffer containing antibiotics and then a degassed PBS buffer containing DNase I. The heart can then be perfused with 1% benzalkonium chloride to reduce microbial contamination and to prevent future microbial contamination, and then perfused with PBS to wash the isolated organ of any residual cellular components, enzymes, or detergent.

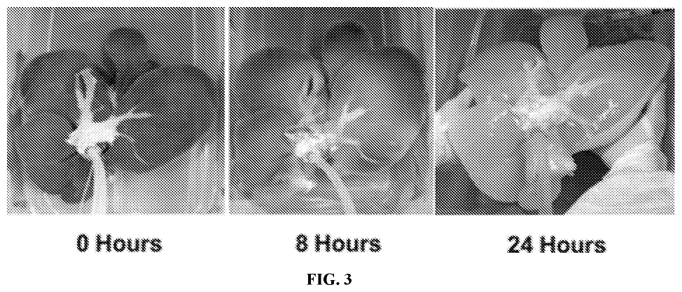

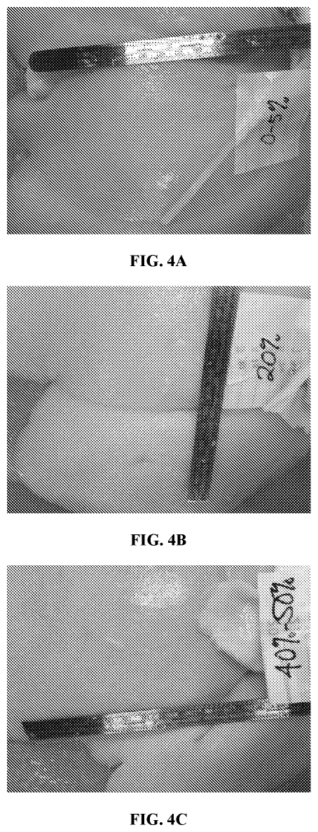

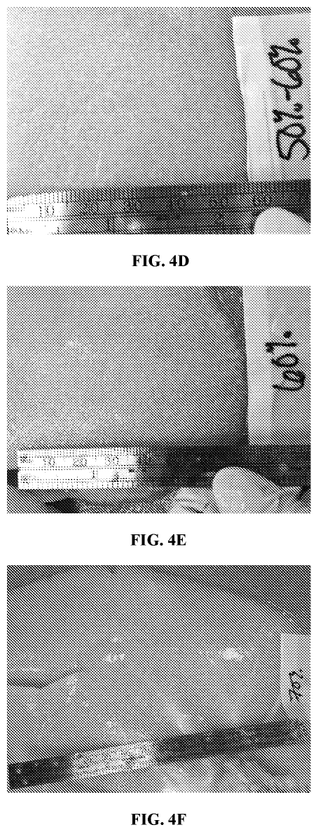

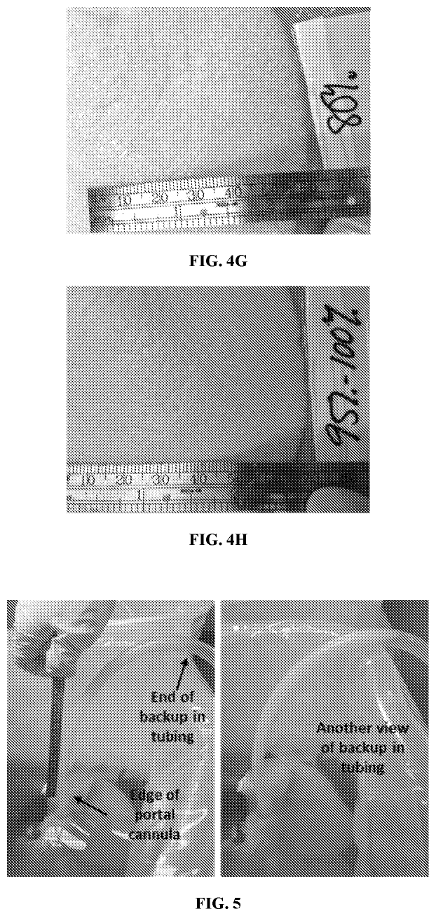

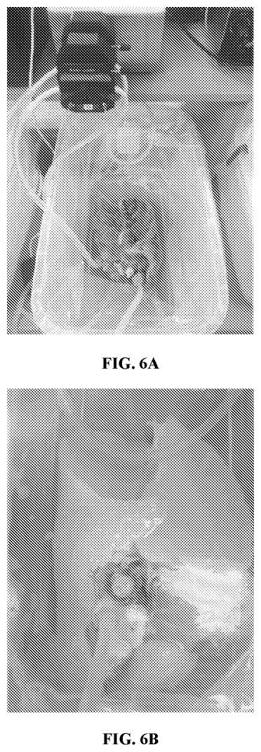

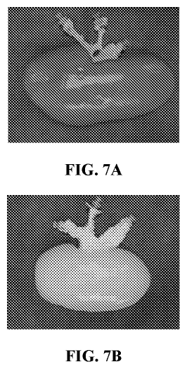

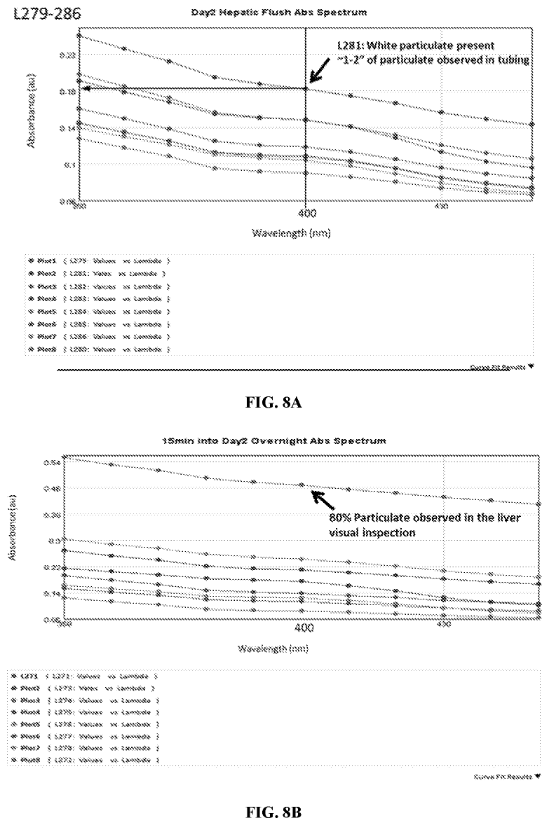

Exemplary solid organs that were decellularized are shown in FIG. 2 (porcine heart) and FIG. 3 (porcine liver).

Decellularization with the degassed buffers produces fewer microbubbles than decellularization with non-degassed buffers.

### PEG Decellularization

Hearts are washed in 200 ml PBS containing 100 U/ml penicillin, 0.1 mg/ml Streptomycin, and 0.25 μg/ml Amphotericin B with no recirculation. Hearts are then decellularized with 35 ml of degassed polyethyleneglycol (PEG; 1 g/ml) for up to 30 minutes with manual recirculation. The degassed polyethyleneglycol was prepared by placing the polyethyleneglycol solution under a vacuum pump as described above. The isolated organ can then be washed with 500 ml degassed PBS for up to 24 hours using a pump for recirculation. The washing step can be repeated at least twice for at least 24 hours each time. Hearts are exposed to 35 ml DNase I (70 U/ml) for at least 1 hour with manual recirculation. The isolated organs are washed again with 500 ml degassed PBS for at least 24 hours.

Decellularization with the degassed buffers produces more efficient decellularization of the isolated organ when pumped for 30 minutes relative to using non-degassed buffers.

### Triton X and Trypsin Decellularization

Hearts are washed in 200 ml PBS containing 100 U/ml Penicillin, 0.1 mg/ml Streptomycin, and 0.25 μg/ml Amphotericin B for at least about 20 minutes with no recirculation. Hearts are then decellularized with a degassed aqueous solution of 0.05% Trypsin for 30 min followed by perfusion with 500 ml degassed PBS containing 5% Triton-X and 0.1% ammonium-hydroxide for about 6 hours. Both solutions are prepared by dissolving a detergent with water previously degassed. Hearts are perfused with degassed deionized water for about 1 hour, and then perfused with degassed PBS for 12 h. Hearts are then washed 3 times for 24 hours each time in 500 ml degassed PBS using a pump for recirculation. The hearts are perfused with 35 ml DNase I (70 U/ml) for 1 hour with manual recirculation and washed twice in 500 ml PBS for at least about 24 hours each time using a pump for recirculation.

Decellularization with the degassed buffers produces more efficient decellularization of the isolated organ when pumped for 30 minutes relative to using non-degassed buffers.

### SDS Decellularization

Hearts are washed in 200 ml PBS containing 100 U/ml Penicillin, 0.1 mg/ml Streptomycin, and 0.25 μg/ml Amphotericin B for at least about 20 mins with no recirculation. The hearts are decellularized with 4000 ml degassed water containing 1% SDS for at least about 6 hours using a pump for recirculation. The degassed water was prepared by heating deionized water to about 65° C. under low vacuum for about an hour. The hearts are then washed with degassed deionized water for about 1 hour and washed with degassed PBS for about 12 hours. The hearts are washed three times with 500 ml degassed PBS for at least about 24 hours each time using a pump for recirculation. The heart can then be perfused with 35 ml DNase I (70 U/ml) for about 1 hour using manual recirculation, and washed three times with 500 ml degassed PBS for at least about 24 hours each time using a pump for recirculation.

Livers are washed in 2000 ml PBS, disinfected with PAA, and perfused with 2000 ml of degassed 0.9% saline. The livers are decellularized with a total of about 10 L degassed water containing about 0.6% SDS for at least about 6 hours using a pump for a series of single flow through and recirculation baths. The degassed water was prepared by pass deionized water through a vacuum filter designed to degas the solution. The livers are then washed with degassed deionized water for about 1 hour and washed with degassed PBS for about 12 hours. The livers are washed three times with 2000 ml degassed PBS for at least about 2 hours each time using a pump for recirculation.

Decellularization with the degassed buffers produces fewer microbubbles than decellularization with non-degassed buffers.

### Triton X Decellularization

Hearts are washed with 200 ml PBS containing 100 U/ml Penicillin, 0.1 mg/ml Streptomycin, and 0.25 μg/ml Amphotericin B for at least about 20 mins with no recirculation. Hearts are then decellularized with 500 ml degassed water containing 5% Triton X and 0.1% ammonium hydroxide for at least 6 hours using a pump for recirculation. Degassed buffers are prepared as described above for PEG decellularization. Hearts are then perfused with degassed deionized water for about 1 hour and then with degassed PBS for about 12 hours. Hearts are washed by perfusing with 500 ml degassed PBS 3 times for at least 24 hours each time using a pump for recirculation. Hearts are then perfused with 35 ml DNase I (70 U/ml) for about 1 hour using manual recirculation, and washed three times in 500 ml degassed PBS for about 24 hours each time.

For initial experiments, the decellularization apparatus can be set up within a laminar flow hood. Hearts are perfused at a coronary perfusion pressure of 60 cm H₂O. Although not required, the hearts described in the experiments above are mounted in a decellularization chamber and completely submerged and perfused with degassed PBS containing antibiotics for 72 hours in recirculation mode at a continuous flow of 5 ml/min to wash out as many cellular components and detergent as possible.

Successful decellularization can be defined by a lack of myofilaments and nuclei in histologic sections. Successful preservation of vascular structures can be assessed by perfusion with 2% Evans Blue prior to embedding tissue sections. Highly efficient decellularization can take place by first perfusing a heart antegrade with an ionic detergent (1% sodium-dodecyl-sulfate (SDS), approximately 0.03 M) dissolved in deionized H₂O at a constant coronary perfusion pressure and then can be perfused antegrade with a non-ionic detergent (l % Triton X-100) to remove the SDS and presumably to renature the extracellular matrix (ECM) proteins. Intermittently, the heart can be perfused retrogradely with phosphate buffered solution to clear obstructed capillaries and small vessels.

### Example 2: Evaluation of Decellularized Isolated Organs

To demonstrate intact vascular structures following decellularization, an at least partially decellularized heart can be stained via Langendorff perfusion with Evans Blue to stain vascular basement membrane and quantify macro- and micro-vascular density. Further, polystyrene particles can be perfused into and through a heart to quantify coronary volume, the level of vessel leakage, and to assess the distribution of perfusion by analyzing coronary effluent and tissue sections. A combination of three criteria are assessed and compared to isolated non-decellularized heart: 1) an even distribution of polystyrene particles, 2) significant change in leakiness at some level 3) microvascular density.

Fiber orientation can be assessed by the polarized-light microscopy technique of Tower et al. (2002, Fiber alignment imaging during mechanical testing of soft tissues, Ann Biomed Eng., 30(10):1221-33), which can be applied in real-time to a sample subjected to uniaxial or biaxial stress. During Langendorff perfusion, basic mechanical properties of an at least partially decellularized ECM are recorded (compliance, elasticity, burst pressure) and compared to freshly isolated hearts.

Histological Evaluation

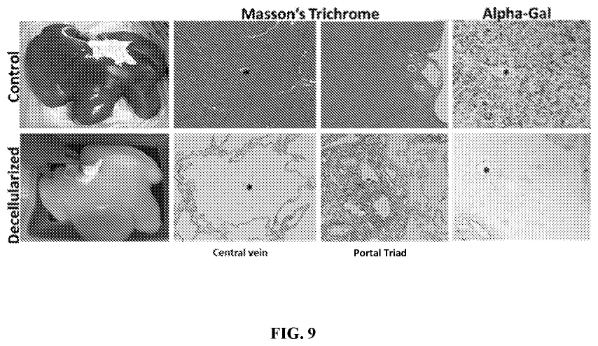

To evaluate the cellular content and histoarchitecture, representative samples of both native and decellularized kidneys, hearts and livers are processed for hematoxylin and eosin (H&E). The samples are fixed in 10% neutral buffered formalin solution at 4 C for 24 h. Subsequently, these are washed in distilled water, dehydrated in graded alcohol, embedded in paraffin and sectioned (5 mm) for staining. Tissue slides were stained with H&E following standard protocols. To exclude the presence of nuclear materials, the native kidneys and decellularized kidney scaffolds are processed for DNA-binding fluorescent staining by DAPI (40, 6-diamidino-2-Phenylindole (DAPI, Vector Laboratories, Burlingame, Calif.)) staining. Examination of stained sections was done under the immunofluorescent microscope (Olympus, Japan), FIG. 9.

Scanning Electron Microscopy (SEM)

For assessment of ultrastructure after the decellularization step, specimens of native kidneys and decellularized kidneys are processed for scanning electron microscopy. The specimens are washed in distilled water and fixed in cold 2.5% glutaraldehyde for 2 h at 48 C. Following washing with PBS to remove the residual glutaraldehyde, the samples are dehydrated by increasing concentrations of ethanol (30% ethanol, 50% ethanol, 70% ethanol, 90% ethanol, and 100% ethanol) for 20-30 min at room temperature. The samples are critical point dried using CO₂ and finally mounted on aluminum stubs using sticky carbon taps for imaging using scanning electron microscope (Carl Zeiss, Oberkochen, Germany).

Vascular Network Imaging

To evaluate the vascular network and kidney capsule integrity, radio-opaque contrast solution (Omnipaque 350, GE Healthcare, Piscataway, N.J.) can be perfused through the renal artery into the native and decellularized kidneys, contrast-radiography (KMC-950, Komed, Korea) can be employed 10 min after contrast reagent perfusion.

Quantification of Extracellular Matrix (ECM) Components

To check the effect of the decellularization process on ECM components, samples of native and decellularized renal tissues are digested in PBS containing 50 mg mL Proteinase K at 56° C. overnight. Then, the lysates are heat-inactivated at 90° C. for 10 min and centrifuged at 13,000×g for 10 min. Supernatants are collected and assayed for protein concentration using the BCA Protein Assay (Thermo Scientific, Rock-ford, IL). GAGs, collagen, and elastin levels are quantified in the lysate using the Blyscan sGAGs Assay Kit (Biocolor Ltd, Carrickfergus, UK), the Sircol Soluble Collagen Assay Kit (Bio-color Ltd), and Fastin Elastin Assay Kit (Biocolor Ltd), respectively according to the manufacturer's instructions.

Mechanical Testing

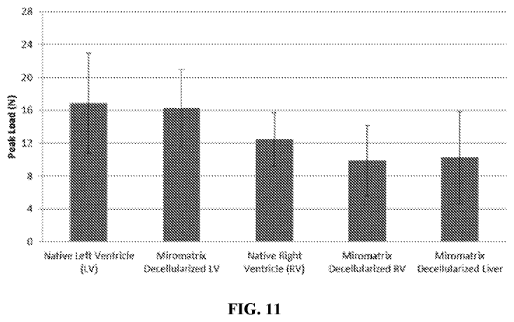

Crosses of myocardial tissue can be cut from the left ventricle of rats so that the center area can comprise approximately 5 mm×5 mm and the axes of the cross are aligned in the circumferential and longitudinal directions of the heart. The initial thickness of the tissue crosses are measured by a micrometer. Crosses are also cut from decellularized rat left ventricular tissue in the same orientation and with the same center area size. In addition, the mechanical properties of fibrin gels are tested, another tissue engineering scaffold used in engineering vascular and cardiac tissue. Fibrin gels are cast into cross-shaped molds with a final concentration of 6.6 mg of fibrin/ml. All samples are attached to a biaxial mechanical testing machine (Instron Corporation, Norwood, Mass.) via clamps, submerged in PBS, and stretched equibiaxially to 40% strain. In order to probe the static passive mechanical properties accurately, the samples are stretched in increments of 4% strain and allowed to relax at each strain value for at least 60 seconds. FIG. 11 depicts a plot of the peak load for the samples. Forces are converted to engineering stress by normalizing the force values with the cross sectional area in the specific axis direction (5 mmx initial thickness). Engineering stress can be calculated as the displacement normalized by the initial length. In order to compare the data between the two axes as well as between sample groups, a tangential modulus can be calculated as follows: [T(ε=40% strain)−T(ε=36% strain)]/4% strain, where T can be engineering stress and E can be engineering strain. The values for the tangential modulus are averaged and compared between the two axes (circumferential and longitudinal) as well as between groups.

### Example 3: Perfusion Recellularization of Isolated Solid Organs Using Degassed Media

### Human Umbilical Vein Endothelial Cells (HUVECS)

Perfusion recellularization of isolated solid organs can be performed by perfusing cells into an at least partially decellularized porcine heart by coronary perfusion. An at least partially decellularized porcine heart can be transferred to an isolated organ chamber and continuously perfused with oxygenized cell culture media under cell culture conditions (5% CO₂, 60% humidity, 37° C.). 120×10⁶ PKH labelled HUVECs (suspended in 50 ml of degassed endothelial cell growth media) are infused at 40 cm H₂O coronary perfusion pressure. Degassed endothelial cell growth media can be perfused by storing endothelial cell growth media as described in Example 1. Coronary effluent can be saved, and cells can be counted. The effluent can then be recirculated and perfused again to deliver a maximum number of cells. Recirculation can be repeated two times. After the third passage, approximately 90×10⁶ cells are retained within the heart. The heart can be continuously perfused with 500 ml of recirculating oxygenized endothelial cell culture media for 120 hours. The heart can then be removed and embedded for cryosectioning.

### Regenerative Cells

Perfusion recellularization of isolated solid organs can be performed by perfusing regenerative cells (embryonic stem cells, umbilical cord cells, adult-derived stem or progenitor cells, bone marrow-derived cells, blood-derived cells, induced pluripotent stem cells (iPSCs), mesenchymal stem cells (MSC), skeletal muscle-derived cells, multipotent adult progenitor cells (MAPC), cardiac stem cells (CSC), multipotent adult cardiac-derived stem cells) into an at least partially decellularized porcine heart by coronary perfusion. An at least partially decellularized porcine heart can be transferred to an isolated organ chamber and continuously perfused with oxygenized cell culture media under cell culture conditions (5% CO₂, 60% humidity, 37° C.). 120×10⁶ regenerative cells (suspended in 50 ml of cellular growth media) are infused at 40 cm H₂O coronary perfusion pressure. Coronary effluent can be saved, and regenerative cells are counted. The effluent can then be recirculated and perfused again to deliver a maximum number of cells. Recirculation can be repeated two times. After the third passage, approximately 90×10⁶ cells are retained within the heart. The heart can be continuously perfused with 500 ml of recirculating oxygenized endothelial cell culture media for 120 hours. The heart can then be removed and embedded for cryosectioning.

### Example 4: Reducing Particulate Formation

The presentation of a non-soluble white particulate can be noticed during the perfusion decellularization of isolated whole organs or portions thereof with some detergents such as SDS. Particulate forms and then can become trapped in the isolated organ or portion thereof during the decellularization. Reducing particulate formation can comprise significantly decreasing the amount of trace detergents or detergent remaining after decellularization. Reducing particulate formation can increase the ability to recellularize the isolated organ. Reducing particulate formation can decrease cytotoxicity of the decellularized matrix to introduced cells. Particulate can be formed by an insoluble interaction between native proteins and detergents. Reducing particulate can be performed by using solutions, controlling a mammal's eating habits, or their combination.

### Saline Solution

During isolated organ decellularization, isolated organs were immediately treated with detergent. The addition of saline was found to inhibit or prevent particulate formation. After excision from a mammal, isolated organs were flushed with 0.9% saline or PBS and then disinfected with peracetic acid. The isolated organs were primed with 0.9% saline prior to decellularization with detergent and placed into a 0.9% saline or PBS bath prior to detergent perfusion.

In particular, the porcine liver was washed in 0.9% saline or PBS followed by a peracetic acid wash. The washed liver was then submerged in a 0.9% saline or PBS bath. For decellularization, the liver was decellularized with 5000 ml water containing 1% SDS for at least about 6 hours using a pump for recirculation. The liver was then washed with deionized water for about 1 hour and washed with PBS for about 12 hours. The livers were washed three times with 500 ml PBS for at least about 24 hours each time using a pump for recirculation. The liver was then perfused with 35 ml DNase I (70 U/ml) for about 1 hour using manual recirculation, and washed three times with 500 ml PBS for at least about 24 hours each time using a pump for recirculation.

Non-Fasted Mammal

Particulate formation can be reduced in a mammal by ensuring that prior to euthanasia that the mammal has been fed in the last 8 hours.

4 hours after feeding, a pig was euthanized, and its liver was acquired. The porcine liver was washed in 200 ml PBS containing 100 U/ml Penicillin, 0.1 mg/ml Streptomycin, and 0.25 μg/ml Amphotericin B for at least about 20 mins with no recirculation. The liver was decellularized with 500 ml water containing 1% SDS for at least about 6 hours using a pump for recirculation. The liver was then washed with deionized water for about 1 hour and washed with PBS for about 12 hours. The liver was washed three times with 500 ml PBS for at least about 24 hours each time using a pump for recirculation. The liver was then washed three times with 500 ml PBS for at least about 24 hours each time using a pump for recirculation. It was observed that a high percentage of decellularized livers contained slight to heavy particulate where pigs were fasted or had limited food access. Non-fasted or isolation from recently fed animals significantly dropped particulate formation from 79% to 4.6% per decellularization lot, Table 2. % particulate refers to the amount of total grafts run that contained particulate upon visual inspection. For the DL-071116 lot, 11 of 30 grafts run had particulate noted, corresponding to 37% of the lot.

TABLE 2
Percent Particulate in decellularized porcine livers.

| | Particulate | | |
|---|---|---|---|
| | (Livers with | | |
| | particulate: | | |
| | livers with <5% | | |
| Decellularization | particulate: | % | |
| Lot | Total livers) | particulate | Fasted? |
| 1 | 5:06:30 | 37% | Yes |
| 2 | 5:14:24 | 79% | Yes |
| 3 | 5:12:25 | 68% | Yes |
| 4 | 5:11:24 | 67% | Yes |
| 5 | 9:14:26 | 96% | Yes |
| 6 | 7:18:26 | 96% | Yes |
| 7 | 15:08:30 | 77% | Yes |
| 8 | 7:00:07 | 100% | Yes |
| 9 | 0:02:27 | 7% | No |
| 10 | 0:01:30 | 3% | No/Not |
| | | | noted |
| 11 | 1:02:25 | 12% | No |
| 12 | 0:02:30 | 7% | No |
| 13 | 0:00:27 | 0% | No |
| 14 | 0:00:29 | 0% | No |
| 15 | 0:01:29 | 3% | No |

Whole livers were isolated from fasted or non-fasted animal, perfusion decellularized with SDS and the level of particulate formation was visually quantified. Livers isolated from non-fasted animals demonstrated very low levels of particulate formation compared to fasted animals.

Non-Fasted Mammal Plus Saline Bath

Livers collected from abattoirs under standard conditions which are normally fasted were washed in 0.9% saline or PBS followed by a peracetic acid disinfection. The washed livers were then submerged in a 0.9% saline or PBS bath, and transferred to a 0.9% saline solution bath until decellularization was initiated, with the liver remaining in the initial 0.9% saline bath. For decellularization, the liver was decellularized with at least 1000 ml water containing 0.6% SDS for at least about 2 hours using a pump for initial single flow though the liver and then transferring to a new bath containing at least 3000 ml water containing 0.6% SDS with recirculation for at least about 6 hours. The liver was then washed with deionized water for about 1 hour and washed with PBS for about 12 hours. The livers were washed three times with 500 ml PBS for at least about 24 hours each time using a pump for recirculation. The liver was then perfused with 35 ml DNase I (70 U/ml) for about 1 hour using manual recirculation, and washed three times with 500 ml PBS for at least about 24 hours each time using a pump for recirculation. Particulate formation was significantly reduced when harvested livers were placed into a 0.9% saline bath at the initiating of decellularization with a water solution containing 0.6% SDS (Table 3).

TABLE 3
Percent Particulate formation in Saline Bath

| | | Particulate | Total |
|---|---|---|---|
| Lot | Saline Bath | Formation | Livers |
| 16 | No | 40% | 10 |
| 17 | No | 50% | 10 |
| 18 | Yes | 3% | 30 |
| 19 | Yes | 0% | 24 |
| 20 | Yes | 0% | 28 |
| 21 | Yes | 0% | 35 |
| 22 | Yes | 0% | 13 |
| 23 | Yes | 0% | 20 |

Particulate formation was visually detected in less than 3% of livers when livers were in a 0.9% saline bath at the initiation of decellularization, compared to over 40% when a saline bath wasn't used.

Recellularization of Livers

HUVEC Cell Culture and Seeding of Decellularized Liver Constructs:

Human umbilical vein endothelial cells were cultured in antibiotic-free EGM-2 medium in tissue culture flasks at 37° C. and 5% CO₂ and passaged with 0.25% trypsin at 90-100% confluency according to manufacturer's protocol. The highest passage used for seeding liver grafts was passage 11. Decellularized porcine livers utilizing degassed decellularization were placed in a custom bioreactor containing 800 ml of media cell, connected to the perfusion inlet via the suprahepatic vena cava, and perfused at 12 mmHg with culture media prior to seeding. HUVECs were resuspended in 100 ml of media and seeded through the suprahepatic vena cava. The infused cell suspension was left under static conditions for one hour and then perfusion was restarted. After 24 hours, perfusion was changed from the suprahepatic vena cava to the portal vein and the seeding protocol was repeated. Re-endothelialized grafts were maintained in a continuous perfusion loop with metabolites (glucose, lactate, glutamine, glutamate and ammonia) monitored daily in collected media samples using a BioProfile FLEX analyzer. Culture media was exchanged and the volume increased depending on the rate of glucose depletion in the circulating medium to ensure 24 hour glucose levels above 500 mg/L.

Histological Analysis:

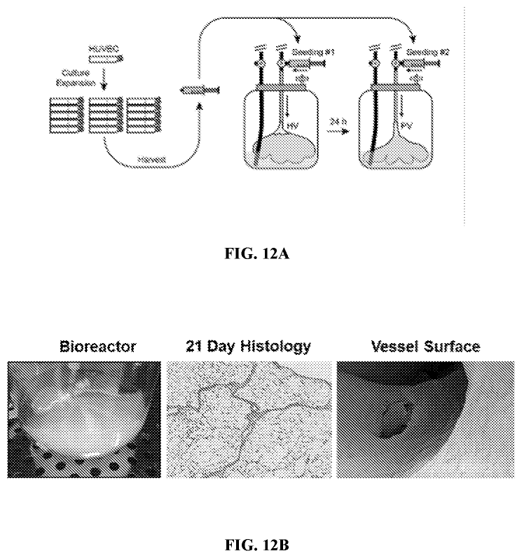

Tissue samples analyzed in this study were perfused with PBS and fixed with 10% Neutral Buffered Formalin (VWR 16004-128). Fixed tissues were paraffin embedded, sectioned and stained using standard histologic techniques. FIG. 12A depicts a representation of endothelial seeding used in the histological analysis. FIG. 12B depicts representative images of the bioreactor, histology after 21 days, and an exemplary vessel surface.

Recellularization of Kidneys with Whole Liver Isolation

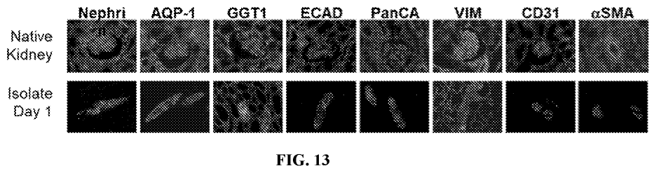

Decellularized porcine kidneys utilizing degassed decellularization were placed in a custom bioreactor containing 800 ml of media cell, connected to the perfusion inlet via the renal artery, and perfused at 12 mmHg with culture media prior to seeding. While kidney isolates were resuspended in 100 ml of media and seeded through the ureter. The infused cell suspension was left under static conditions for one hour and then perfusion was restarted. Recellularized kidneys were maintained in a continuous perfusion loop with metabolites (glucose, lactate, glutamine, glutamate and ammonia) monitored daily in collected media samples using a BioProfile FLEX analyzer. Culture media was exchanged and the volume increased depending on the rate of glucose depletion in the circulating medium to ensure 24 hour glucose levels above 500 mg/L. As depicted in FIG. 13, immunofluorescent staining demonstrates the distribution and engraftment of native kidney cells throughout the kidney in the proper location.

### Example 5: Isolated Organ Decellularization

Isolated organs, such as liver, spleen, lung, kidney, and heart are flushed with a hypertonic solution (e.g., 5× saline). The isolated organs can then be disinfected. For instance, isolated organs can be placed in a disinfection bath containing either a peracid alone, or a solution of a peracid and saline. Specifically, the disinfection bath can contain saline, a peracid (e.g., peracetic acid), and NaOH and can have a pH between 6-7. A disinfection bath can contain a disinfection solution that can be made, by for example, adding 2 liters of 10× saline (9% NaCl) to 18 liters water to create 1× saline (0.9% NaCl) and adding 80 mL of 15% peracetic acid and 51 mL of 5N NaOH to make a final concentration of 600 ppm PAA supplemented in 0.9% NaCl at pH of 6.12. Isolated organs can be placed in the disinfection bath for various times (e.g., for 30 minutes, 1 hour, 2 hours, 3 hours, 4 hours, 6 hours, 10 hours, 12 hours, 15 hours, 18 hours, 24 hours, overnight, for at least a day, for two days, for three days, or longer) with different concentrations of a peracid (e.g., 50 parts per million (ppm), 100 ppm, 150 ppm, 200 ppm, 250 ppm, 300 ppm, 350 ppm, 400 ppm, 450 ppm, 500 ppm, 550 ppm, 600 ppm, 650 ppm, 700 ppm, 750 ppm, 800 ppm, 850 ppm, 900 ppm, 1000 ppm, 1200 ppm, 1400 ppm, 1500 ppm, 1700 ppm, 1800 ppm, 1900 ppm, 2000 ppm, or greater than 2000 ppm). After the isolated organs are removed from the disinfection bath, the isolated organs are then decellularized. The isolated organs can be decellularized with a solution containing a detergent (e.g., sodium dodecyl sulfate (SDS)) or a solution containing a detergent and a peracid. SDS in the decellularization solution can range from, for example, about 0.2%-2%. In some embodiments, the decellularization can be performed with 0.6% SDS or 0.9% SDS. The peracid present in the solution during decellularization can range from about 25 ppm-3000 ppm. In some embodiments, the peracid can be present at 50 ppm, 100 ppm, 200 ppm, 400 ppm, 500 ppm, 600 ppm, 650 ppm, 700 ppm, 1000 ppm, 1500 ppm, or 2000 ppm. In some embodiments, the isolated organ can be perfused with a solution containing PAA and SDS.

In short, a perfusable liver was flushed with 5× saline and then placed in a disinfection bath containing 600 ppm peracetic acid (PAA). Peracetic acid (PAA) was added to 1× saline solution (0.9% NaCl) to make a final solution containing 600 ppm PAA at pH of 6.12. After the liver was removed from the disinfection bath, the liver was perfused with a solution containing PAA and SDS for a period of time (e.g., for about 1 hour, 2 hours, 3 hours, 4 hours, 5 hours, 6 hours, 7 hours, 8 hours, 9 hours, 10 hours, 12 hours, 15 hours, 16 hours, 20 hours, 24 hours, 30 hours, 35 hours, 40 hours, 45 hours, 46 hours, 47 hours, 48 hours, 49 hours, 50 hours, or longer). The PAA in the solution used for perfusion decellularization of the liver ranged from about 50 ppm-3000 ppm.

### Example 6: Complete Decellularization of an Isolated Organ with a Radical Generating Compound

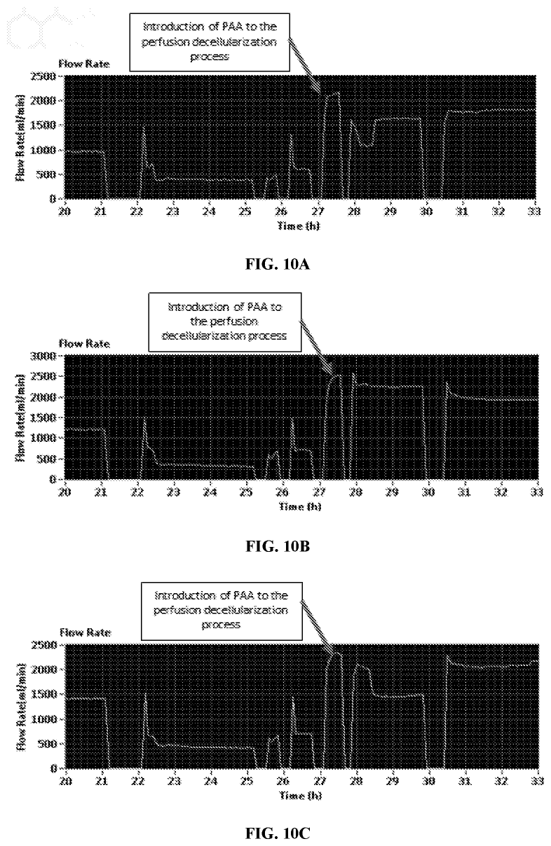

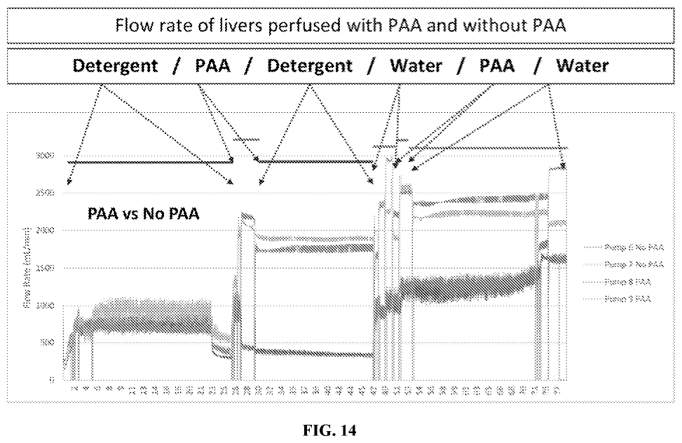

The use of a radical generating compound in a solution during decellularization of an isolated organ affected the flow rate and the decellularization process of the isolated organ without compromising the integrity matrix (FIGS. 10A-C). A liver from a pig was obtained and cannulated. The native vasculature of a porcine liver was cannulated and perfused with a mild detergent solution in order to decellularize the liver. The detergent solution further contained a radical generating compound (e.g., peracetic acid). It was observed that after the detergent solution containing a radical generating compound was introduced to the isolated organ at a set pressure (e.g., 12 mmHg), the flow rate increased (e.g., increased to at least 500 m/min, 1000 mL/min, 1500 mL/min, 2000 mL/min, 2500 mL/min, or greater) and the decellularization process decreased (FIGS. 10A-C). This experiment showed that the use of a radical generating compound (e.g., peracetic acid) at a concentration of about 50-2000 ppm resulted in an increase in the flow rate during perfusion decellularization at a predetermined pressure as compared to an otherwise comparable decellularization process without using a radical generating compound. FIG. 14 illustrates the improvement in flowrate by incorporation of a radical generating compound into a decellularization workflow. As shown in FIG. 14, the overall flowrate of each step in the decellularization workflow was improved after the addition of the radical generating compound. This improvement in workflow was propagated throughout the decellularization workflow, even to perfusion steps that did not include radical generating compound. Indeed, this increase in the flow rate at a set pressure occurred during the perfusion with a radical generating compound (e.g., PAA) and was retained in subsequent perfusion steps, even in steps that did not include a radical generating compound. This increase in flow rate resulted in a more complete decellularization of the isolated organ without affecting the integrity matrix.

Overall, the results showed that more cells were removed in a shorter period of time and that the increase in flow rate resulted in a purer and more complete decellularization of the isolated organ. Isolated organs are dense with vascular capillaries with most cells located in close proximity to a capillary. Thus, perfusion of a cannulated isolated organ with a solution containing a radical generating compound and the subsequent increase in the flow rate resulted in an increased effective surface area of the detergent and a decreased time required to expel the native cellular material through the venous system. The results also showed that the increase in the flow rate or the addition of a radical generating compound during decellularization did not significantly affect or compromise the integrity matrix.

### Example 7: Radical Generating Compounds Improve Isolated Organ Decellularization

An isolated mammalian liver was placed in a reservoir bin containing SDS for about 1-24 hours. The liver was then placed in a reservoir bin containing hepatic SDS for about 1-10 hours and then placed in a bin containing water (H₂O). The liver was removed from the water bin and placed in a bin containing a radical generating compound (PAA) followed by a bin containing a solution of PAA in SDS. The PAA ranged from about 0.1% to about 30%. The PAA in SDS ranged from about 25 ppm-3000 ppm. The liver was then placed in SDS followed by additional steps including washing with hepatic H₂O, H₂O, H₂O and PBS, further treatment with PAA, and further washing. The duration of SDS perfusion ranged from about 10-100 hours (e.g., 25-60 hours) and the duration of PAA perfusion ranged from about 1-20 hours (e.g., 2-10 hours).

### Example 8: Quantitation of Radical Generating Compounds Improvement of Decellularization

An isolated mammalian liver was decellularized in the presence or absence of the radical generating compound PAA as described in Example 7. In order to quantitate the improvement in the decellularization process after the addition of the PAA, the residual concentration of DNA was determined from the decellularized lobe tips. Approximately 10-30 mg of tissue was collected and digested with Proteinase K in buffer at 60° C. for 8-30 hours. Chloroform was added at a 2:5 ratio with respect to tissue, and the mixture was centrifuged for 10 minutes at 14,000×g. The supernatant was collected and a DNA stripping solution was added to the supernatant. The suspension was incubated at 60° C. for 5-10 minutes. Precipitation solution was added and the supernatant was collected after centrifuging at 14,000×g for 5 minutes. The DNA was precipitated using 100% isopropanol and Mussel Glycogen followed by centrifugation at 14,000×g for 5 minutes. The pellet was washed with 70% ethanol and dissolved in a tris buffer. The amount of DNA in each sample was quantified using a Qubit™ dsDNA BR Assay Kit and Qubit® 2.0 Fluorimeter.

TABLE 4

| | | Sample | Measured | Total | DNA |
|---|---|---|---|---|---|
| | Lobe/ | Weight | DNA Conc. | DNA | (ng/mg |
| DNA Source | Location | (mg) | (μg/mL) | (ng) | tissue) |
| Pump 6-No PAA | Lobe Tips | 64 | 0.872 | 1308.0 | 204.4 |
| Pump 7-No PAA | Lobe Tips | 79 | 1.26 | 1890.0 | 239.2 |
| Pump 8-No PAA | Lobe Tips | 84 | 0.0813 | 122.0 | 14.5 |
| Pump 9-No PAA | Lobe Tips | 73 | 0.103 | 154.5 | 21.2 |

Table 4 above depicts the residual DNA concentration after decellularization. Evaluating the residual DNA content, an indication regarding the quality and/or completeness of decellularization, demonstrated a 10-fold reduction in residual DNA with the addition of PAA. Accordingly, this demonstrates that addition of a radical generating compound improved the completeness of a decellularization process.

While exemplary embodiments have been shown and described herein, it will be obvious to those skilled in the art that such embodiments are provided by way of example only. Numerous variations, changes, and substitutions will occur to those skilled in the art. It should be understood that various alternatives to the embodiments described herein can be employed. It is intended that the following claims define the scope of the disclosure and that methods and structures within the scope of these claims and their equivalents be covered thereby.

What is claimed is:

1. A method comprising contacting an isolated organ or portion thereof with a liquid, wherein:
(a) the liquid is at least partially degassed;
(b) the liquid comprises a detergent and a radical-generating compound;
(c) the isolated organ or portion thereof is from a mammal that was fed within about ten hours prior to removal of the isolated organ or portion thereof; or
(d) any combination of (a)-(c).

2. The method of claim 1, comprising at least two of: (a), (b) or (c).

3. The method of claim 1 or claim 2, comprising (a), (b), and (c).

4. The method of any one of claims 1-3, wherein the isolated organ or portion thereof is from a mammal.

5. The method of claim 4, wherein the mammal is a non-human mammal.

6. The method of claim 5, wherein the non-human mammal is a pig, a sheep, a goat, a cow, a dog, a cat, or a monkey.

7. The method of claim 4, wherein the mammal is a human mammal.

8. The method of any one of claims 1-7, wherein the isolated organ or portion thereof is at least part of a liver, a lung, a heart, a kidney a bladder, a pancreas, a spleen, a uterus, a portion of any of these, or any combination thereof.

9. The method of any one of claims 1-8, wherein the contacting comprises (i) perfusing the liquid in at least a portion of the isolated organ or portion thereof, (ii) injecting the liquid in at least a portion of the isolated organ or portion thereof, (iii) spraying the liquid on at least a portion of the isolated organ or portion thereof, (iv) submerging the isolated organ or portion thereof in the liquid, or (v) any combination thereof.

10. The method of any one of claims 1-9, wherein prior to, during, or after the contacting, the isolated organ or portion thereof is cannulated.

11. The method of any one of claims 1-10, comprising (a), wherein the liquid comprises an at least partially degassed decellularization media, thereby forming an at least partially decellularized isolated organ or portion thereof.

12. The method of claim 11, wherein the at least partially decellularized isolated organ or portion thereof comprises an extracellular matrix.

13. The method of claim 12, wherein the extracellular matrix comprises a vasculature bed.

14. The method of claim 13, wherein following the contacting, the vasculature bed remains at least partially intact.

15. The method of any one of claims 12-14, wherein the extracellular matrix has an increased compressive modulus relative to an otherwise comparable extracellular matrix produced by contact with an otherwise comparable non-degassed liquid for a comparable time period.

16. The method of any one of claims 1-15, comprising (a).

17. The method of claim 16, wherein the isolated organ or portion thereof is contacted by the at least partially degassed liquid for at least about two hours.

18. The method of claim 16, wherein a dissolved gas in the at least partially degassed liquid has a concentration of less than about 1 milligram (mg) per liter (L).

19. The method of claim 18, wherein the dissolved gas is selected from the group consisting of: oxygen, nitrogen, carbon monoxide, carbon dioxide, a noble gas, and any combination thereof.

20. The method of claim 16, wherein the isolated organ or portion thereof after the contacting comprises (a) fewer air emboli, (b) fewer microbubbles, (c) less pigmentation, or (d) any combination thereof relative to an otherwise comparable isolated organ or portion thereof contacted for a comparable amount of time with an otherwise comparable liquid that has not been at least partially degassed.

21. The method of claim 16, wherein the at least partially degassed liquid comprises an at least partially degassed decellularization media, thereby forming an at least partially decellularized isolated organ or portion thereof.

22. The method of claim 21, wherein the at least partially decellularized isolated organ or portion thereof after the contacting with the at least partially degassed decellularization media contains fewer cells than an otherwise comparable at least partially decellularized isolated organ or portion thereof contacted with an otherwise comparable non-degassed decellularization media for a comparable period of time.

23. The method of claim 21, wherein an amount of time sufficient to produce the at least partially decellularized isolated organ or portion thereof with the at least partially degassed decellularization media is less than an amount of time to produce an otherwise comparable at least partially decellularized isolated organ or portion thereof with an otherwise comparable non-degassed decellularization media.

24. The method of any one of claims 1-15, comprising (b).

25. The method of claim 24, wherein the radical generating compound is of formula R¹—O—O—R², wherein R¹ and R² are independently H, C₁-C₆ alkyl, substituted C₁-C₆ alkyl, aryl, substituted aryl, benzyl, substituted benzyl, C(═O)A¹, C(═O)OA², wherein A¹ and A² are independently H, C₁-C₆ alkyl or substituted C₁-C₆ alkyl.

26. The method of claim 25, wherein R¹ is H and R² is C(═O)CH₃.

27. The method of claim 24, wherein the radical generating compound is a peracid.

28. The method of claim 27, wherein the peracid comprises: peroxyacetic acid, peroxyoctanoic acid, a sulfoperoxycarboxylic acid, peroxysulfonated oleic acid, peroxyformic acid, peroxyoxalic acid, peroxypropanoic acid, peroxybutanoic acid, peroxypentanoic acid, peroxyhexanoic acid, peroxyadipic acid, peracetic acid, perlactic acid, peroxycitric, peroxybenzoic acid, or any combination thereof.

29. The method of claim 26, wherein the peracid comprises the peracetic acid.

30. The method of any one of claims 24-29, wherein the peracid is present in the liquid in an amount that comprises at least about 10 ppm, 25 ppm, 50 ppm, 75 ppm, 90 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 900 ppm, 1000 ppm, 1200 ppm, 1400 ppm, 1500 ppm, 1700 ppm, 2000 ppm, 2200 ppm, 2500 ppm, 2750 ppm, 3000 ppm, 3200 ppm, 3500 ppm, 3750 ppm, or 4000 ppm.

31. The method of any one of claims 24-30, wherein the contacting produces an at least partially decellularized isolated organ or portion thereof.

32. The method of claim 31, wherein the at least partially decellularized isolated organ or portion thereof comprises a reduced amount of cells as compared to an amount of cells in an otherwise comparable at least partially decellularized isolated organ or portion thereof contacted with an otherwise comparable liquid that does not comprise a peracid for a comparable time period.

33. The method of claim 32, wherein the reduced amount of cells comprises a reduction of at least about 85%, at least about 90%, at least about 92%, at least about 95%, at least about 97%, at least about 98%, at least about 99%, or at least about 99.5% of cells, relative to the amount of cells in the otherwise comparable at least partially decellularized isolated organ or portion thereof.

34. The method of any one of claims 24-33, wherein the contacting comprises perfusing, and wherein a flow rate of perfusing is increased compared to a flow rate of perfusing of an otherwise comparable liquid that does not comprise the peracid.

35. The method of claim 34, wherein the flow rate is increased without substantially compromising an integrity of extracellular matrix of the isolated organ or portion thereof.

36. The method of any one of claims 1-15, comprising (c), wherein the liquid comprises a decellularization media, thereby at least partially decellularizing the isolated organ or portion thereof.

37. The method of claim 36, wherein the decellularizing proceeds with a reduced particulate level in the isolated organ or portion thereof, as compared to a particulate level produced in an otherwise comparable decellularizing of an otherwise comparable isolated organ or portion thereof removed from an animal which had not been fed within about 10 hours prior to removal of the isolated organ or portion thereof from the animal.

38. The method of claim 37, wherein the particulate level is reduced by at least about: 90%, 85%, 80%, 75%, 70%, 65%, 60%, 55%, 50%, 45%, 40%, 35%, 30%, 25%, 20%, 15%, 10%, 5%, 10% as compared to the particulate level produced in the otherwise comparable decellularizing.

39. The method of claim 37 or 38, wherein the reduced particulate level is determined by visual examination, determining an outflow, determining turbidity, microscopy analysis, or any combination thereof.

40. The method of any one of claims 1-39, further comprising washing the isolated organ or portion thereof with a wash media prior to the contacting.

41. The method of claim 40, wherein the wash media comprises a hypertonic solution.

42. The method of claim 41, wherein the hypertonic solution comprises saline.

43. The method of claim 40, wherein the wash media comprises a disinfecting solution.

44. The method of any one of claims 1-43, further comprising contacting the isolated organ or portion thereof with a second liquid.

45. The method of claim 44, wherein the contacting with the second liquid occurs after the contacting with the liquid.

46. The method of claim 44 or 45, wherein the second liquid is an at least partially degassed liquid.

47. The method of any one of claims 44-46, wherein the second liquid comprises a wash media.

48. The method of any one of claims 44-47, wherein the isolated organ or portion thereof contains less than about 1% weight per weight of a detergent after contacting of the isolated organ or portion thereof with the second liquid.

49. The method of claim 46, wherein after the contacting with the second liquid, blood flows through a greater volume of a vasculature of the at least partially decellularized isolated organ or portion thereof as compared to a volume of vascular of an otherwise comparable at least partially decellularized isolated organ or portion thereof produced using an otherwise comparable non-degassed second liquid.

50. The method of any one of claims 11-49, further comprising contacting the at least partially decellularized isolated organ or portion thereof with a regeneration media.

51. The method of claim 50, wherein the regeneration media is an at least partially degassed cellular regeneration media.

52. The method of claim 51, wherein the contacting with the at least partially degassed cellular regeneration media forms an at least partially recellularized organ or portion thereof.

53. The method of claim 51, wherein the at least partially degassed cellular regeneration media comprises a population of regenerative cells.

54. The method of claim 51, wherein the contacting with the at least partially degassed cellular regeneration media results in a higher degree of recellularization relative to a degree of recellularization of an otherwise comparable at least partially decellularized organ or portion contacted with an otherwise comparable non-degassed cellular regeneration media.

55. An at least partially decellularized isolated organ or portion thereof generated by the method of any one of claims 1-49.

56. An at least partially recellularized mammalian isolated organ or portion thereof generated by the method of any one of claims 50-54.

57. A system comprising (a) the at least partially decellularized isolated organ or portion generated by the method of any one of claims 1-49, (b) a device for moving fluid, (c) a cannula, and (d) a port.

58. The system of claim 57, further comprising a degassed decellularization media.

59. The system of claim 57, further comprising a degassed wash media.

60. A kit comprising the at least partially decellularized isolated organ or portion thereof generated by the method of any one of claims 1-49 in a container.

61. The kit of claim 60, further comprising, (a) at least one cannula, (b) a liquid, (c) instructions for recellularizing the at least partially decellularized isolated organ or portion thereof, (d) instructions for introducing an at least partially recellularized isolated organ or portion thereof in a subject, or (e) any combination thereof.

62. The kit of claim 60 or 61, wherein the container is a packaging.
